(19) United States
(12) Patent Application Publication
(10) Pub. No.: US 2010/0179127 A1
(43) Pub. Date: Jul. 15, 2010

(54) PYRIMIDINE DERIVATIVES

(76) Inventors: Philipp Floersheim, Basel (CH); Wolfgang Froestl, Ecublens (CH); Sebastien Guery, Verona (IT); Kiemens Kaupmann, Basel (CH); Manuel Koller, Schliern (CH)

Correspondence Address: FOLEY AND LARDNER LLP;SUITE 500, 3000 K STREET NW, WASHINGTON, DC, 20007, US

(21) Appl. No.: 11/993,630

(22) PCT Filed: Jun. 23, 2006
(86) PCT No.: PCT/EP06/06083

§ 371 Date: Dec. 21, 2007

(30) Foreign Application Priority Data
Jun. 23, 2005 (GB) 0512844.2

(51) Int. Cl. A61K 31/505; C07D 239/56; A61K 31/513; C07D 239/47; C07D 239/553; C07D 239/30; C07D 239/42; C07D 239/48; C07D 405/10; A61K 31/506; C07D 401/04; C07D 487/04; A61K 31/519; C07D 413/10; C07D 403/12; A61K 31/55; A61P 25/24; A61P 25/18; A61P 25/30; A61P 25/00; A61P 25/08; A61P 25/22

(52) U.S. Cl. 514/212.08; 544/313; 514/274; 544/317; 544/319; 514/269; 544/322; 514/256; 544/334; 544/326; 544/328; 544/250; 514/267; 540/524

(57) ABSTRACT

The invention relates to novel heterocyclic compounds of the formula

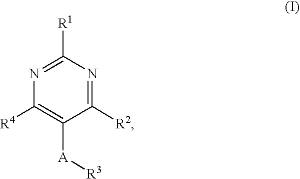

in free base form or in acid addition salt form, in which R¹, R², R³, R⁴ and A are as defined in the specification, to their preparation, to their use as medicaments and to medicaments comprising them.

The present invention relates to novel heterocyclic compounds, to their preparation, to their use as medicaments and to medicaments comprising them.

More particularly the invention relates to a compound of the formula

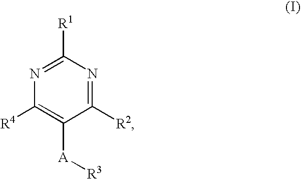

in free base form or in acid addition salt form, wherein
R¹ represents alkyl, halogenalkyl, alkoxy, halogenalkoxy, alkylthio, halogenalkylthio, alkylamino or halogenalkylamino;
R² represents halogen, hydroxy or substituted amino, the substituent(s) being selected from the group consisting of hydrogen, unsubstituted or substituted alkyl, unsubstituted or substituted cycloalkyl, unsubstituted or substituted bicycloalkyl, unsubstituted or substituted adamantyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl;
R³ represents halogen, halogenalkyl, nitro, unsubstituted or substituted aryl or unsubstituted or substituted heteroaryl;
R⁴ represents hydrogen, halogen, hydroxy, alkynyl, trialkylsilylalkynyl or substituted amino, the substituent(s) being selected from the group consisting of hydrogen, unsubstituted or substituted alkyl, unsubstituted or substituted cycloalkyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl; and
A represents a bond, alkandiyl, alkendiyl or alkyndiyl; and
   wherein additionally the amino nitrogen atom of a substituted amino group R² can be connected via a direct bond or via a carbonyl group with a ring carbon atom of an unsubstituted or substituted aryl or an unsubstituted or substituted heteroaryl group R³.

Preferably the invention relates to a compound of the formula I, in free base form or in acid addition salt form, wherein
R¹ represents alkyl, halogenalkyl or alkylthio;
R² represents halogen, hydroxy or mono-substituted amino, the substituent being selected from the group consisting of unsubstituted cycloalkyl, unsubstituted bicycloalkyl, unsubstituted adamantyl and heterocyclyl mono-substituted by oxo;
R³ represents halogen, halogenalkyl, nitro, unsubstituted or substituted phenyl, unsubstituted or substituted pyridyl or unsubstituted or substituted pyrimidyl;
R⁴ represents hydrogen, halogen, hydroxy, alkynyl, trialkylsilylalkynyl or mono-substituted amino, the substituent being selected from the group consisting of unsubstituted or substituted cycloalkyl; and
A represents a bond, alkandiyl, alkendiyl or alkyndiyl; and
   wherein additionally the amino nitrogen atom of a mono-substituted amino group R² can be connected via a direct bond or via a carbonyl group with a ring carbon atom of an unsubstituted or substituted phenyl group R³.

In the present specification, the following definitions shall apply if no specific other definition is given:

“Alkyl” represents a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl.

“Cycloalkyl” represents a cyclic alkyl group, preferably represents a C₃₋₁₂cycloalkyl, particularly preferably represents a C₃₋₈cycloalkyl; for example, cyclopropyl, cyclobutyl, cyclopentyl, cyclohexyl, cycloheptyl, cyclooctyl, cyclododecanyl, with particular preference given to cyclopentyl, cyclohexyl and cycloheptyl. Cycloalkyl includes cycloalkyl-moieties, which are substitueted by one or more alkyl groups as defined above. Preferred is unsubstituted cycloalkyl.

“Alkandiyl” represents a straight-chain or branched-chain alkandiyl group bound by two different bonds to the molecule, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

“Alkendiyl” represents a straight-chain or branched-chain alkendiyl group bound by two different bonds to the molecule, it preferably represents a straight-chain or branched-chain C₂₋₆ alkendiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to CH═CH—, —CH═CH—CH₂—, —CH═CH—CH═CH—.

“Alkynyl” represents a straight-chain or branched-chain alkynyl group, preferably C₂₋₆alkynyl, for example, ethenyl, propargyl, 1-propynyl, isopropenyl, 1-(2- or 3) butynyl, 1-(2- or 3) pentenyl, 1-(2- or 3) hexenyl, etc., preferably represents C₂₋₄alkynyl and particularly preferably represents ethynyl.

“Alkyndiyl” represents a straight-chain or branched-chain alkyndiyl group bound by two different bonds to the molecule, it preferably represents —CC—.

“Aryl” represents an aromatic hydrocarbon group, preferably a C₆₋₁₀ aromatic hydrocarbon group; for example phenyl, naphthyl, especially phenyl. An aryl group may be substituted by one or more substituents selected from the group consisting of alkyl, halogenalkyl, alkoxy, halogenalkoxy, methylenedioxy (bound to adjacent ring carbon atoms), ═N—O—N═ (bound to adjacent ring carbon atoms), carboxy, alkoxycarbonyl, aminocarbonyl, halogen, nitro, cyano, alkylsulfonyl, amino, alkylcarbonylamino, —N═N—N(dialkyl), —P(═O)(dialkoxy) and —P(═O)(OH)OH.

“Aralkyl” denotes an “Aryl” bound to an “Alkyl” (both as defined above) an represents, for example benzyl, α-methylbenzyl, 2-phenylethyl, α,α-dimethylbenzyl, especially benzyl.

“Heteroaryl” represents aromatic ring system containing at least one hetero atom. Preferably, heteroaryls consist of 5 to 11 ring atoms of which 1-3 ring atoms are hetero atoms. Heteroaryls may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heteroaryl may be substituted by one or more substituents selected from the group consisting of hydroxyl, Oxo (═O), Halogen, Nitro, Cyano, Alkyl, Alkandiyl, Alkenediyl, Alkoxy, Alkoxyalkyl, Alkoxycarbonyl, Alkoxycarbonylalkyl, Halogenalkyl, Aryl, Aryloxy, Arylalkyl.

“Heterocyclyl” represents a saturated, or partly saturated ring system containing at least one hetero atom. Preferably, heterocycles consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Hydroxy, Oxo (═O), Halogen, Nitro, Cyano, Alkyl, Alkandiyl, Alkenediyl, Alkoxy, Alkoxyalkyl, Alkoxycarbonyl, Alkoxycarbonylalkyl, Halogenalkyl, Aryl, Aryloxy, Arylalkyl.

Examples of heterocyclyl or heteroaryl moieties are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, 4-piperidino-piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. benzimidazole, indole, isoindole, cumarine, cumaronecinoline, isochinoline, cinnoline and the like.

“Hetero atoms” are atoms other than Carbon and Hydrogen, preferably Nitrogen (N), Oxygen (O) or Sulfur (S).

“Halogen” represents Fluoro, Chloro, Bromo or Iodo, preferably represents Fluoro, Chloro or Bromo and particularly preferably represents Chloro.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”. The same considerations apply to other expressions like Aryloxy, cycloalkylcarbonyl, heterocyclylalkyl.

Compounds of formula (I) exist in free or acid addition salt form. In this specification, unless otherwise indicated, language such as “compounds of formula (I)” is to be understood as embracing the compounds in any form, for example free base or acid addition salt form. Salts which are unsuitable for pharmaceutical uses but which can be employed, for example, for the isolation or purification of free compounds of formula (I), such as picrates or perchlorates, are also included. For therapeutic use, only pharmaceutically acceptable salts or free compounds are employed (where applicable in the form of pharmaceutical preparations), and are therefore preferred.

On account of the asymmetrical carbon atom(s) that may be present in the compounds of formula (I) and their salts, the compounds may exist in optically active form or in form of mixtures of optical isomers, e.g. in form of racemic mixtures or diastereomeric mixtures. All optical isomers and their mixtures, including the racemic mixtures, are part of the present invention.

In preferred embodiments, the invention relates to a compound of the formula I, in which any variable has one of the meanings given in the Examples hereinafter, in free base form or in acid addition salt form, which preferred embodiments are for each variable preferred independently, collectively or in any combination or sub-combination.

In especially preferred embodiments, the invention relates to one or more than one of the compounds of the formula I mentioned in the Examples hereinafter, in free base form or in acid addition salt form.
R¹ preferably represents methyl, ethyl, methylthio or trifluoromethyl, especially methyl.
R² preferably represents cyclopentylamino.
R³ preferably represents phenyl substituted by iodo or preferably by trifluoromethyl, especially in 4-position.
R⁴ preferably represents cyclopentylamino, chloro or especially hydrogen.
A preferably represents a single bond, —CH₂—CH₂—, —CH═CH— or —CC—, particularly preferably a single bond.

The abovementioned general or preferred radical definitions apply both to the end products of the formula (I) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formula (I) which contain a combination of the meanings mentioned above as being preferred.

Particular preference according to the invention is given to compounds of the formula (I) which contain a combination of the meanings listed above as being particularly preferred.

Very particular preference according to the invention is given to the compounds of the formula (I) which contain a combination of the meanings listed above as being very particularly preferred.

In a preferred embodiment, the invention provides a compound of formula (I) wherein the substituents R² and R⁴ are identical.

In a further preferred embodiment, the invention provides a compound of formula (I-A)

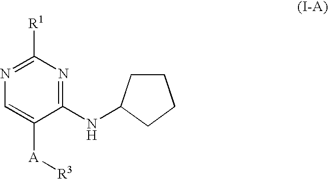

wherein R¹, R³ and A are as defined above.

In a further preferred embodiment, the invention provides a compound of formula (I-B)

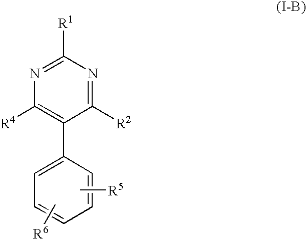

wherein
R¹, R² and R⁴ are as defined above and
R⁵ and R⁶ independently represent fluoro, chloro, bromo, jodo, cyano, nitro, amino, PO₃H₂, H₂NC(O), methyl, ethyl, n- or iso-propyl, n-, iso-, sec- or tert-butyl, fluormethyl, difluormethyl trifluormethyl, chlormethyl, dichlormethyl, methoxy, ethoxy, n- or iso-propoxy, n-, iso-, sec- or tert-butoxy, fluormethoxy, difluormethoxy, trifluormethoxy, chlormethoxy, dichlormethoxy, methoxycarbonyl, ethoxycarbonyl, trifluormethoxycarbonyl, C₁₋₄ methylthio, methylsulfinyl, methylsulfonyl, trifluormethylthio.

In a further aspect, the invention provides a process for the production of the compounds of formula I and their salts, which comprises

a: —in case A represents a single bond—the step of reacting a compound of formula (II)

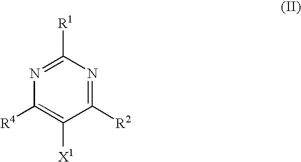

wherein R¹, R² and R⁴ are as defined above, and X¹ represents Br or I, with a compound of formula (III)

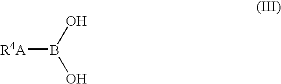

wherein R³ is as defined above and A represents a single bond, in a Suzuki type coupling reaction and recovering the resulting compound of formula (I) in free base or acid addition salt form; or
b: —in case A represents alkandiyl, alkendiyl or alkyndiyl—the step of reacting a compound of formula (II)

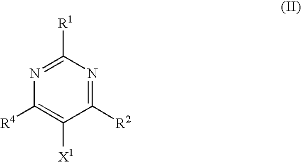

wherein R¹, R² and R⁴ are as defined above, and X¹ represents Br or I, with a compound of formula (IV)

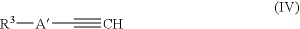

wherein R³ is as defined above and A′ represents a single bond (in case A represents C₂) or an alkandiyl which is two C atoms shorter than A in the compound of formula (IV), in a Sonogashira type coupling reaction, possibly followed by hydrogenation of the triple bond, and recovering the resulting compound of formula (I) in free base or acid addition salt form.

The Suzuki coupling according to process a) can be effected according to conventional methods. Typically, Palladium catalysts such as Pd(OAc)₂ in the presence of a bisphosphineligand or Pd(PPh₃)₄ are used. Typically, diluents such as DME or a mixture of Toluene/EtOH and basic auxiliaries such as Na₂CO₃ are used.

The Sonogashira coupling according to process b) can be effected according to conventional methods. Typically, Palladium catalysts such as Pd(Ph₃)₂Cl₂ in the presence of CuI are used. Typically, diluents such as TEA are used. A so obtained compound of formula (I) contains a C—C triple bond and can be converted into another compound of formula (I) having a double bond or single bond be a reduction reaction. Such reductions may be done using hydrogen and a heterogenous catalyst, such as Pd or Pt catalysts, optionally on a support.

Starting materials of formula (II) are known or obtainable by known methods. Selected compounds of formula (II) are novel and subject to this invention. Such compounds of formula (II) are useful for the manufacture of compounds of formula (I) and also show interesting pharmaceutical properties.

In a further aspect, the invention provides compounds of formula (II-A)

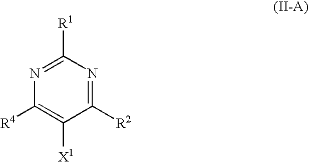

wherein
R¹ is as defined above,
R² represents halogen, hydroxy or substituted amino, the substitutents being selected from the group consisting of hydrogen, alkyl, cycloalkyl;
R⁴ is as defined above
X¹ represents I or Br.

The compounds of formula (II-A) are obtainable by subjecting a compound of formula (V)

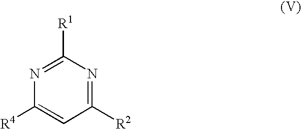

wherein R¹, R² and R⁴ are as defined above, to a conventional bromination or iodination reaction.

Starting materials of formula (III), (IV) and (V) are known or obtainable by known methods.

The examples given in this specification further illustrate the manufacturing processes for compounds of formula (I) and their respective intermediates.

The following considerations apply to the individual reaction steps described above:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.
b) Acid addition salts may be produced from the free bases in known manner, and vice-versa. Compounds of formula (I) in optically pure form can be obtained from the corresponding racemates according to well-known procedures, e.g. HPLC with chiral matrix. Alternatively, optically pure starting materials can be used.
c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar pro-cedures. This separation may take place either at the level of a starting compound or in a compound of formula I itself. Enantiomers may be separated through the formation of dia-stereomeric salts, for example by salt formation with an enantiomer-pure chiral acid, or by means of chromatography, for example by HPLC, using chromatographic substrates with chiral ligands.
d) Suitable diluents for carrying out the above-described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethyelene glycol monomethyl ether, diethylene glycol monoethyl ether. Further, mixtures of diluents may be employed. Depending on the starting materials, reaction conditions and auxiliaries, water or diluents containing water may be suitable. It is also possible to use one a starting material as diluent simultaneously.
e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.
f) The reactions are generally carried out under atmospheric pressure. However, it is also possible to carry out the processes according to the invention under elevated or reduced pressure—in general between 0.1 bar and 10 bar.
g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.
h) Work-up is carried out by customary methods (cf. the Preparation Examples).

The reactions can in each case optionally be followed by reduction, oxidation or functionalisation of the resulting compound and/or by cleavage of protecting groups optionally present, and of recovering the so obtainable compound of the formula I in free base form or in acid addition salt form.

The reactions can be effected according to conventional methods, for example as described in the Examples.

The working-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, which processes are further aspects of the invention, e.g. as described in the Examples.

The starting materials are known or may be prepared according to conventional procedures starting from known compounds, for example as described in the Examples.

Compounds of the formula I and their pharmaceutically acceptable acid addition salts, hereinafter referred to as “agents of the invention”, exhibit valuable pharmacological properties when tested in vitro and in animals, and are therefore useful as medicaments.

In particular, compounds of formula (I) have valuable GABA_{B}-positive modulatory properties. In particular, the agents of the invention act as positive GABA_{B} receptor modulators.

In the functional GTP(γ)³⁵S assay (Lorenzen A, Fuss M, Vogt H, Schwabe U. Measurement of guanine nucleotide-binding protein activation by A₁ adenosine receptor agonists in bovine brain membranes. Stimulation of guanosine-5′-O-(3-[³⁵S]thio)triphosphate binding. Mol. Pharmacol. 1993; 44:115-123) the agents of the invention enhance the GABA-induced GTP( [symbol] )³⁵S binding at recombinant GABA_{B} receptors with EC₅₀ values of about 0.1 μM to about 50 μM.

The agents of the invention are therefore useful for the treatment of any pathology, disorder or clinical condition involving GABA_{B} agonism in their etiology, including psychiatric disorders (such as anxiety, depression, schizophrenia, attention deficit and cognitive disorders, bipolar disorders, social withdrawal), sleep disturbances, drug abuse (e.g. ethanol, opiates, nicotine, cocaine, heroin) and withdrawal, pain (e.g. neuropathic pain), pruritus, convulsive states (such as epilepsy) and spasticity.

The anxiolytic activity of the agents of the invention is confirmed in conventional in vivo assays, including the elevated plus maze model, the Vogel conflict paradigm and the social interaction test in rats.

The elevated plus maze experiments are performed according to the method of Handley and Mithani, Naunyn Schmiedeberg's Arch. Pharmacol. 1984, 327:1-5. At doses of about 1 to about 30 mg/kg p.o., the agents of the invention significantly increase the number of open arm entries versus the number of total arm entries as compared to vehicle.

The Vogel conflict paradigm follows the method described by Vogel et al., Psycho-pharmacologia 1971, 21: 1-7. At doses of about 10 to about 100 mg/kg po the agents of the invention significantly increase the number of shocks accepted by the animals (punished drinking).

The social interaction test is performed according to the method of Vassout et al., Regulatory Peptides, 2000, 96:7-16. At doses of about 1 to about 30 mg/kg p.o., the agents of the invention significantly increase the duration of the social contacts of the intruder towards the resident rat, as compared to the vehicle-treated group.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 1 to about 50 mg/kg animal body weight. In larger mammals, for example humans, an indicated daily dosage is in the range from about 10 to about 2000, preferably from about 10 to about 200 mg of an agent of the invention conveniently administered, for example, in divided doses up to four times a day or in sustained release form.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100 mg/kg animal body weight. In larger mammals, for example humans, an indicated daily dosage is in the range from about 5 to about 500 mg of an agent of the invention, conveniently administered, for example, in divided doses up to four times a day or in sustained release form.

The agents of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a pharmaceutical, e.g. for the treatment of cerebral insufficiency, depression, anxiety and epilepsy.

The present invention furthermore provides a pharmaceutical composition comprising an agent of the invention in association with at least one pharmaceutical carrier or diluent. Such compositions may be manufactured in conventional manner. Unit dosage forms contain, for example, from about 0.25 to about 150, preferably from 0.25 to about 25 mg of a compound according to the invention.

Moreover the present invention provides the use of an agent of the invention, for the manufacture of a medicament for the treatment of any condition mentioned above, e.g. epilepsy, cerebral insufficiency, depression and anxiety.

In still a further aspect the present invention provides a method for the treatment of any condition mentioned above, e.g. epilepsy of the “petit mal” type, cerebral insufficiency, depression and anxiety, in a subject in need of such treatment, which comprises administering to such subject a therapeutically effective amount of an agent of the invention.

The agents of the invention are therefore useful in the treatment of nervous system disorders mediated full or in part by GABA B.

Nervous system disorders mediated full or in part by GABA B are for example acute, traumatic and chronic degenerative processes of the nervous system, such as Parkinson's disease, senile dementia, Alzheimer's disease, Huntington's chorea, amyotrophic lateral sclerosis and multiple sclerosis, psychiatric diseases such as schizophrenia and anxiety, depression, pain, itch, disorders of the eye, GI tract disorders, skin disorders and drug abuse. Anxiety related disorders includes panic disorders, social anxiety, obsessive compulsive disorders (OCD), post traumatic stress disorders (ATSD), generalized anxiety disorders (GAD), phobias.

In accordance with the foregoing, the present invention also provides an agent of the invention for use as a pharmaceutical, e.g. in the treatment of nervous system disorders mediated full or in part by GABA B.

The invention also provides the use of an agent of the invention, in the treatment of nervous system disorders mediated full or in part by GABA B.

Furthermore the invention provides the use of an agent of the invention for the manufacture of a pharmaceutical composition designed for the treatment of nervous system disorders mediated full or in part by GABA B.

In a further aspect the invention relates to a method of treating disorders mediated full or in part by GABA B, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

The preferred agents of the invention include

Further, properly isotope-labeled agents of the invention exhibit valuable properties as histopathological labeling agents, imaging agents and/or biomarkers, hereinafter “markers”, for the selective labeling of the GABA B receptor. More particularly the agents of the invention are useful as markers for labeling the GABA B receptors in vitro or in vivo. In particular, compounds of the invention which are properly isotopically labeled are useful as PET markers. Such PET markers are labeled with one or more atoms selected from the group consisting of ¹¹C, ¹³N, ¹⁵O, ¹⁵F.

The agents of the invention are therefore useful, for instance, for determining the levels of receptor occupancy of a drug acting at the GABA B receptor, or diagnostic purposes for diseases resulting from an imbalance or dysfunction of GABA B receptors, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving GABA B receptors in vivo and in vitro comprising an agent of the invention.

In still a further aspect, the present invention provides a method for labeling brain and peripheral nervous system structures involving GABA B receptors in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following non-limiting Examples illustrate the invention. A list of Abbreviations used is given below.

### AcOEt: Ethyl Acetate

BuMelm BF₄⁻: 1-Butyl-3-methylimidazolium tetrafluoroborate

### DCM: Dichloromethane

### DEA: Diethyl Aniline

### DMF: N,N-dimethylformamide

### HPLC: High Performance Liquid Chromatography

### M.P.: Melting Point

m: multiplet
q: quadruplet
quint: quintuplet

### RT: Room Temperature

s: singulet
sext: sextuplet

### TEA: Triethylamine

### TES: Triethylsilane

Tᵣ: retention time

### EXAMPLE 1

### 6-fluoro-2-(methylthio)-5-(trifluoromethyl)pyrimidin-4(3H)-one

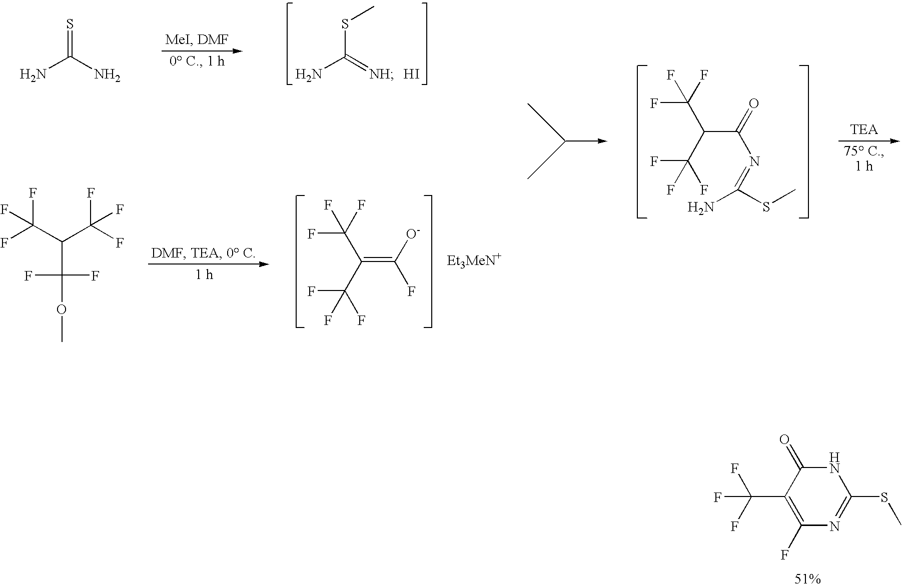

1.51 g of thiourea (19.8 mmoles, 1 eq.) were dissolved in 3 mL of DMF and the solution was cooled to 0° C. 1.23 mL (19.8 mmoles, 1.0 eq.) of iodomethane were added dropwise and the mixture was stirred at 0° C. for 1 h. Also, a solution of 3.02 mL of 1,1,3,3,3-pentafluoro-2-(trifluoromethyl)propyl ether (19.4 mmoles, 0.98 eq.) in 10 mL of DMF was prepared and cooled to 0° C. Then, 5.44 mL (38.9 mmoles, 2.0 eq.) of TEA were added at such a rate that the temperature does not rise above 20° C. After completion of the addition, the solution was stirred at 0° C. for 1 h. Then the solution of methyl imidothiocarbamate hydroiodide was added at such a rate that the temperature does not rise above 25° C. The ice bath was removed and the mixture was stirred at RT for 1 h. The solution was heated at 45° C., the oil bath was removed and 5.44 mL (38.9 mmoles, 2.0 eq.) of TEA were added at such a rate that the temperature was kept below 70° C. The mixture was heated at 75° C. for 1 h. The solution was poured into water and the resulting precipitate was collected by filtration. The solid was dried at 50° C. under vacuum to give 3.27 g of a brown solid which was recrystallized in 30 mL of benzene to afford 2.30 g of a white solid.

Yield: 51%

M.P.: 192-193° C.

LC-MS: Tᵣ=3.89 min. (purity: 100%) (No ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (DMSO-D₆, 400 MHz) δ: 2.55 (s, 31-1).

¹³C-NMR (DMSO-D₆, 100 MHz) δ: 13.5; 122.9; 161.0; 164.5; 167.1; 169.7.

¹⁹F-NMR (DMSO-D₆, 377 MHz) δ: −39.8; −35.8.

### N,N′-dicyclopentyl-2-(methylthio)-5-(trifluoromethyl)pyrimidine-4,6-diamine trifluoroacetate

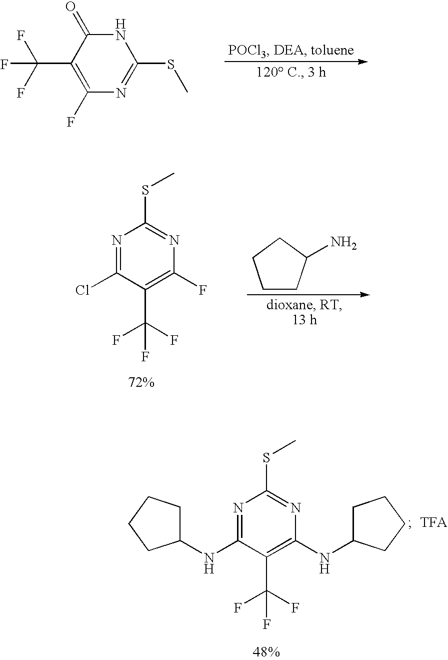

1 g (4.34 mmoles, 1.0 eq.) of 6-fluoro-2-(methylthio)-5-(trifluoromethyl)pyrimidin-4(3H)-one was dissolved in 9 mL of toluene. 1.10 mL (11.4 mmoles, 2.6 eq.) of DEA were added and a solution of 1.10 mL (12.1 mmoles, 2.75 eq.) of POCl₃ in 3.5 mL of toluene was added dropwise. The resulting mixture was heated at 120° C. for 3 h. The solution was cooled to RT and was poured onto 45 mL of iced water. The aqueous phase was extracted once with 30 mL of AcOEt and three more times with 25 mL of AcOEt. The combined organic layers were washed two times with 15 mL of water and once with 15 mL of brine. The organic phase was dried over Na₂SO₄, filtered and evaporated to dryness. The crude compound was purified by flash chromatography on silica gel to give 780 mg of a yellow oil. This compound was used in the next step without further purifications.

Yield: 72%

LC-MS: Tᵣ=2.43 min. (80.8%) and Tᵣ=2.74 min. (19.2%) (no ionization) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

100 mg (0.41 mmoles, 1 eq.) of 4-chloro-6-fluoro-2-(methylthio)-5-(trifluoromethyl)-pyrimidine were dissolved in 1.6 mL of dioxane. The mixture was cooled to 0° C. and a solution of 320 μL (3.24 mmoles, 8.0 eq.) of cyclopentylamine in 700 μL of dioxane was added dropwise. The solution was allowed to reach RT and was stirred at RT for 13 h. Dioxane was removed under reduced pressure and the crude compound was purified by preparative HPLC [Column: Macherey-Nagel, VP 125/21 Nucleodur 100-7 C-18 ec, 21×125 mm, 7 μm, gradient CH₃CN/H₂O/TFA 0.05%: 50-100% CH₃CN (15 min.), 100% CH₃CN (6 min.)] to afford 93 mg of a colourless oil.

Yield: 48%

LC-MS: Tᵣ=6.23 min. (100%) (ES-MS: m/z 361.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CD₃OD, 400 MHz) δ: 1.46-1.81 (m, 12H); 2.01-2.10 (m, 4H); 2.50 (s, 3H); 4.46 (quint, J=6.3 Hz, 2H).

¹⁹F-NMR (CD₃OD, 377 MHz) δ: −55.3; −34.3.

### EXAMPLE 2

### 6-chloro-N-cyclopentyl-2-(methylthio)-5-(trifluoromethyl)pyrimidin-4-amine

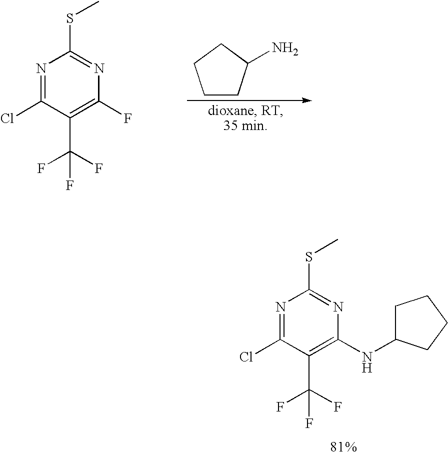

100 mg (0.41 mmoles, 1 eq.) of 4-chloro-6-fluoro-2-(methylthio)-5-(trifluoromethyl)-pyrimidine were dissolved in 1.6 mL of dioxane. The mixture was cooled to 0° C. and a solution of 84 μL (3.24 mmoles, 8.0 eq.) of cyclopentylamine in 700 μL of dioxane was added dropwise. The solution was allowed to reach RT and was stirred at RT for 35 min. Dioxane was removed under reduced pressure and the crude compound was purified by flash chromatography on silica gel to give 102 mg of a colourless oil.

Yield: 81%

LC-MS: Tᵣ=4.41 min. (100%) (ES-MS: m/z 312.0 (M+H); 314.0 (M+2+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 400 MHz) δ: 1.47-1.54 (m, 2H); 1.64-1.81 (m, 4H); 2.09-2.17 (m, 2H); 2.55 (s, 3H); 4.47 (sext, J=6.3 Hz, 1H); 5.75 (m, 1H).

¹⁹F-NMR (CDCl₃, 377 MHz) δ: −54.5.

### EXAMPLE 3

### 6-chloro-2-(methylthio)pyrimidin-4-ol

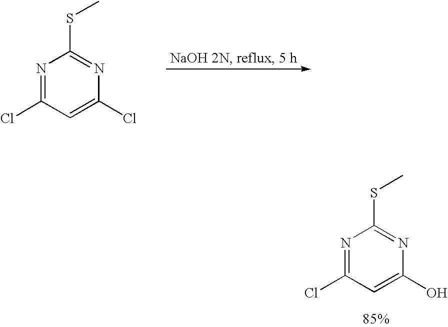

10 g 4,6-dichloro-2-methylthiopyrimidine(51.3 mmoles, 1.0 eq.) were suspended in 250 mL of a 2N aqueous solution of NaOH. The mixture was stirred at 120° C. for 5 h. The solution was cooled to RT and AcOH was added until pH=6 was reached. A white solid has appeared and was filtered off and washed with water. The solid was triturated with 200 mL of Et₂O, filtered and dried under vacuum to give 7.67 g of a white solid.

Yield: 85%

LC-MS: Tᵣ=2.72 min. (85%) (ES-MS: m/z 177.0 (M+H); 179.0 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (DMSO-D₆, 400 MHz) δ: 2.45 (s, 3H); 6.13 (s, 1H).

¹³C-NMR (DMSO-D₆, 400 MHz) δ: 13.5; 106.1; 157.4; 167.4.

### 6-chloro-5-iodo-2-(methylthio)pyrimidin-4-ol

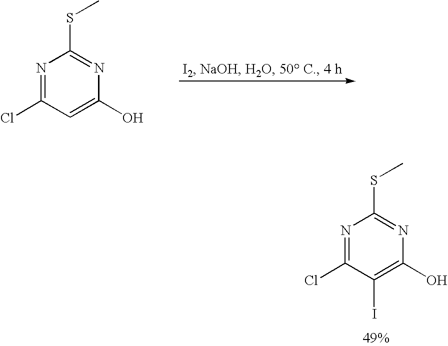

4 g (22.6 mmoles, 1.0 eq.) of 6-chloro-2-(methylthio)pyrimidin-4-ol and 1.09 g (27.2 mmoles, 1.2 eq.) of NaOH were placed into a 500 mL flask. 156 mL of water were added and the solution was stirred at RT until the solid was completely dissolved. 6.78 g (26.7 mmoles, 1.18 eq.) of iodine were added and the solution was heated at 50° C. for 4 h. The solid was filtered off and recrystallized with 200 mL of EtOH. The solution was evaporated to the half and cooled to 0° C. for 1 h. The solid was collected by filtration (First fraction). The solution was evaporated to approximately 50 mL and was cooled to 0° C. for 1 h. The solid was filtered off (Second Fraction). The aqueous layer was evaporated to approximately 40 mL. The precipitate was filtered off and triturated with Et₂O to remove the yellow colour (Third fraction). All fractions were combined to give 3.34 g of a white solid.

Yield: 49%

LC-MS: Tᵣ=3.93 min. (85%) (No ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (DMSO-D₆, 400 MHz) δ: 2.29 (S, 3H).

### 4,6-dichloro-5-iodo-2-(methylthio)pyrimidine

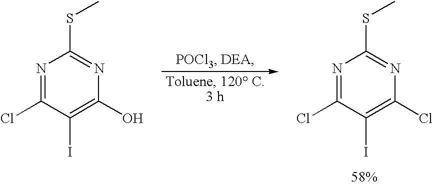

1 g (3.31 mmoles, 1.0 eq.) of 6-chloro-5-iodo-2-(methylthio)pyrimidin-4-ol was poured into 6.8 mL of toluene. This suspension was stirred and 832 μL (8.59 mmoles, 2.60 eq.) of DEA were added. A solution of 832 μL (9.09 mmoles, 2.75 eq.) of POCl₃ in 2.6 mL of toluene was added drop wise. After addition, the mixture was heated to 120° C. for 3 h 15. The crude mixture was poured into 34 mL of iced water and extracted with 25 mL of AcOEt. The aqueous layer was extracted three more times with 20 mL of AcOEt. The combined organic layers were washed two times with 10 mL of water and once with 10 mL of brine. The organic layer was dried over Na₂SO₄, filtered and evaporated to dryness. The crude compound was purified by flash chromatography on silica gel to give 614 mg of a beige solid.

Yield: 58%

M.P.: 98-100° C.

LC-MS: Tᵣ=6.24 min. (100%) (ES-MS: m/z 320.8 (M), 322.8 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 2.58 (s, 3H).

¹³C-NMR (CDCl₃, 400 MHz) δ: 14.5; 90.3; 165.8; 172.6.

### 6-chloro-N-cyclopentyl-5-iodo-2-(methylthio)pyrimidin-4-amine

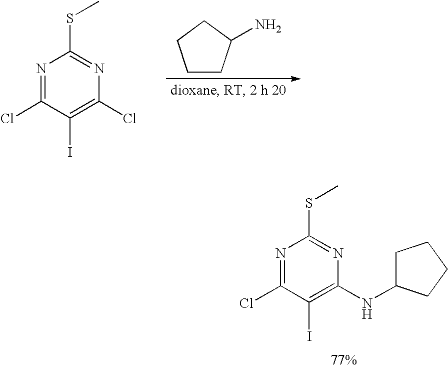

200 mg (0.62 mmoles, 1.0 eq.) of 4,6-dichloro-5-iodo-2-(methylthio)pyrimidine were dissolved in 2 mL of dioxane. The mixture was cooled to 0° C. and a solution of 259 μL (2.62 mmoles, 4.2 eq.) of cyclopentylamine in 1 mL of dioxane was added dropwise. The solution was allowed to reach RT and was stirred for 2 h 20. Solvents were evaporated under reduced pressure and the residue was purified by flash chromatography on silica gel to give 178 mg of a yellow oil.

Yield: 77%

LC-MS: Tᵣ=7.21 min. (100%) (ES-MS: m/z 370.0 (M)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.47-1.55 (m, 2H); 1.66-1.81 (m, 4H); 2.08-2.16 (m, 2H); 2.53 (s, 3H); 4.38 (sext, J=7.4 Hz, 1H); 5.51 (d, J=5.3 Hz, 1H).

¹³C-NMR (CDCl₃, 100 MHz) δ: 14.4; 24.3; 33.4; 54.4; 73.1; 161.0; 161.6; 171.5.

### N,N′-dicyclopentyl-5-iodo-2-(methylthio)pyrimidine-4,6-diamine

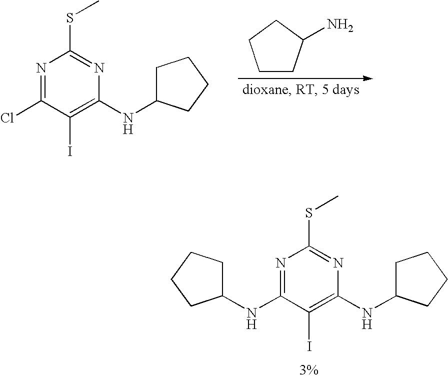

78 mg (0.21 mmoles, 1.0 eq.) of 6-chloro-N-cyclopentyl-5-iodo-2-(methylthio)pyrimidin-4-amine were dissolved in 800 μL of dioxane. The mixture was cooled to 0° C. and a solution of 83 μL (0.84 mmoles, 4.0 eq.) of cyclopentylamine in 400 μL of dioxane was added. The solution was allowed to reach RT and was stirred for 5 days. Solvents were removed under reduced pressure and the crude compound was purified by preparative HPLC (column: Macherey-Nagel, VP 125/21 Nucleodur 100-7 C18 ec, 21×125 mm, 7 μM; gradient CH₃CN/H₂O/0.05% TFA: 50%-100% of acetonitrile in 15 min, 100% of acetonitrile during 6 min.) to give 3.4 mg of a yellow oil.

Yield: 3%

LC-MS: Tᵣ=5.12 min. (100%) (ES-MS: m/z 351.0 (M-cyclopentyl+H); 419.0 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CD₃OD, 400 MHz) δ: 1.52-1.85 (m, 12H); 2.05-2.12 (m, 4H); 2.53 (s, 2.5H); 2.64 (s, 0.5H); 4.39 (quint, J=6.3 Hz, 2H).

### EXAMPLE 4

### 2-(trifluoromethyl)pyrimidine-4,6-diol

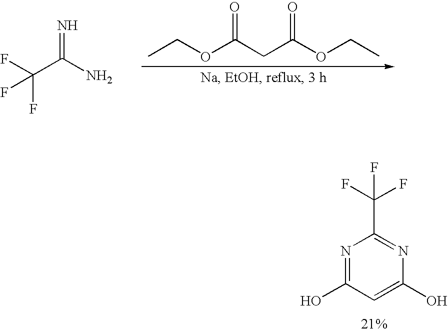

4.11 g of sodium (178.5 mmoles, 1.05 eq.) were added portionwise to 140 mL of EtOH. After completion of the reaction, 31 mL (204 mmoles, 1.20 eq.) of diethylmalonate were added followed by 15 mL (170.0 mmoles, 1.0 eq.) of 2,2,2-trifluoroacetamide. The mixture was refluxed for 3 h. Solvents were removed under reduced pressure and the crude mixture was poured into 115 mL of water. The resulting solution was acidified with aqueous HCl 6N. The resulting precipitate was filtered off, triturated with 50 mL of benzene and dried under vacuum at 40° C. to give 6.5 g of a white solid.

Yield: 21%

LC-MS: Tᵣ=2.82 min. (88%) (ES-MS: m/z 181.0 (M+H)) [Column: Nucleosil C-18HD, 4×70 mm, 3 μm, gradient CH₃CN/H₂O/TFA 0.05%: 5-100% CH₃CN (6 min.), 100% CH₃CN (1.5 min.), flow: 1 mL/min].

¹H-NMR (DMSO-D₆, 400 MHz) δ: 6.00 (s, 1H).

¹³C-NMR (DMSO-D₆, 100 MHz) δ: 90.6; 120.0 (q, J=259.8 Hz); 154.8 (q, J=32.5 Hz); 172.2.

¹⁹F-NMR (DMSO-D₆, 377 MHz) δ: −48.6.

### 6-chloro-5-nitro-2-(trifluoromethyl)pyrimidin-4-ol

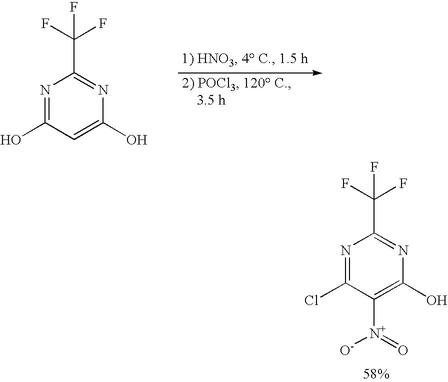

5 mL (122.2 mmoles, 11 eq.) of fuming HNO₃ were cooled at +4° C. (int. T°). Then 2 g (11.11 mmoles, 1.0 eq.) 2-(trifluoromethyl)pyrimidine-4,6-diol of were added portionwise in order to maintain the temperature between +4 and +6° C. After completion of the addition, the solution was stirred at +4° C. for 1.5 h. The mixture was poured into 25 mL of iced water and the aqueous solution was stirred for 20 min. The solution was then evaporated under reduced pressure to dryness. The resulting solid was dried under vacuum overnight to give 2.72 g of a yellow solid. This compound was used in the next step without further purifications.

LC-MS: Tᵣ=2.10 min. (95.7%) (no ionization) [Column: Nucleosil C-18HD, 4×70 mm, 3 μm, gradient CH₃CN/H₂O/TFA 0.05%: 0-100% CH₃CN (6 min.), 100% CH₃CN (1.5 min.), flow: 1 mL/min].

¹³C-NMR (DMSO-D₆, 100 MHz) δ: 118.7 (q, J=258.1 Hz); 120.6; 151.9 (q, J=32.2 Hz); 162.6.

2.72 g of 5-nitro-2-(trifluoromethyl)pyrimidine-4,6-diol were suspended in 20 mL of POCl₃. The mixture was heated at 120° C. for 3.5 h. The solution was cooled to T and the excess of POCl₃ was removed under reduced pressure. The resulting oily residue was poured onto 47 g of crushed ice and the resulting aqueous phase was extracted three times with 100 mL of AcOEt. The combined organic layers were dried over Na₂SO₄, filtered and evaporated to dryness. The crude compound was purified by sublimation (P=20 mBar) to give 1.57 g of a slightly yellow solid.

Yield: 58%

LC-MS: Tᵣ=3.46 min. (94%) (no ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹³C-NMR (DMSO-D₆, 100 MHz) δ: 119.3 (q, J=266.3 Hz); 136.3; 147.9; 154.5 (q, J=39.0 Hz); 162.9; 163.2.

### 6-(cyclopentylamino)-5-nitro-2-(trifluoromethyl)pyrimidin-4-ol

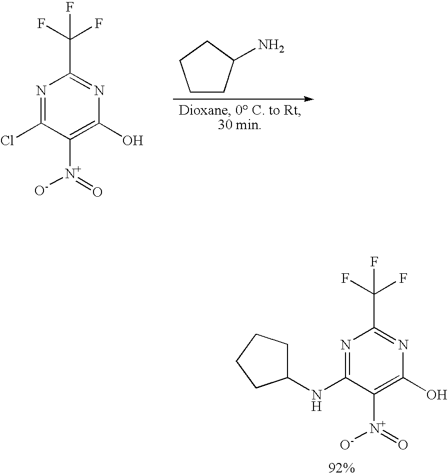

200 mg (0.82 mmoles, 1.0 eq.) of 6-chloro-5-nitro-2-(trifluoromethyl)pyrimidin-4-ol were dissolved in 3 mL of dioxane. The solution was cooled to 0° C. and a solution of 325 μL (3.28 mmoles, 4.0 eq.) of cyclopentylamine in 1.3 mL of dioxane was added dropwise. The reaction mixture was allowed to reach RT and was stirred for 0.5 h. Solvents were removed under reduced pressure and the residue was recrystallized in 4.5 mL of EtOH. The clear solution was allowed to cool to RT and it was left one night in the fridge. The resulting solid was filtered off to afford 220 mg of a yellow solid.

Yield: 92%

M.P.: decomposition LC-MS: Tᵣ=4.66 min. (100%) (ES-MS: m/z 293.0 (M+H); 315.0 (M+Na)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (DMSO-D₆, 400 MHz) δ: 1.45 (m, 6H); 1.88-2.04 (m, 2H); 3.47-3.55 (m, 0.5H); 4.38 (sext., J=6.8 Hz, 0.5H); 7.79 (s, 1.5H); 8.80 (d, J=7.4 Hz, 0.5H).

¹⁹F-NMR (DMSO-D₆, 377 MHz) δ: −71.4.

### EXAMPLE 5

### 4,6-dichloro-5-nitro-2-(trifluoromethyl)pyrimidine

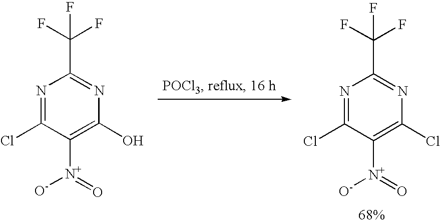

1.14 g (4.68 mmoles, 1.0 eq.) of 6-chloro-5-nitro-2-(trifluoromethyl)pyrimidin-4-ol were suspended in 11.5 mL of POCl₃, one drop of DMF was added and the mixture was heated at 120° C. for 16 h. The solution was cooled to RT and the excess of POCl₃ was removed under reduced pressure. The oily residue was poured onto ice and the resulting aqueous phase was extracted three times with 20 mL of AcOEt. The organic phase was washed once with 20 mL of brine, dried over Na₂SO₄, filtered and evaporated to dryness. The crude compound was purified by flash chromatography on silica gel to give 828 mg of a colourless liquid.

Yield: 68%

LC-MS: Tᵣ=5.47 min. (100%) (no ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹³C-NMR (CDCl₃, 100 MHz) δ: 117.7 (q, J=264.1 Hz); 119.0; 154.7; 155.4 (q, J=52.8).

¹⁹F-NMR (CDCl₃, 377 MHz) δ: −70.7.

### N,N′-dicyclopentyl-5-nitro-2-(trifluoromethyl)pyrimidine-4,6-diamine

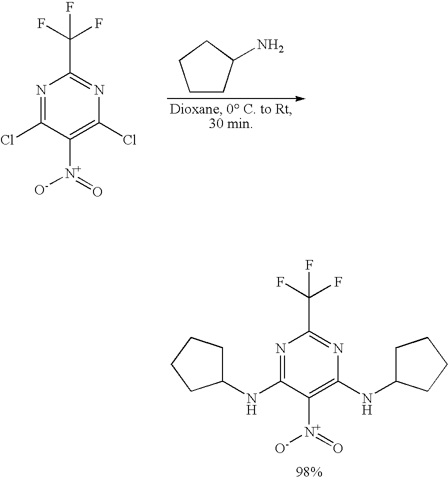

200 mg (0.76 mmoles, 1.0 eq.) of 4,6-dichloro-5-nitro-2-(trifluoromethyl)pyrimidine were dissolved in 3 mL of dioxane. The solution was cooled to 0° C. and a solution of 603 μL (6.11 mmoles, 8.0 eq.) of cyclopentylamine in 1.3 mL of dioxane was added dropwise. The solution was allowed to reach RT and was stirred for 0.5 h. Solvents were removed under reduced pressure and the crude compound was purified by flash chromatography on silica gel to give 268 mg of a yellow oil.

Yield: 98%

LC-MS: Tᵣ=5.96 min. (100%) (ES-MS: m/z 360.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CD₃OD, 400 MHz) δ: 1.58-1.87 (m, 12H); 2.10-2.20 (m, 4H); 4.62 (quint, J=5.8 Hz, 2H).

¹³C-NMR (CDCl₃, 100 MHz) δ: 19.4; 28.4; 48.7; 107.4; 114.2 (q, J=266.3 Hz); 151.9 (q, J=32.5 Hz); 152.6.

¹⁹F-NMR (CD₃OD, 377 MHz) δ: −74.2.

### EXAMPLE 6

### 4-chloro-2-(trifluoromethyl)pyrimidine

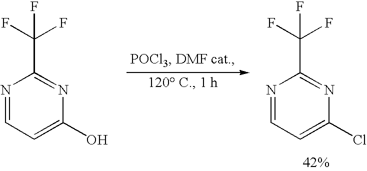

1.5 g (8.59 mmoles, 1.0 eq.) of 4-hydroxy-2-trifluoromethylpyrimidine were dissolved in 15 mL of POCl₃. One drop of DMF was added and the mixture was heated at 120° C. for 1 h. The excess of POCl₃ was removed under reduced pressure and the mixture was poured onto ice. The resulting aqueous phase was extracted three times with 25 mL of AcOEt. The combined organic layers were washed once with 25 mL of brine, dried over Na₂SO₄, filtered and evaporated to dryness. The crude compound was purified by flash chromatography on silica gel to give 656 mg of a colourless liquid.

Yield: 42%

LC-MS: Tᵣ=4.24 min. (100%) (no ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 7.58 (d, J=4.7 Hz, 1H); 8.79 (d, J=4.7 Hz, 1H).

¹³C-NMR (CDCl₃, 100 MHz) δ: 119.0 (q, J=268.8 Hz); 124.3; 157.4 (q, J=32.8 Hz); 159.0; 162.9.

¹⁹F-NMR (CDCl₃, 282 MHz) δ: −71.3.

### N-cyclopentyl-2-(trifluoromethyl)pyrimidin-4-amine

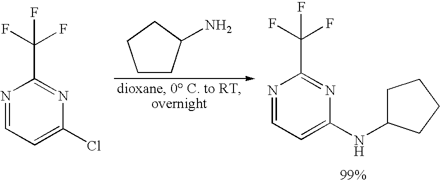

600 mg (3.29 mmoles, 1.0 eq.) of 4-chloro-2-(trifluoromethyl)pyrimidine were dissolved in 13 mL of dioxane. The solution was cooled to 0° C. and a solution of 1.3 mL of cyclopentylamine (13.15 mmoles, 4.0 eq.) in 5 mL of dioxane was added slowly. The mixture was allowed to warm to RT and it was stirred overnight. Solvents were removed under reduced pressure and the residue was purified by flash chromatography on silica gel to give 754 mg of a slightly pink oil.

Yield: 99%

LC-MS: Tᵣ=4.98 min. (100%) (ES-MS: m/z 232.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.50-1.86 (m, 6H); 2.03-2.14 (m, 2H); 3.96 (m, 0.2H); 4.39 (m, 0.8H); 6.56-6.71 (m, 8.03 (m, 0.8H); 8.25 (m, 0.2H).

¹⁹F-NMR (CDCl₃, 282 MHz) δ: −71.9.

### 5-bromo-N-cyclopentyl-2-(trifluoromethyl)pyrimidin-4-amine

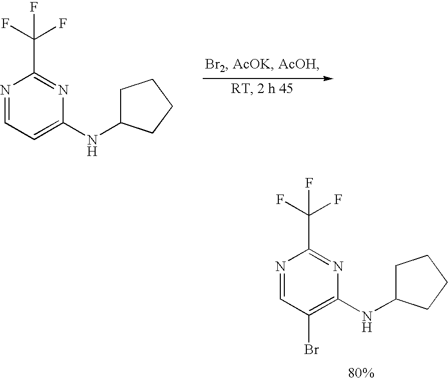

383 mg (1.66 mmoles, 1.0 eq.) of N-cyclopentyl-2-(trifluoromethyl)pyrimidin-4-amine and 211 mg (2.15 mmoles, 1.3 eq.) of AcOK were dissolved in 6.66 mL of AcOH. The solution was cooled to 0° C. and a solution of 102 μL (1.99 mmoles, 1.2 eq.) of bromine in 460 μL of AcOH was added slowly. The mixture was allowed to reach RT and was stirred for 2 h 45. The crude mixture was poured onto 50 mL of a saturated solution of Na₂CO₃. The aqueous phase was extracted four times with 20 mL of AcOEt. The combined organic layers were washed once with 20 mL of brine, dried over Na₂SO₄, filtered and evaporated under reduced pressure. The crude compound was purified by flash chromatography on silica gel to give 414 mg of a colourless oil.

Yield: 80%

LC-MS: Tᵣ=6.21 min. (100%) (ES-MS: m/z 310.0 (M); 312.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.44-1.58 (m, 2H); 1.63-1.83 (m, 4H); 2.10-2.21 (m, 2H); 4.43 (sext, J=6.7 Hz, 1H); 5.56 (d, J=4.7 Hz, 1H); 8.33 (s, 1H).

¹⁹F-NMR (CDCl₃, 282 MHz) δ: −71.6.

### General Procedure for the Suzuki Cross Coupling Reaction:

1 mg (0.006 mmoles, 0.02 eq.) of palladium acetate and 4 mg (0.007 mmoles, 0.03 eq.) of dppf were heated in 743 μL of degassed DME for 15 min. under argon. The solution was cooled to RT and a solution of 90 mg of 5-bromo-N-cyclopentyl-2-(trifluoromethyl)pyrimidin-4-amine in 1.12 mL of DME was added followed by 155 mg (0.58 mmoles, 2.0 eq.) of K₃PO₄. 3H₂O, 0.38 mmoles (1.3 eq.) of the corresponding boronic acid and 558 μL of water. The mixture was heated at 85° C. for 2.5 h under argon. The mixture was allowed to cool to RT and 5 mL of water were added followed by 10 mL of AcOEt. The aqueous phase was extracted 3 more times with 10 mL of AcOEt. The combined organic layers were washed once with 10 mL of brine, dried over Na₂SO₄, filtered and evaporated to dryness. The crude compound was purified by flash chromatography on silica gel to give the desired compound.

### 5-(3-butylphenyl)-N-cyclopentyl-2-(trifluoromethyl)pyrimidin-4-amine

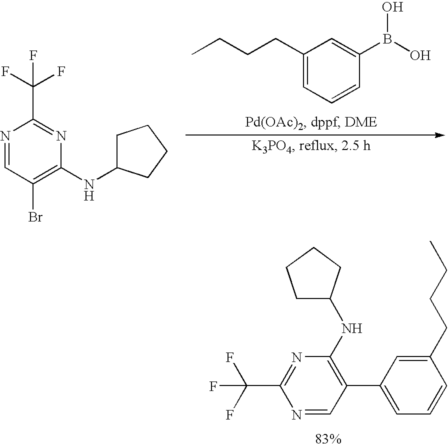

Aspect: colourless oil Mass obtained: 88 mg

Yield: 83%

LC-MS: Tᵣ=5.81 min. (100%) (ES-MS: m/z 364.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 0.94 (t, J=7.1 Hz, 3H); 1.30-1.45 (m, 4H); 1.59-1.70 (m, 6H); 2.04-2.16 (m, 2H); 2.68 (t, J=7.9 Hz, 2H); 4.45 (sext, J=5.9 Hz, 1H); 5.25 (d, J=6.3 Hz, 1H); 7.17-7.20 (m, 2H); 7.28 (d, J=7.9 Hz, 1H); 7.42 (t, J=7.9 Hz, 1H); 8.06 (s, 1H).

¹³C-NMR (CDCl₃, 75 MHz) δ: 14.4; 22.6; 23.9; 33.1; 34.0; 36.1; 53.1; 120.0 (q, J=272.1 Hz); 121.0; 125.9; 128.6; 129.3; 129.9; 133.4; 144.7; 153.1; 155.4 (q, J=34.6 Hz); 160.0.

¹⁹F-NMR (CDCl₃, 282 MHz) δ: −71.8.

### EXAMPLE 7

### N-cyclopentyl-5-(4-ethylphenyl)-2-(trifluoromethyl)pyrimidin-4-amine

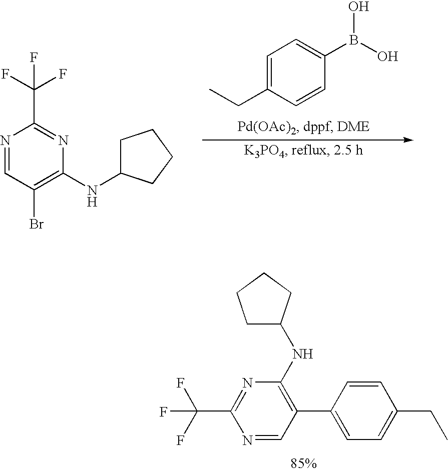

Aspect: colourless oil Mass obtained: 88 mg

Yield: 85%

LC-MS: Tᵣ=4.59 min. (100%) (ES-MS: m/z 336.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.26-1.42 (m, 5H); 1.59-1.70 (m, 4H); 2.05-2.16 (m, 2H); 2.72 (q, J=7.9 Hz, 2H); 4.45 (sext, J=7.1 Hz, 1H); 5.26 (d, J=6.7 Hz, 1H); 7.27 (d, J=7.9 Hz, 2H); 7.34 (d, J=7.9 Hz, 2H); 8.05 (s, 1H).

¹³C-NMR (CDCl₃, 75 MHz) δ: 15.7; 23.9; 28.8; 33.4; 53.1; 120.0 (q, J=277.1 Hz); 121.0; 128.8; 129.8; 130.8; 145.6; 153.1; 155.7 (q, J=30.0 Hz); 160.0.

¹⁹F-NMR (CDCl₃, 282 MHz) δ: −71.8.

### EXAMPLE 8

### N-cyclopentyl-5-(4-methylphenyl)-2-(trifluoromethyl)pyrimidin-4-amine

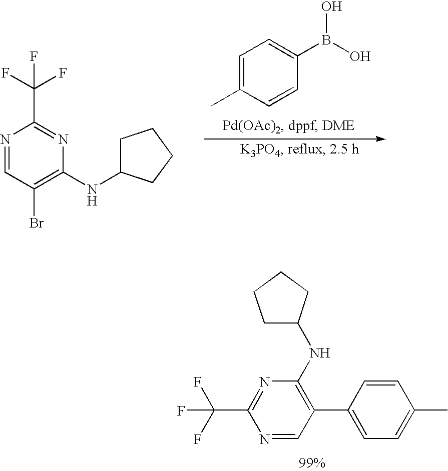

Aspect: colourless oil Mass obtained: 92 mg

Yield: 99%

LC-MS: Tᵣ=4.06 min. (100%) (ES-MS: m/z 322.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.29-1.41 (m, 2H); 1.59-1.68 (m, 4H); 2.04-2.14 (m, 2H); 2.42 (s, 3H); 4.43 (sext, J=6.3 Hz, 1H); 5.24 (d, J=6.3 Hz, 1H); 7.24 (d, J=7.9 Hz, 2H); 7.32 (d, J=7.9 Hz, 2H); 8.03 (s, 1H).

¹³C-NMR (CDCl₃, 75 MHz) δ: 21.6; 23.9; 33.1; 53.1; 120.0 (q, J=277.1 Hz); 121.0; 128.5; 130.5; 130.7; 139.3; 153.1; 155.4 (q, J=34.6 Hz); 160.0.

¹⁹F-NMR (CDCl₃, 282 MHz) δ: −71.3.

### EXAMPLE 9

### N-cyclopentyl-5-(4-methoxyphenyl)-2-(trifluoromethyl)pyrimidin-4-amine

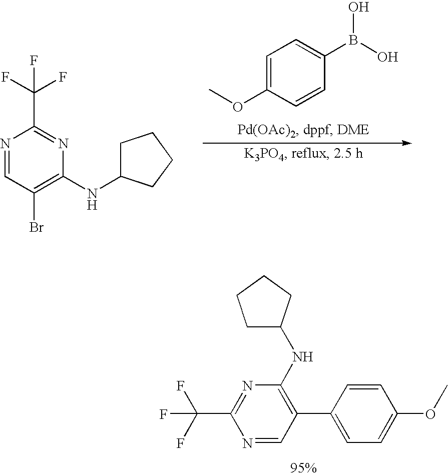

Aspect: colourless oil Mass obtained: 93 mg

Yield: 95%

LC-MS: Tᵣ=3.44 min. (100%) (ES-MS: m/z 338.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.28-1.41 (m, 2H); 1.59-1.70 (m, 4H); 2.04-2.14 (m, 2H); 3.84 (s, 3H); 4.42 (sext, J=7.1 Hz, 1H); 5.21 (d, J=7.1 Hz, 1H); 7.01 (d, J=8.7 Hz, 2H); 7.29 (d, J=8.7 Hz, 2H); 8.01 (s, 1H).

¹³C-NMR (CDCl₃, 75 MHz) δ: 24.3; 33.1; 52.7; 55.7; 115.4; 120.0 (q, J=272.1 Hz); 120.6; 125.2; 129.9; 153.1; 155.4 (q, J=30.0 Hz); 160.1.

¹⁹F-NMR (CDCl₃, 282 MHz) δ: −71.5.

### EXAMPLE 10

### N-cyclopentyl-5-(3-methylphenyl)-2-(trifluoromethyl)pyrimidin-4-amine

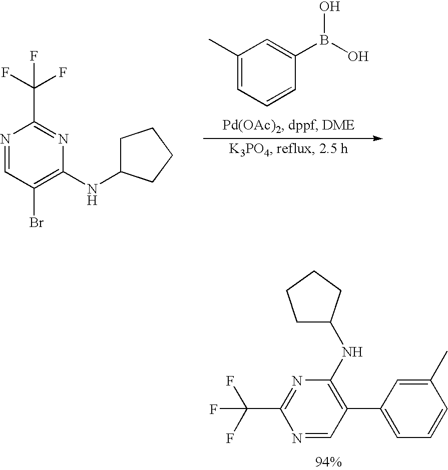

Aspect: colourless oil Mass obtained: 88 mg

Yield: 94%

LC-MS: Tᵣ=4.03 min. (100%) (ES-MS: m/z 338.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.26-1.42 (m, 2H); 1.58-1.71 (m, 4H); 2.04-2.14 (m, 2H); 2.42 (s, 3H); 4.43 (sext, J=7.5 Hz, 1H); 5.24 (d, J=7.5 Hz, 1H); 7.13-7.18 (m, 2H); 7.26 (d, J=7.9 Hz, 1H); 7.39 (t, J=7.9 Hz, 1H); 8.04 (s, 1H).

¹³C-NMR (CDCl₃, 75 MHz) δ: 21.6; 23.9; 33.1; 53.1; 120.0 (q, J=272.1 Hz); 121.0; 125.6; 129.5; 129.8; 130.1; 133.4; 140.0; 153.1; 155.7 (q, J=34.6 Hz); 160.0.

¹⁹F-NMR (CDCl₃, 282 MHz) δ: −71.8.

### EXAMPLE 11

### 5-(4-butylphenyl)-N-cyclopentyl-2-(trifluoromethyl)pyrimidin-4-amine

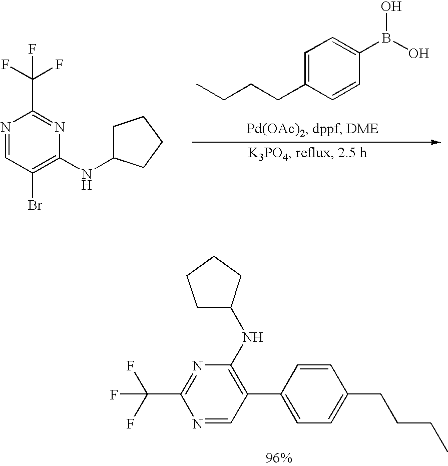

Aspect: colourless oil Mass obtained: 101 mg

Yield: 96%

LC-MS: Tᵣ=5.79 min. (100%) (ES-MS: m/z 364.2 (M+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 0.95 (t, J=7.9 Hz, 3H); 1.29-1.46 (m, 4H); 1.60-1.70 (m, 6H); 2.05-2.16 (m, 2H); 2.67 (t, J=7.9 Hz, 2H); 4.43 (sext, J=7. Hz, 1H); 5.25 (d, J=7.1 Hz, 1H); 7.26 (d, J=8.7 Hz, 2H); 7.31 (d, J=8.7 Hz, 2H); 8.03 (s, 1H).

¹³C-NMR (CDCl₃, 75 MHz) δ: 14.4; 22.6; 24.0; 33.1; 33.8; 35.7; 53.1; 120.0 (q, J=272.1 Hz); 121.0; 128.5; 130.2; 130.7; 144.6; 153.4; 155.4 (q, J=34.6 Hz); 160.1.

¹⁹F-NMR (CDCl₃, 282 MHz) δ: −71.8.

### EXAMPLE 12

### 6-chloro-N-cyclopentyl-2-methylpyrimidin-4-amine

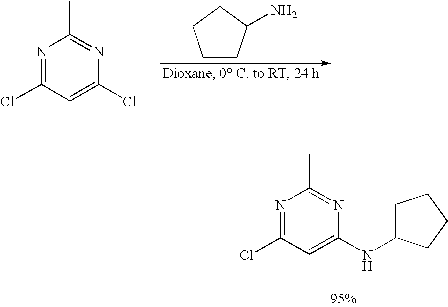

5 g (30.67 mmoles, 1.0 eq.) of 4,6-dichloro-2-methylpyrimidine were dissolved in 121 mL of dioxane. The solution was cooled to 0° C. and a solution of 12.12 mL (122.7 mmoles, 4.0 eq.) of cyclopentylamine in 53 mL of dioxane was added dropwise at 9° C. (int. T°) over 5 min. The solution was allowed to reach RT and was stirred for 24 h. Solvents were removed under reduced pressure and the crude compound was purified by flash chromatography on silica gel to give 6.15 g of an orange oil.

Yield: 95%

LC-MS: Tᵣ=3.29 min. (100%) (ES-MS: m/z 212.2 (M+H); 214.2 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.46-1.55 (m, 2H); 1.63-1.80 (m, 4H); 2.00-2.09 (m, 2H); 2.46 (s, 3H); 3.92 (m, 1H); 5.18 (m, 1H); 6.18 (s, 1H).

### 6-chloro-N-cyclopentyl-5-iodo-2-methylpyrimidin-4-amine

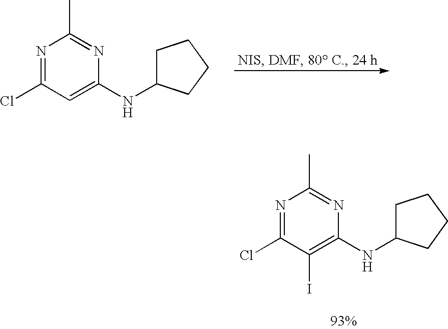

5 g (23.62 mmoles, 1.0 eq.) of 6-chloro-N-cyclopentyl-2-methylpyrimidin-4-amine were dissolved in 37 mL of DMF, then, 5.3 g (70.85 mmoles, 3.0 eq.) of NIS were added. The solution was heated at 80° C. for 1 h. 5.3 g (70.85 mmoles, 3.0 eq.) of NIS were added and it was stirred at 80° C. for additional 1 h. 5.3 g (70.85 mmoles, 3.0 eq.) of NIS were added and the mixture was stirred at 80° C. for additional 22 h. The solution was allowed to cool to RT and 370 mL of water were added. The aqueous phase was extracted five times with 200 mL of AcOEt. The combined organic layers were washed three times with 200 mL of NaOH 0.5N, once with 100 mL of a saturated Na₂CO₃ solution and once with 100 mL of brine. The organic phase was dried over Na₂SO₄, filtered and evaporated to dryness. The crude compound was purified by flash chromatography on silica gel to give 7.43 g of a colourless oil.

Yield: 93%

LC-MS: Tᵣ=6.37 min. (100%) (ES-MS: m/z 338.0 (M+H); 340.0 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (DMSO-D₆, 400 MHz) δ: 1.52-1.62 (m, 4H); 1.68-1.76 (m, 2H); 1.92-2.00 (m, 2H); 2.35 (s, 3H); 4.38 (sext, J=7.4 Hz, 1H); 6.46 (d, J=7.4 Hz, 1H).

¹³C-NMR (CDCl₃, 100 MHz) δ: 23.6; 25.2; 33.1; 53.8; 76.1; 161.6; 167.5.

### General Procedure for the Suzuki Cross-Coupling Reaction Using Pd(OAc)₂:

2 mg (0.009 mmoles, 0.02 eq.) of palladium acetate and 7 mg (0.013 mmoles, 0.03 eq.) of dppf were heated in 1.14 mL of degassed DME for 15 min. under argon. The solution was cooled to RT and a solution of 150 mg (0.44 mmoles, 1.0 eq.) of 6′-chloro-N-cyclopentyl-5-iodo-2-methylpyrimidin-4-amine in 1.7 mL of DME was added followed by 237 mg (0.89 mmoles, 2.0 eq.) of K₃PO₄. 3H₂O, 0.47 mmoles (1.05 eq.) of the corresponding boronic acid and 847 μL of water. The mixture was heated at 85° C. for 2 h under argon. The mixture was allowed to cool to RT and 10 mL of water were added followed by 20 mL of AcOEt. The aqueous phase was extracted 2 more times with 20 mL of AcOEt. The combined organic layers were washed once with 20 mL of brine, dried over Na₂SO₄, filtered and evaporated to dryness. The crude compound was purified by flash chromatography on silica gel to give the desired derivative.

### 5-(4-butylphenyl)-6-chloro-N-cyclopentyl-2-methylpyrimidin-4-amine

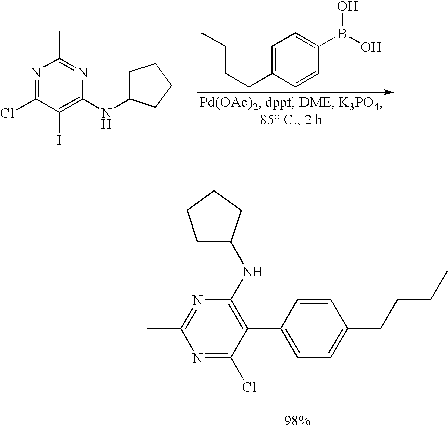

Aspect: white solid Mass obtained: 149 mg

Yield: 98%

M.P.: 80-85° C.

LC-MS: Tᵣ=3.83 min. (100%) (ES-MS: m/z 344.2 (M+H); 346.2 (M+2+H)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.92 (t, J=6.3 Hz, 3H); 1.17-1.29 (m, 2H); 1.37 (sext, J=6.3 Hz, 2H); 1.53-1.67 (m, 6H); 1.92-2.03 (m, 21-0; 2.50 (s, 3H); 2.63 (t, J=7.9 Hz, 2H); 4.34 (sext, J=7.9 Hz, 1H); 4.56 (d, J=7.9 Hz, 1H); 7.12 (d, J=7.9 Hz, 2H); 7.25 (d, J=7.9 Hz, 2H).

¹³C-NMR (CDCl₃, 75 MHz) δ: 14.4; 22.9; 24.1; 26.2; 33.4; 33.8; 35.7; 53.1; 113.4; 129.8; 130.0; 130.2; 143.6; 156.4; 161.3; 166.6.

### General Procedure for the Catalytic Hydrogenation:

The corresponding 6-chloro-N-cyclopentyl-2-methyl-5-phenylpyrimidin-4-amine (1.0 eq.) was dissolved in 2 mL of EtOH. 1.1 eq. of AcONa were added followed by 10% (m/m) of Pd/C 10%. The mixture was hydrogenated at atmospheric Pressure and at RT. When the reaction was completed, the catalyst was removed by filtration and EtOH was removed by evaporation under reduced pressure. The crude compound was then purified by chromatography on silica gel to give the desired compound.

### 5-(4-butylphenyl)-N-cyclopentyl-2-methylpyrimidin-4-amine

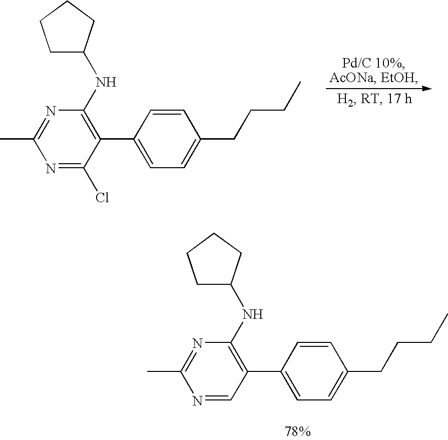

This compound was prepared according to the general procedure described above starting from 50 mg of 5-(4-butylphenyl)-6-chloro-N-cyclopentyl-2-methylpyrimidin-4-amine (0.14 mmoles)

Aspect: colourless oil Mass obtained: 35 mg

Yield: 78%

LC-MS: Tᵣ=4.92 min. (100%) (ES-MS: m/z 310.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 0.96 (t, J=6.3 Hz, 3H); 1.34-1.46 (m, 4H); 1.56-1.71 (m, 6H); 1.96-2.05 (m, 2H); 2.47 (s, 3H); 2.66 (t, J=7.1 Hz, 2H); 4.46 (quint, J=7.1 Hz, 1H); 7.25 (d, J=7.9 Hz, 2H); 7.30 (d, J=7.9 Hz, 2H); 7.74 (s, 1H).

¹³C-NMR (CD₃OD, 75 MHz) δ: 13.4; 22.3; 23.6; 24.6; 32.4; 33.8; 35.4; 52.4; 117.0; 128.5; 129.5; 131.5; 143.3; 151.8; 159.7; 165.9.

### EXAMPLE 13

### 6-chloro-N-cyclopentyl-5-(4-ethylphenyl)-2-methylpyrimidin-4-amine

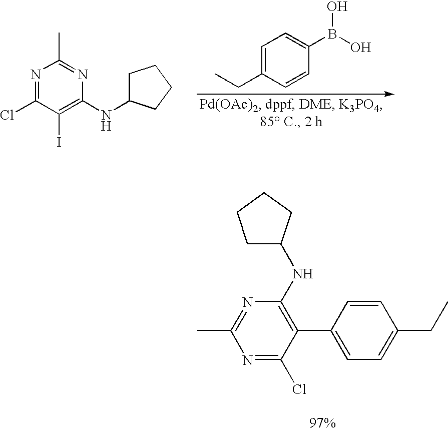

This compound was prepared according to the general procedure described for the example 12.

Aspect: yellow solid Mass obtained: 136 mg

Yield: 97%

M.P.: 72-75° C.

LC-MS: Tᵣ=6.01 min. (100%) (ES-MS: m/z 316.2 (M+H); 318.2 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.18-1.31 (m, 5H); 1.52-1.60 (m, 4H); 1.81-2.05 (m, 2H); 2.51 (s, 3H); 2.71 (q, J=7.9 Hz, 2H); 4.37 (sext, J=7.9 Hz, 1H); 4.58 (d, J=7.9 Hz, 1H); 7.14 (d, J=7.9 Hz, 2H); 7.29 (d, J=7.9 Hz, 2H).

¹³C-NMR (CDCl₃, 75 MHz) δ: 15.2; 23.9; 26.1; 28.7; 32.9; 52.9; 13.2; 129.0; 130.0; 144.8; 156.1; 161.3; 166.8.

### N-cyclopentyl-5-(4-ethylphenyl)-2-methylpyrimidin-4-amine

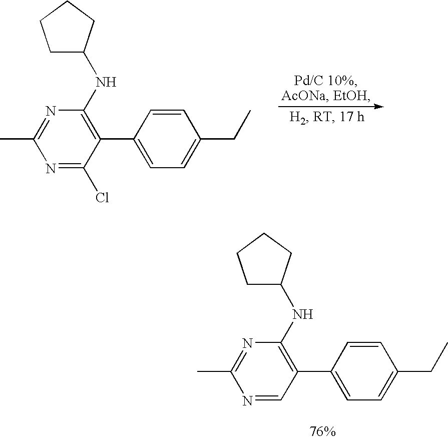

This compound was prepared according to the general procedure described for the example 12 starting from 50 mg (0.16 mmoles) of 6-chloro-N-cyclopentyl-5-(4-ethylphenyl)-2-methylpyrimidin-4-amine.

Aspect: colourless oil Mass obtained: 34 mg

Yield: 76%

LC-MS: Tᵣ=4.26 min. (100%) (ES-MS: m/z 282.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.26 (t, J=7.9 Hz, 3H); 1.33-1.45 (m, 2H); 1.55-1.71 (m, 4H); 1.96-2.05 (m, 2H); 2.46 (s, 3H); 2.68 (q, J=7.9 Hz, 2H); 4.45 (quint, J=7.9 Hz, 1H); 7.25 (d, J=7.9 Hz, 2H); 7.31 (d, J=7.9 Hz, 2H); 7.74 (s, 1H).

¹³C-NMR (CD₃OD, 75 MHz) δ: 15.1; 23.4; 24.3; 28.5; 32.5; 52.5; 117.0; 128.5; 128.8; 131.5; 144.6; 151.8; 159.7; 165.9.

### EXAMPLE 14

### 6-chloro-N-cyclopentyl-2-methyl-5-(4-methylphenyl)pyrimidin-4-amine

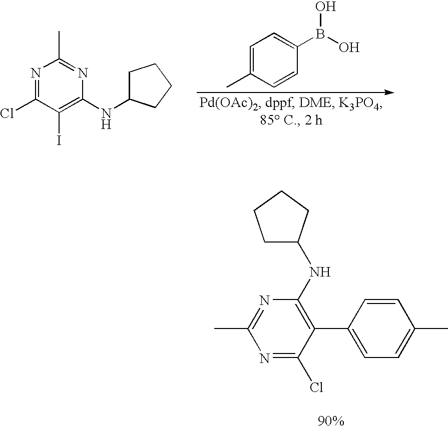

This compound was prepared according to the general procedure described for the example 12.

Aspect: White solid Mass obtained: 121 mg

Yield: 90%

M.P.: 96-100° C.

LC-MS: Tᵣ=5.70 min. (100%) (ES-MS: m/z 302.2 (M); 304.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.17-1.27 (m, 2H); 1.51-1.59 (m, 4H); 1.92-2.03 (m, 2H); 2.38 (s, 3H); 2.50 (s, 3H); 4.34 (sext, J=7.9 Hz, 1H); 4.55 (d, J=7.9 Hz, 1H); 7.10 (d, J=7.9 Hz, 2H); 7.25 (d, J=7.9 Hz, 2H).

¹³C-NMR (CDCl₃, 75 MHz) δ: 21.6; 23.9; 26.2; 33.1; 53.1; 113.4; 129.8; 130.5; 138.7; 156.4; 161.3; 166.5.

### N-cyclopentyl-2-methyl-5-(4-methylphenyl)pyrimidin-4-amine

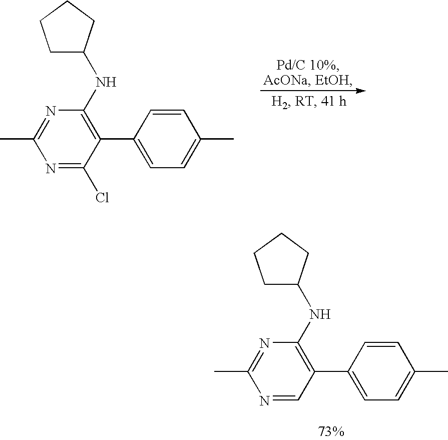

This compound was prepared according to the general procedure described for the example 12 starting from 45 mg (0.16 mmoles) of 6-chloro-N-cyclopentyl-2-methyl-5-(4-methylphenyl)pyrimidin-4-amine.

Aspect: colourless oil Mass obtained: 29 mg

Yield: 73%

LC-MS: Tᵣ=3.95 min. (100%) (ES-MS: m/z 268.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 ml/min].

¹H-NMR (CD₃OD, 400 MHz) δ: 1.38-1.45 (m, 2H); 1.59-1.74 (m, 4H); 2.00-2.08 (m, 2H); 2.42 (s, 3H); 2.50 (s, 3H); 4.49 (quint, J=6.3 Hz, 1H); 7.26 (d, J=7.4 Hz, 2H); 7.34 (d, J=8.4 Hz, 2H); 7.78 (s, 1H).

¹³C-NMR (CD₃OD, 100 MHz) δ: 20.0; 23.2; 23.9; 32.2; 51.9; 116.8; 128.4; 129.7; 130.9; 138.1; 151.6; 159.4; 165.5.

### EXAMPLE 15

### 6-chloro-N-cyclopentyl-5-(4-methoxyphenyl)-2-methylpyrimidin-4-amine

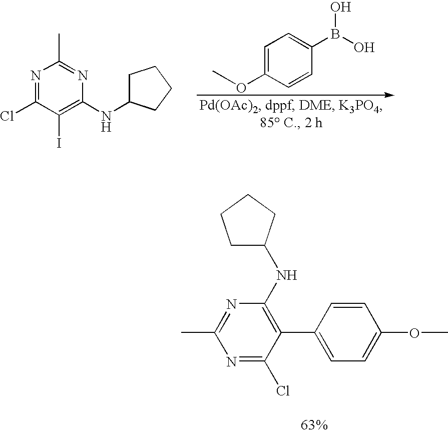

This compound was prepared according to the general procedure described for the example 12.

Aspect: white solid Mass obtained: 89 mg

Yield: 63%

M.P.: 100-107° C.

LC-MS: Tᵣ=5.25 min. (100%) (ES-MS: m/z 318.2 (M); 320.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.24-1.31 (m, 2H); 1.58-1.66 (m, 4H); 2.00-2.08 (m, 2H); 2.54 (s, 3H); 3.88 (s, 3H); 4.39 (sext, J=6.8 Hz, 1H); 4.60 (d, J=6.8 Hz, 1H); 7.03 (d, J=10.5 Hz, 2H); 7.20 (d, J=9.5 Hz, 2H).

¹³C-NMR (CDCl₃, 100 MHz) δ: 23.6; 25.9; 33.1; 52.8; 55.1; 113.1; 115.1; 124.6; 131.1; 156.7; 159.7; 161.3; 166.6.

### N-cyclopentyl-5-(4-methoxyphenyl)-2-methylpyrimidin-4-amine

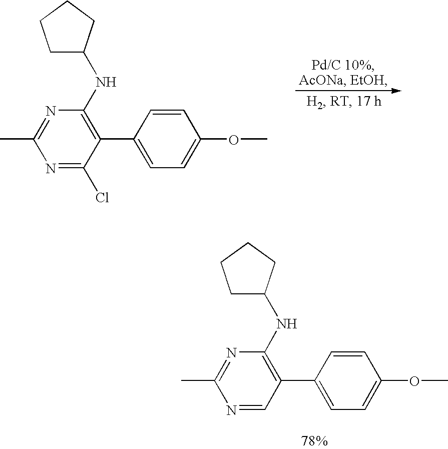

This compound was prepared according to the general procedure described for the example 12 starting from 40 mg (0.13 mmoles) of 6-chloro-N-cyclopentyl-5-(4-methoxyphenyl)-2-methylpyrimidin-4-amine.

Aspect: colourless oil Mass obtained: 28 mg

Yield: 78%

LC-MS: Tᵣ=3.77 min. (100%) (ES-MS: m/z 284.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.34-1.45 (m, 2H); 1.55-1.71 (m, 4H); 1.97-2.06 (m, 2H); 2.46 (s, 3H); 3.83 (s, 3H); 4.45 (quint, J=7.1 Hz, 1H); 7.03 (d, J=8.7 Hz, 2H); 7.26 (d, J=8.7 Hz, 2H); 7.72 (s, 1H).

¹³C-NMR (CD₃OD, 75 MHz) δ: 23.6; 24.3; 32.4; 52.4; 54.7; 114.8; 116.7; 126.2; 129.8; 151.5; 159.7; 160.0; 165.9.

### EXAMPLE 16

### 6-chloro-N-cyclopentyl-2-methyl-5-(3-methylphenyl)pyrimidin-4-amine

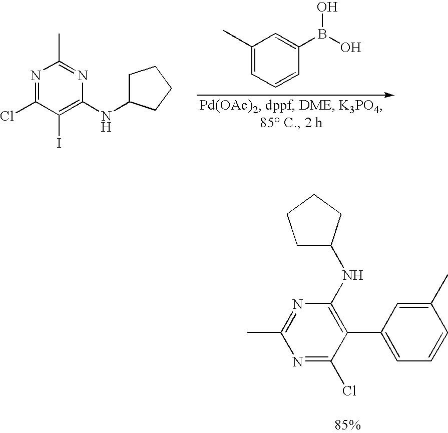

This compound was prepared according to the general procedure described for the example 12.

Aspect: white solid Mass obtained: 114 mg

Yield: 85%

M.P.: 107-109° C.

LC-MS: Tᵣ=5.69 min. (100%) (ES-MS: m/z 302.2 (M); 304.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.22-1.33 (m, 2H); 1.58-1.66 (m, 4H); 2.00-2.08 (m, 2H); 2.42 (s, 3H); 2.55 (s, 3H); 4.39 (sext, J=6.3 Hz, 1H); 4.58 (d, J=6.3 Hz, 1H); 7.05-7.09 (m, 2H); 7.25 (d, J=8.4 Hz, 1H); 7.39 (t, J=7.9 Hz, 1H).

¹³C-NMR (CDCl₃, 100 MHz) δ: 21.3; 23.6; 26.2; 33.1; 52.8; 113.4; 126.9; 129.5; 129.8; 130.8; 132.9; 139.7; 156.4; 161.3; 166.9.

### N-cyclopentyl-2-methyl-5-(3-methylphenyl)pyrimidin-4-amine

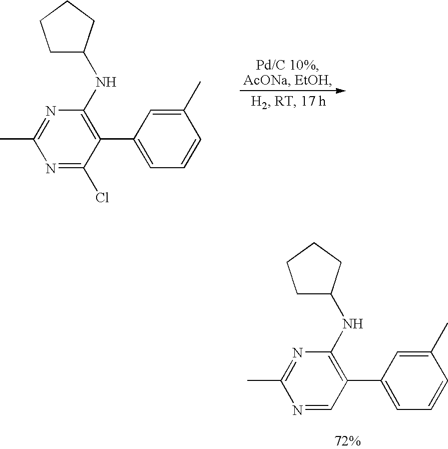

This compound was prepared according to the general procedure described for the example 12 starting from 50 mg (0.17 mmoles) of 6-chloro-N-cyclopentyl-2-methyl-5-(3-methylphenyl)pyrimidin-4-amine.

Aspect: colourless oil Mass obtained: 32 mg

Yield: 72%

LC-MS: Tᵣ=3.96 min. (100%) (ES-MS: m/z 268.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.34-1.45 (m, 2H); 1.57-1.70 (m, 4H); 1.96-2.05 (m, 2H); 2.38 (s, 3H); 2.47 (s, 3H); 4.46 (quint, J=7.9 Hz, 1H); 7.12-7.16 (m, 2H); 7.22 (d, J=7.9 Hz, 1H); 7.35 (t, J=7.9 Hz, 1H); 7.73 (s, 1H).

¹³C-NMR (CD₃OD, 75 MHz) δ: 20.3; 23.6; 24.6; 32.4; 52.4; 117.0; 125.6; 128.8; 129.2; 134.4; 139.3; 151.8; 159.8; 165.9.

### EXAMPLE 17

### 5-(3-butylphenyl)-6-chloro-N-cyclopentyl-2-methylpyrimidin-4-amine

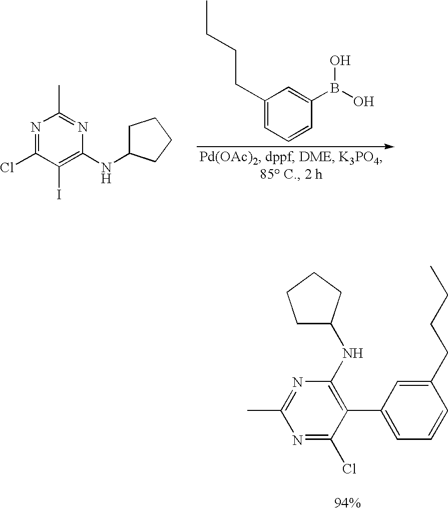

This compound was prepared according to the general procedure described for the example 12.

Aspect: yellow oil Mass obtained: 143 mg

Yield: 94%

LC-MS: Tᵣ=6.76 min. (93.8%) (ES-MS: m/z 344.2 (M); 346.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 0.93 (t, J=5.3 Hz, 3H); 1.24-1.29 (m, 2H); 1.35 (sext, J=6.3 Hz, 2H); 1.56-1.67 (m, 6H); 1.97-2.05 (m, 2H); 2.54 (s, 3H); 2.67 (t, J=6.3 Hz, 2H); 4.39 (sext, J=5.8 Hz, 1H); 4.59 (d, J=5.8 Hz, 1H); 7.06-7.09 (m, 2H); 7.24 (d, J=7.4 Hz, 1H); 7.39 (t, J=7.4 Hz, 1H).

¹³C-NMR (CDCl₃, 100 MHz) δ: 14.1; 22.3; 23.6; 26.2; 33.1; 33.8; 35.7; 52.8; 113.8; 127.2; 128.8; 129.5; 129.8; 132.8; 144.3; 156.1; 161.3; 166.6.

### 5-(3-butylphenyl)-N-cyclopentyl-2-methylpyrimidin-4-amine

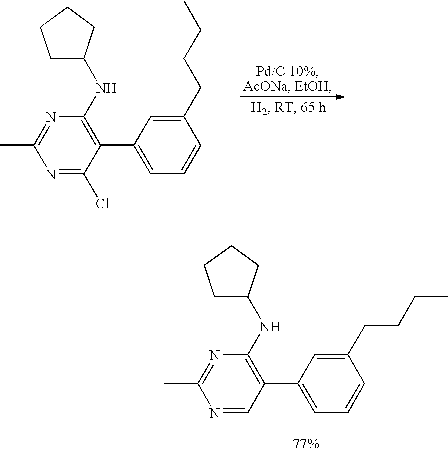

This compound was prepared according to the general procedure described for the example 12 starting from 45 mg (0.13 mmoles) of 5-(3-butylphenyl)-6-chloro-N-cyclopentyl-2-methylpyrimidin-4-amine.

Aspect: colourless oil Mass obtained: 31 mg

Yield: 77%

LC-MS: Tᵣ=4.87 min. (100%) (ES-MS: m/z 310.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 0.95 (t, J=7.1 Hz, 3H); 1.34-1.45 (m, 4H); 1.58-1.70 (m, 6H); 1.96-2.05 (m, 2H); 2.47 (s, 3H); 2.66 (t, J=7.9 Hz, 2H); 4.46 (quint, J=5.9 Hz, 1H); 7.14-7.17 (m, 2H); 7.24 (d, J=7.9 Hz, 1H); 7.38 (t, J=7.9 Hz, 1H); 7.76 (s, 1H).

¹³C-NMR (CD₃OD, 75 MHz) δ: 13.4; 22.3; 23.3; 24.3; 32.5; 33.8; 35.4; 52.4; 117.4; 125.9; 128.5; 128.6; 129.3; 134.1; 144.3; 151.8; 159.7; 165.9.

### EXAMPLE 18

### 5-(4-butylphenyl)-N,N′-dicyclopentyl-2-methylpyrimidine-4,6-diamine

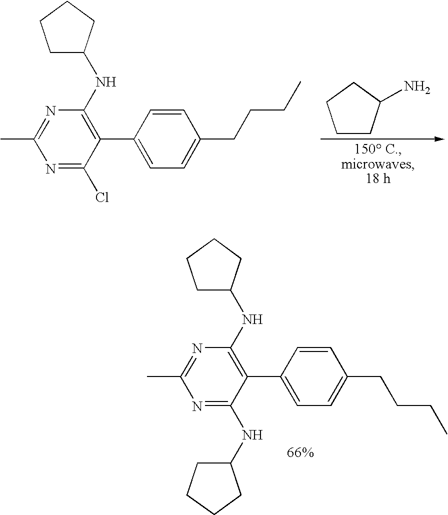

40 mg (0.12 mmoles, 1.0 eq.) of 5-(4-butylphenyl)-6-chloro-N-cyclopentyl-2-methylpyrimidin-4-amine were dissolved in 2 mL of cyclopentylamine and the solution was heated at 150° C. under microwaves irradiation for 18 h. Excess of reagent was removed under reduced pressure and the crude mixture was partitioned between 15 mL of AcOEt and 10 mL of water. The organic layer was washed once with 10 mL of water and once with 10 mL of brine. It was dried over Na₂SO₄, filtered and evaporated to dryness. The crude compound was purified by flash chromatography on silica gel to give 30 mg of a brown oil.

Yield: 66%

LC-MS: Tᵣ=5.92 min. (100%) (ES-MS: ink 393.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 0.97 (t, J=7.1 Hz, 3H); 1.17-1.30 (m, 4H); 1.41 (sext, J=7.9 Hz, 2H); 1.50-1.59 (m, 8H); 1.62-1.71 (m, 2H); 1.85-1.97 (m, 4H); 2.37 (s, 3H); 2.68 (t, J=7.9 Hz, 2H); 4.30 (quint, J=6.7 Hz, 2H); 7.10 (d, J=7.9 Hz, 2H); 7.35 (d, J=7.9 Hz, 2H).

¹³C-NMR (CD₃OD, 300 MHz) δ: 13.4; 21.0; 23.4; 24.7; 33.1; 33.8; 35.4; 52.4; 95.7; 130.2; 130.8; 143.3; 159.3; 165.6.

### EXAMPLE 19

### 5-(4-ethylphenyl)-N,N′-dicyclopentyl-2-methylpyrimidine-4,6-diamine

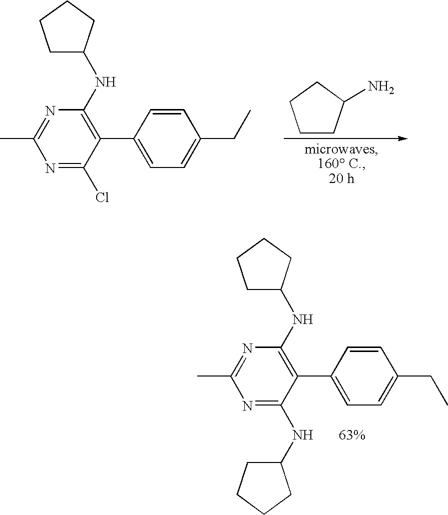

40 mg (0.13 mmoles, 1.0 eq.) of 6-chloro-N-cyclopentyl-5-(4-ethylphenyl)-2-methylpyrimidin-4-amine were dissolved in 2 mL of cyclopentylamine and the solution was heated at 160° C. for 20 h under microwaves irradiation. The excess of reagent was removed under reduced pressure and the black tarry residue was dissolved in 15 mL of AcOEt. The organic phase was washed twice with 10 mL of water and once with 10 mL of brine. It was dried over Na₂SO₄, filtered and evaporated to dryness. The crude residue was purified by flash chromatography on silica gel to give 29 mg of a brown oil.

Yield: 63%

LC-MS: Tᵣ=5.36 min. (100%) (ES-MS: m/z 365.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.16-1.31 (m, 7H); 1.51-1.60 (m, 8H); 1.85-1.96 (m, 4H); 2.37 (s, 3H); 2.71 (q, J=7.9 Hz, 2H); 4.30 (quint, J=6.3 Hz, 2H); 7.12 (d, J=7.9 Hz, 2H); 7.38 (d, J=7.9 Hz, 2H).

¹³C-NMR (CD₃OD, 75 MHz) δ: 15.1; 23.3; 24.6; 28.5; 33.1; 52.4; 95.7; 129.5; 130.2; 130.8; 144.6; 159.3; 165.6.

### EXAMPLE 20

### General procedure for the nucleophilic substitution of 6-chloropyrimidine by cyclopentylamine under microwaves irradiation

The corresponding 6-chloro-N-cyclopentyl-2-methyl-5-phenylpyrimidin-4-amine (1.0 eq.) was dissolved in 2 mL of cyclopentylamine. Two drops of BuMelm BF₄⁻ were added and the solution was heated at 200° C. under microwaves irradiation for 3 h. The excess of amine was removed by evaporation under reduced pressure and the crude compound was dissolved in 15 mL of AcOEt. The aqueous phase was washed twice with 10 mL of water and once with 10 mL of brine. It was dried over Na₂SO₄, filtered and evaporated to dryness. The crude compound was purified by flash chromatography on silica gel to give the desired derivative.

### 5-(4-methylphenyl)-N,N′-dicyclopentyl-2-methylpyrimidine-4,6-diamine

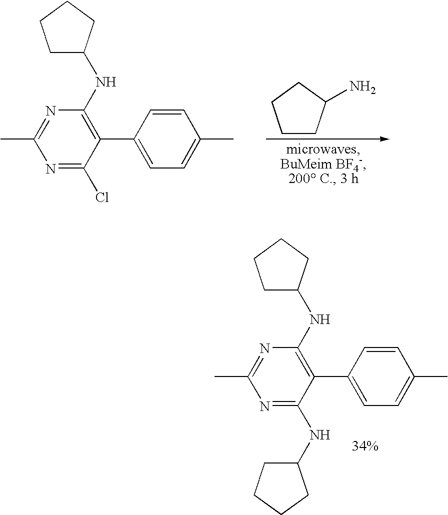

This compound was prepared according to the general procedure described above starting from 40 mg (0.13 mmoles) of 6-chloro-N-cyclopentyl-2-methyl-5-(4-methylphenyl)pyrimidin-4-amine.

Aspect: slightly brown solid Mass obtained: 15 mg

Yield: 34%

M.P.: 115-128° C.

LC-MS: Tᵣ=5.08 min. (100%) (ES-MS: m/z 351.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.17-1.29 (m, 4H); 1.51-1.60 (m, 8H); 1.85-1.96 (m, 4H); 2.37 (s, 3H); 2.41 (s, 3H); 4.30 (quint, J=7.1 Hz, 2H); 7.09 (d, J=7.9 Hz, 2H); 7.35 (d, J=7.9 Hz, 2H).

¹³C-NMR (CD₃OD, 75 MHz) δ: 20.3; 23.3; 24.6; 33.1; 52.8; 95.7; 129.8; 130.5; 130.8; 138.4; 159.3; 165.6.

### EXAMPLE 21

### 5-(4-methoxyphenyl)-N,N′-dicyclopentyl-2-methylpyrimidine-4,6-diamine

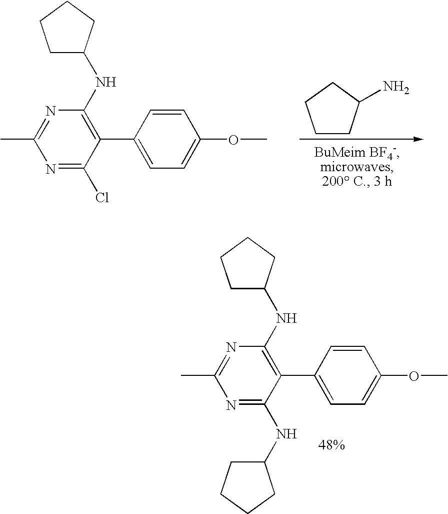

This compound was prepared according to the general procedure described for the example 20 starting from 40 mg (0.13 mmoles) of 6-chloro-N-cyclopentyl-5-(4-methoxyphenyl)-2-methylpyrimidin-4-amine.

Aspect: white solid Mass obtained: 22 mg

Yield: 48%

LC-MS: Tᵣ=4.90 min. (100%) (ES-MS: m/z 367.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.17-1.29 (m, 4H); 1.51-1.60 (m, 8H); 1.85-1.96 (m, 4H); 2.37 (s, 3H); 3.84 (s, 3H); 4.30 (quint., J=6.3 Hz, 2H); 7.06-7.13 (m, 4H).

¹³C-NMR (CD₃OD, 75 MHz) δ: 23.3; 24.6; 33.1; 52.8; 54.7; 95.4; 115.7; 124.3; 131.8; 159.7; 160.0; 165.2.

### EXAMPLE 22

### 5-(3-methylphenyl)-N,N′-dicyclopentyl-2-methylpyrimidine-4,6-diamine

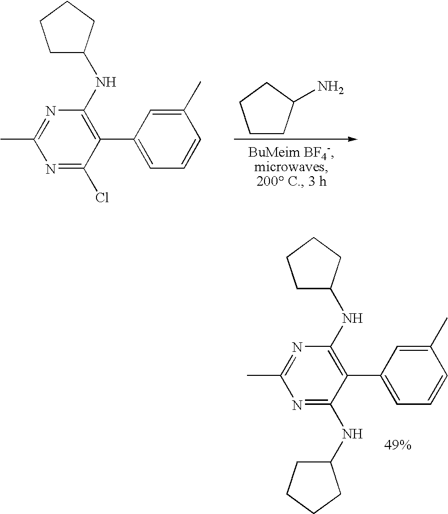

This compound was prepared according to the general procedure described for the example 20 starting from 40 mg (0.13 mmoles) of 6-chloro-N-cyclopentyl-2-methyl-5-(3-methylphenyl)pyrimidin-4-amine.

Aspect: white solid Mass obtained: 23 mg

Yield: 49%

M.P.: 95-98° C.

LC-MS: Tᵣ=5.07 min. (100%) (ES-MS: m/z 351.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.13-1.26 (m, 4H); 1.51-1.59 (m, 8H); 1.92-2.01 (m, 4H); 2.38 (s, 3H); 2.44 (s, 3H); 3.95 (d, J=7.9 Hz, 2H); 4.37 (sext, J=7.9 Hz, 2H); 7.00-7.03 (m, 2H); 7.17 (d, J=7.9 Hz, 1H); 7.35 (t, J=7.9 Hz, 1H).

### EXAMPLE 23

### 5-(3-butylphenyl)-N,N′-dicyclopentyl-2-methylpyrimidine-4,6-diamine

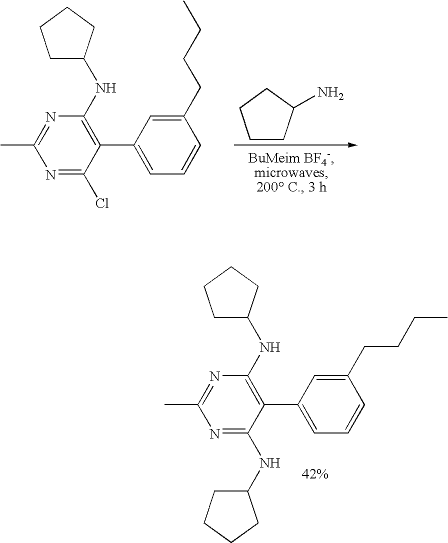

This compound was prepared according to the general procedure described for the example 20 starting from 40 mg (0.12 mmoles) of 5-(3-butylphenyl)-6-chloro-N-cyclopentyl-2-methylpyrimidin-4-amine.

Aspect: colourless oil Mass obtained: 19 mg

Yield: 42%

LC-MS: Tᵣ=5.88 min. (91%) (ES-MS: m/z 393.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 0.93 (t, J=6.3 Hz, 3H); 1.18-1.41 (m, 6H); 1.55-1.70 (m, 10H); 1.85-1.97 (m, 4H); 2.39 (s, 3H); 2.67 (t, J=7.9 Hz, 2H); 4.30 (quint, J=6.3 Hz, 2H); 7.00-8.04 (m, 2H); 7.26 (d, J=7.9 Hz, 1H); 7.44 (t, J=7.9 Hz, 1H).

¹³C-NMR (CD₃OD, 75 MHz) δ: 13.4; 22.3; 23.3; 24.6; 33.1; 33.8; 35.4; 52.4; 96.1; 127.9; 128.5; 130.2; 130.8; 133.1; 145.2; 159.3; 165.6.

### EXAMPLE 24

### 6-chloro-N-cyclopentyl-2-methyl-5-[4-(trifluoromethoxy)phenyl]pyrimidin-4-amine

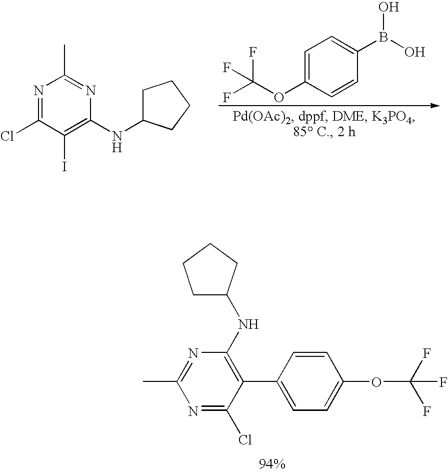

This compound was prepared according to the general procedure described for the example 12 starting from 300 mg (0.89 mmoles, 1.0 eq.) of 6-chloro-N-cyclopentyl-5-iodo-2-methylpyrimidin-4-amine.

Aspect: yellow oil Mass obtained: 312 mg

Yield: 94%

LC-MS: Tᵣ=6.23 min. (100%) (ES-MS: m/z 372.0 (M); 374.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.36-1.43 (m, 2H); 1.54-1.70 (m, 4H); 1.96-2.03 (m, 2H); 2.46 (s, 3H); 4.47 (quint, J=7.4 Hz, 1H); 7.38 (d, J=8.4 Hz, 2H); 7.43 (d, J=8.4 Hz, 2H).

¹³C-NMR (CD₃OD, 75 MHz) δ: 23.6; 24.6; 32.1; 53.1; 112.4; 120.7 (q, J=252.3 Hz); 121.6; 131.8; 132.1; 149.5; 155.7; 161.3; 166.9.

¹⁹F-NMR (CD₃OD, 282 MHz) δ: −59.9.

### N-cyclopentyl-2-methyl-5-[4-(trifluoromethoxy)phenyl]pyrimidin-4-amine

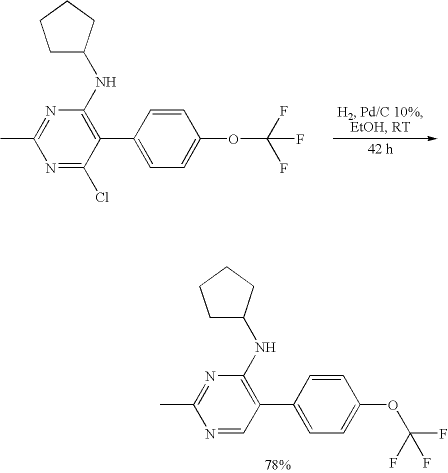

This compound was prepared according to the general procedure described for the example 12 starting from 100 mg (0.27 mmoles, 1.0 eq.) of 6-chloro-N-cyclopentyl-2-methyl-5-[4-(trifluoromethoxy)phenyl]pyrimidin-4-amine.

Aspect: white solid Mass obtained: 71 mg

Yield: 78%

M.P.: 46-54° C.

LC-MS: Tᵣ=4.35 min. (100%) (ES-MS: m/z 338.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.47 (m, 2H); 1.55-1.71 (m, 4H); 1.96-2.06 (m, 2H); 2.47 (s, 3H); 4.49 (quint, J=7.1 Hz, 1H); 7.38 (d, J=7.9 Hz, 2H); 7.47 (d, J=8.7 Hz, 2H); 7.78 (s, 1H).

¹³C-NMR (CD₃OD, 75 MHz) δ: 23.6; 24.3; 32.4; 52.4; 115.7; 120.6 (q, J=247.4 Hz); 122.0; 130.8; 133.8; 149.2; 152.4; 159.7; 166.6.

¹⁹F-NMR (CD₃OD, 282 MHz) δ: −60.2.

### EXAMPLE 25

### 6-chloro-N-cyclopentyl-2-methyl-5-[3-(trifluoromethoxy)phenyl]pyrimidin-4-amine

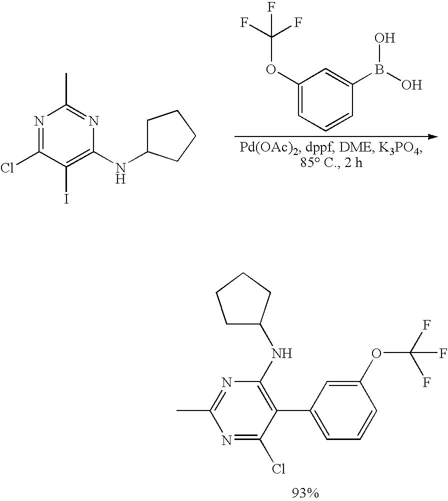

This compound was prepared according to the general procedure described for the example 12 starting from 300 mg (0.89 mmoles, 1.0 eq.) of 6-chloro-N-cyclopentyl-5-iodo-2-methylpyrimidin-4-amine.

Aspect: orange solid Mass obtained: 309 mg

Yield: 93%

M.P.: 46-53° C.

LC-MS: Tᵣ=6.33 min. (100%) (ES-MS: m/z 372.0 (M); 374.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.31-1.42 (m, 2H); 1.52-1.66 (m, 4H); 1.91-2.01 (m, 2H); 2.43 (s, 3H); 4.43 (quint, J=7.9 Hz, 1H); 7.20 (s, 1H); 7.26 (d, J=7.9 Hz, 1H); 7.34 (d, J=8.6 Hz, 1H); 7.59 (t, J=7.9 Hz, 1H).

¹³C-NMR (CD₃OD, 75 MHz) δ: 23.6; 24.6; 32.4; 53.1; 112.4; 120.6 (q, J=254.8 Hz); 121.3; 122.9; 129.2; 131.1; 135.1; 149.8; 155.7; 161.3; 166.9.

¹⁹F-NMR (CD₃OD, 282 MHz) δ: −60.0.

### N-cyclopentyl-2-methyl-5-[3-(trifluoromethoxy)phenyl]pyrimidin-4-amine

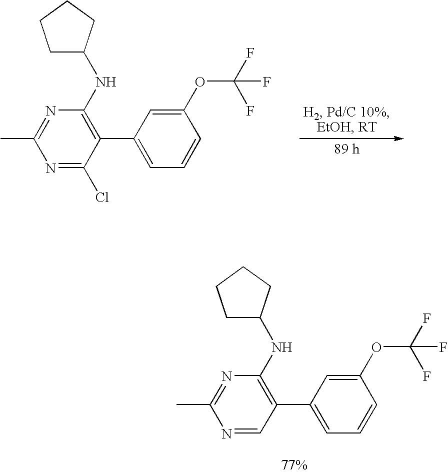

This compound was prepared according to the general procedure described for the example 12 starting from 100 mg (0.27 mmoles, 1.0 eq.) of 6-chloro-N-cyclopentyl-2-methyl-5-[3-(trifluoromethoxy)phenyl]pyrimidin-4-amine.

Aspect: white solid Mass obtained: 70 mg

Yield: 77%

M.P.: 90-94° C.

LC-MS: Tᵣ=4.30 min. (100%) (ES-MS: m/z 338.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.49 (m, 2H); 1.55-1.74 (m, 4H); 1.97-2.06 (m, 2H); 2.47 (s, 3H); 4.49 (quint, J=7.9 Hz, 1H); 7.29-7.33 (m, 2H); 7.38 (d, J=7.9 Hz, 1H); 7.58 (t, J=7.9 Hz, 1H); 7.79 (s, 1H).

¹³C-NMR (CD₃OD, 75 MHz) δ: 23.6; 24.6; 32.4; 52.8; 115.7; 120.6 (q, J=257.3 Hz); 120.6; 121.3; 127.5; 131.1; 137.0; 149.8; 152.4; 159.7; 166.6.

¹⁹F-NMR (CD₃OD, 282 MHz) δ: −60.2.

### EXAMPLE 26

### Ethyl 4-[4-chloro-6-(cyclopentylamino)-2-methylpyrimidin-5-yl]benzoate

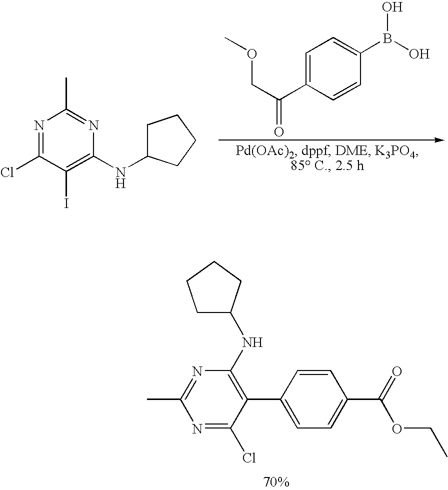

This compound was prepared according to the general procedure described for the example 12 starting from 300 mg (0.89 mmoles, 1.0 eq.) of 6-chloro-N-cyclopentyl-5-iodo-2-methylpyrimidin-4-amine.

Aspect: white solid Mass obtained: 225 mg

Yield: 70%

M.P.: 119-122° C.

LC-MS: Tᵣ=6.05 min. (100%) (ES-MS: m/z 360.2 (M); 362.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.35-1.45 (m, 5H); 1.56-1.68 (m, 4H); 1.96-2.04 (m, 2H); 2.49 (s, 3H); 4.39-4.51 (m, 3H); 7.42 (d, J=7.9 Hz, 2H); 8.16 (d, J=7.9 Hz, 2H).

¹³C-NMR (CD₃OD, 100 MHz) δ: 12.9; 23.2; 24.2; 31.9; 52.6; 61.0; 112.6; 130.0; 130.6; 137.4; 155.2; 161.0; 166.1; 166.8.

### Ethyl 4-[4-(cyclopentylamino)-2-methylpyrimidin-5-yl]benzoate

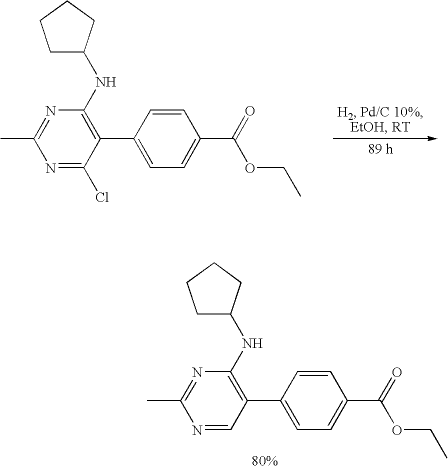

This compound was prepared according to the general procedure described for the example 12 starting from 100 mg (0.28 mmoles, 1.0 eq.) of Ethyl 4-[4-chloro-6-(cyclopentylamino)-2-methylpyrimidin-5-yl]benzoate.

Aspect: white solid Mass obtained: 72 mg

Yield: 80%

LC-MS: Tᵣ=4.00 min. (100%) (ES-MS: m/z 326.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.49 (m, 5H); 1.54-1.72 (m, 4H); 1.97-2.06 (m, 2H); 2.47 (s, 3H); 4.37 (q, J=7.9 Hz, 2H); 4.50 (quint, J=7.1 Hz, 1H); 7.49 (d, J=7.9 Hz, 2H); 7.81 (s, 1H); 8.09 (d, J=7.9 Hz, 2H).

¹³C-NMR (CD₃OD, 75 MHz) δ: 13.8; 23.6; 24.6; 32.4; 52.4; 61.3; 116.1; 128.9; 130.2; 130.5; 139.7; 152.1; 159.3; 166.2; 166.5.

### EXAMPLE 27

### 4-[4-(cyclopentylamino)-2-methylpyrimidin-5-yl]benzoic acid

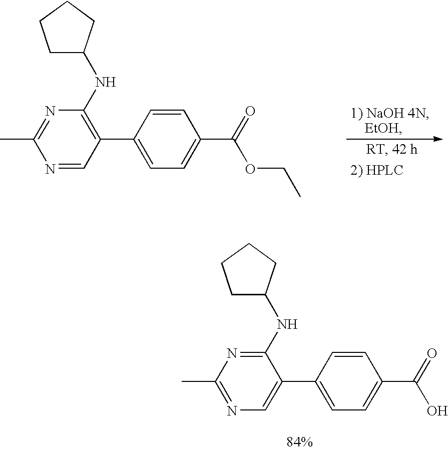

To a solution of 30 mg (0.09 mmoles, 1.0 eq.) of Ethyl 4-[4-(cyclopentylamino)-2-methylpyrimidin-5-yl]benzoate in 70 μL of EtOH was added 35 μL of NaOH 4N. The mixture was stirred at RT for 42 h. Solvents were removed under reduced pressure and the crude compound was purified by preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 mL/min.).

Aspect: white solid Mass obtained: 23 mg

Yield: 84%

M.P.: 252-255° C.

LC-MS: Tᵣ=3.16 min. (100%) (ES-MS: m/z 298.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.47-1.55 (m, 2H); 1.62-1.79 (m, 4H); 2.01-2.10 (m, 2H); 2.58 (s, 3H); 4.62 (quint., J=5.5 Hz, 1H); 7.53 (d, J=8.4 Hz, 2H); 7.92 (s, 1H); 8.16 (d, J=8.4 Hz, 2H).

### EXAMPLE 28

### 6-chloro-N-cyclopentyl-2-methyl-5-[4-(trifluoromethyl)phenyl]pyrimidin-4-amine

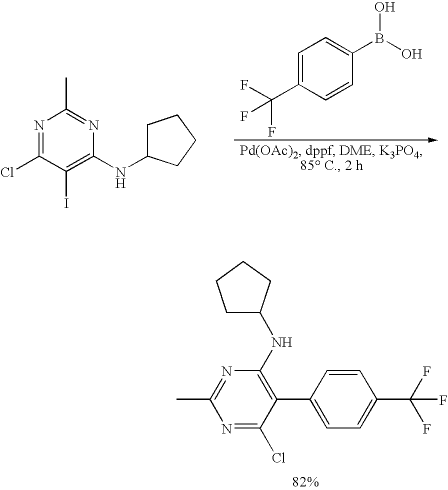

This compound was prepared according to the general procedure described for the example 12 starting from 200 mg (0.59 mmoles, 1.0 eq.) of 6-chloro-N-cyclopentyl-5-iodo-2-methylpyrimidin-4-amine.

Aspect: white solid Mass obtained: 174 mg

Yield: 82%

M.P.: 135-138° C.

LC-MS: Tᵣ=6.25 min. (100%) (ES-MS: m/z 356.2 (M); 358.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.17-1.29 (m, 2H); 1.50-1.63 (m, 4H); 1.93-2.05 (m, 2H); 2.50 (s, 3H); 4.31-4.47 (m, 2H); 7.38 (d, J=7.9 Hz, 2H); 7.71 (d, J=7.9 Hz, 2H).

¹³C-NMR (CDCl₃, 75 MHz) δ: 23.9; 26.2; 33.4; 53.4; 112.1; 123.9 (q, J=272.1 Hz); 127.2; 130.8; 131.1 (q, J=24.7 Hz); 137.0; 156.4; 160.7; 167.5.

¹⁹F-NMR (CDCl₃, 282 MHz) δ: −63.5.

### N-cyclopentyl-2-methyl-5-[4-(trifluoromethyl)phenyl]pyrimidin-4-amine

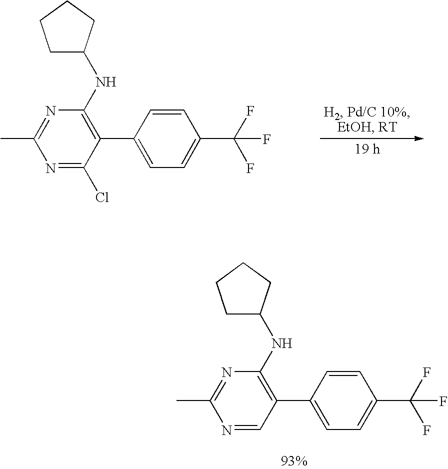

This compound was prepared according to the general procedure described for the example 12 starting from 80 mg (0.23 mmoles, 1.0 eq.) of 6-chloro-N-cyclopentyl-2-methyl-5-[4-(trifluoromethyl)phenyl]pyrimidin-4-amine.

Aspect: white solid Mass obtained: 67 mg

Yield: 93%

M.P.: 94-97° C.

LC-MS: Tᵣ=4.19 min. (100%) (ES-MS: m/z 322.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.38-1.49 (m, 2H); 1.56-1.71 (m, 4H); 1.96-2.06 (m, 2H); 2.47 (s, 3H); 4.50 (quint, J=7.9 Hz, 1H); 7.57 (d, J=7.9 Hz, 2H); 7.78 (d, J=7.9 Hz, 2H); 7.82 (s, 1H).

¹⁹F-NMR (CDCl₃, 282 MHz) δ: −63.5.

### EXAMPLE 29

### 6-chloro-N-cyclopentyl-2-methyl-5-[3-(trifluoromethyl)phenyl]pyrimidin-4-amine

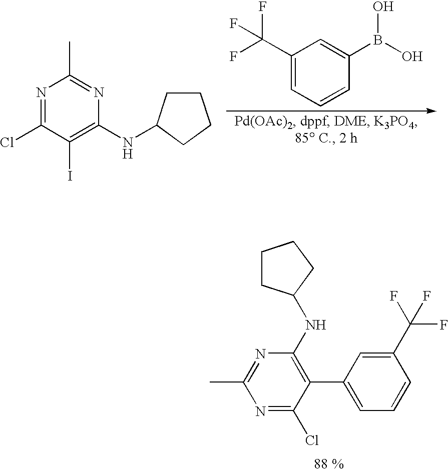

This compound was prepared according to the general procedure described for the example 12 starting from 200 mg (0.59 mmoles, 1.0 eq.) of 6-chloro-N-cyclopentyl-5-iodo-2-methylpyrimidin-4-amine.

Aspect: slightly yellow solid Mass obtained: 186 mg

Yield: 88%

M.P.: 97-100° C.

LC-MS: Tᵣ=6.17 min. (100%) (ES-MS: m/z 356.0 (M); 358.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.31-1.42 (m, 2H); 1.50-1.68 (m, 4H); 1.92-2.00 (m, 2H); 2.46 (s, 3H); 4.46 (quint, J=7.9 Hz, 1H); 7.52 (d, J=7.9 Hz, 1H); 7.58 (s, 1H); 7.66-7.78 (m, 2H).

¹³C-NMR (CD₃OD, 75.45 MHz) δ: 23.6; 24.6; 32.1; 53.1; 112.4; 124.3 (q, J=262.2 Hz); 125.2; 126.9; 130.2; 131.5 (q, J=39.6 Hz); 133.8; 155.7; 161.3; 166.9.

¹⁹F-NMR (CD₃OD, 282 MHz) δ: −64.8.

### N-cyclopentyl-2-methyl-5-[3-(trifluoromethyl)phenyl]pyrimidin-4-amine

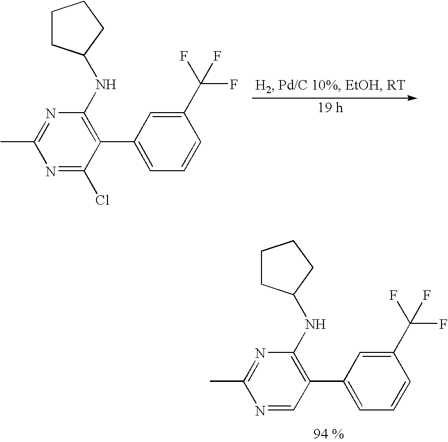

This compound was prepared according to the general procedure described for the example 12 starting from 80 mg (0.23 mmoles, 1.0 eq.) of 6-chloro-N-cyclopentyl-2-methyl-5-[3-(trifluoromethyl)phenyl]pyrimidin-4-amine.

Aspect: white solid Mass obtained: 68 mg

Yield: 94%

M.P.: 105-108° C.

LC-MS: Tᵣ=4.18 min. (100%) (ES-MS: m/z 322.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.21-1.38 (m, 2H); 1.55-1.68 (m, 4H); 2.00-2.10 (m, 2H); 2.54 (s, 3H); 4.45 (sext, J=7.1 Hz, 1H); 4.72 (d, J=7.1 Hz, 1H); 7.51-7.66 (m, 4H); 7.89 (s, 1H).

¹⁹F-NMR (CDCl₃, 282 MHz) δ: −63.7.

### EXAMPLE 30

### 5-[3,5-bis(trifluoromethyl)phenyl]-6-chloro-N-cyclopentyl-2-methylpyrimidin-4-amine

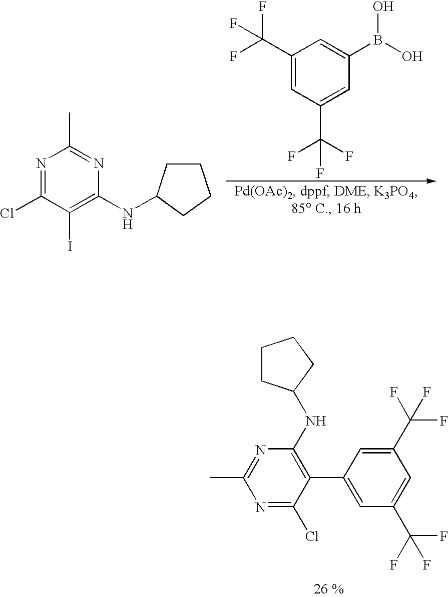

This compound was prepared according to the general procedure described for the example 12 starting from 200 mg (0.59 mmoles, 1.0 eq.) of 6-chloro-N-cyclopentyl-5-iodo-2-methylpyrimidin-4-amine.

Aspect: white solid Mass obtained: 66 mg

Yield: 26%

M.P.: 108-118° C.

LC-MS: Tᵣ=3.97 min. (100%) (ES-MS: m/z 424.0 (M); 426.0 (M+2)) [Column: Nucleosil C-18HD, 4×70 mm, 3 μm, gradient CH₃CN/H₂O/TFA 0.05%: 65-100% CH₃CN (6 min.), 100% CH₃CN (1.5 min.), flow: 1 mL/min].

¹H-NMR (CDCl₃, 300 MHz) δ: 1.21-1.34 (m, 2H); 1.55-1.66 (m, 4H); 1.99-2.09 (m, 2H); 2.54 (s, 3H); 4.29-4.46 (m, 2H); 7.76 (s, 2H); 7.92 (s, 1H).

¹⁹F-NMR (CDCl₃, 282 MHz) δ: −63.9.

### 5-[3,5-bis(trifluoromethyl)phenyl]-N-cyclopentyl-2-methylpyrimidin-4-amine

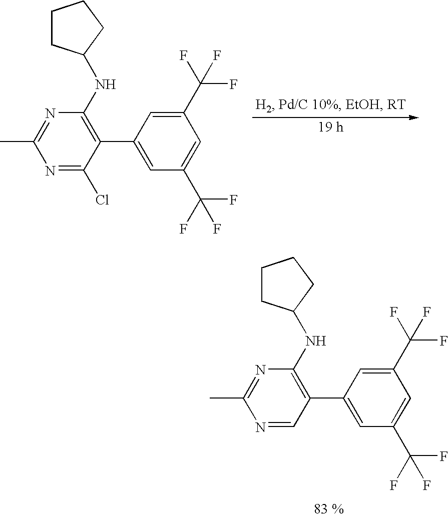

This compound was prepared according to the general procedure described for the example 12 starting from 80 mg (0.23 mmoles, 1.0 eq.) of 5-[3,5-bis(trifluoromethyl)phenyl]-6-chloro-N-cyclopentyl-2-methylpyrimidin-4-amine.

Aspect: white solid Mass obtained: 38 mg

Yield: 83%

M.P.: 124-126° C.

LC-MS: Tᵣ=4.57 min. (100%) (ES-MS: m/z 390.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.39-1.50 (m, 2H); 1.56-1.72 (m, 4H); 1.96-2.05 (m, 2H); 2.48 (s, 3H); 4.53 (quint, J=7.1 Hz, 1H); 7.85 (s, 1H); 7.96-7.99 (m, 3H).

¹³C-NMR (CD₃OD, 75 MHz) δ: 23.6; 24.6; 32.1; 52.8; 114.4; 121.6; 123.3 (q, J=270.5 Hz); 129.5; 132.1 (q, J=34.6 Hz); 137.7; 152.8; 159.7; 167.2.

¹⁹F-NMR (CD₃OD, 282 MHz) δ: −65.2.

### EXAMPLE 31

### 6-chloro-N-cyclopentyl-5-(3,4-dimethoxyphenyl)-2-methylpyrimidin-4-amine

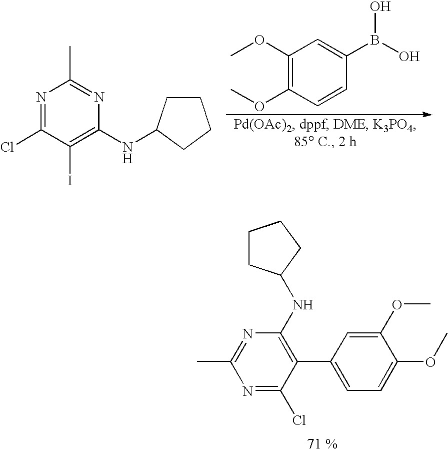

This compound was prepared according to the general procedure described for the example 12 starting from 200 mg (0.59 mmoles, 1.0 eq.) of 6-chloro-N-cyclopentyl-5-iodo-2-methylpyrimidin-4-amine

Aspect: white foam Mass obtained: 147 mg

Yield: 71%

LC-MS: Tᵣ=4.81 min. (100%) (ES-MS: m/z 348.2 (M); 350.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.20-1.29 (m, 2H); 1.53-1.62 (m, 4H); 1.93-2.04 (m, 2H); 2.51 (s, 3H); 3.85 (s, 3H); 3.92 (s, 3H); 4.35 (sext, J=7.9 Hz, 1H); 4.60 (d, J=7.9 Hz, 1H); 6.74 (d, J=1.6 Hz, 1H); 6.79 (dd, J=7.9 Hz, J=1.6 Hz, 1H); 6.96 (d, J=7.9 Hz, 1H).

### N-cyclopentyl-5-(3,4-dimethoxyphenyl)-2-methylpyrimidin-4-amine

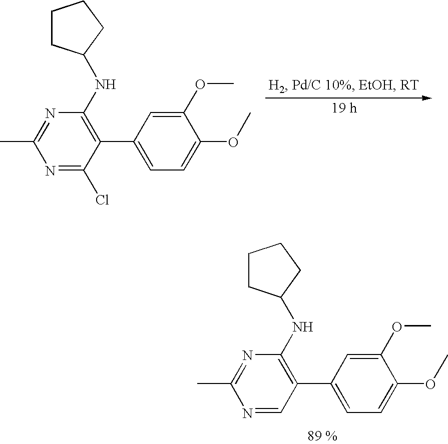

This compound was prepared according to the general procedure described for the example 12 starting from 80 mg (0.23 mmoles, 1.0 eq.) of 6-chloro-N-cyclopentyl-5-(3,4-dimethoxyphenyl)-2-methylpyrimidin-4-amine.

Aspect: white solid Mass obtained: 64 mg

M.P.: 143-145° C.

LC-MS: Tᵣ=4.81 min. (100%) (ES-MS: m/z 314.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.47 (m, 2H); 1.58-1.74 (m, 4H); 1.97-2.06 (m, 2H); 2.46 (s, 3H); 3.83 (s, 3H); 3.87 (s, 3H); 4.47 (quint, J=7.1 Hz, 1H); 6.89-6.92 (m, 2H); 7.05 (d, J=7.9 Hz, 1H); 7.76 (s, 1H).

¹³C-NMR (CD₃OD, 75 MHz) δ: 23.6; 24.3; 32.4; 52.4; 55.4; 112.1; 112.4; 117.0; 121.3; 126.9; 149.5; 150.0; 151.5; 159.7; 165.9.

### EXAMPLE 32

### 4-[4-chloro-6-(cyclopentylamino)-2-methylpyrimidin-5-yl]benzamide

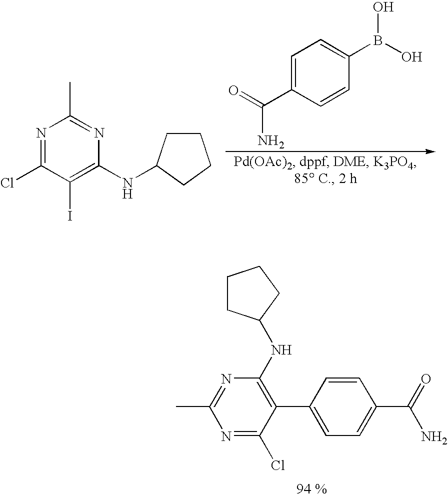

This compound was prepared according to the general procedure described for the example 12 starting from 200 mg (0.59 mmoles, 1.0 eq.) of 6-chloro-N-cyclopentyl-5-iodo-2-methylpyrimidin-4-amine

Aspect: yellow solid Mass obtained: 185 mg

Yield: 94%

M.P.: 209-212° C.

LC-MS: Tᵣ=3.89 min. (100%) (ES-MS: m/z 331.2 (M+H); 333.2 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.13-1.24 (m, 2H); 1.46-1.58 (m, 4H); 1.89-2.00 (m, 2H); 2.46 (s, 3H); 4.33 (sext, J=7.1 z, 1H); 4.46 (d, J=7.9 Hz, 1H); 6.74 (s, 2H); 7.31 (d, J=7.9 Hz, 2H); 7.92 (d, J=7.9 Hz, 2H).

¹³C-NMR (CDCl₃, 75 MHz) δ: 23.9; 26.2; 33.4; 53.1; 112.4; 128.8; 130.6; 134.1; 136.7; 156.4; 161.0; 167.3; 169.5.

### 4-[4-(cyclopentylamino)-2-methylpyrimidin-5-yl]benzamide

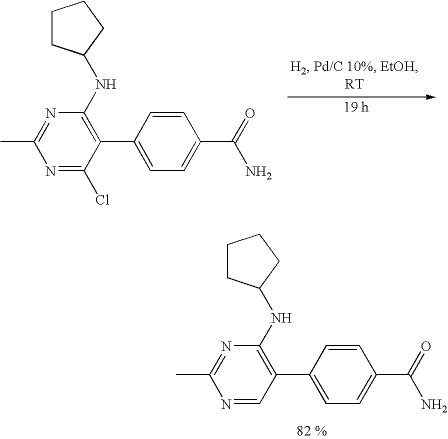

This compound was prepared according to the general procedure described for the example 12 starting from 80 mg (0.23 mmoles, 1.0 eq.) of 4-[4-chloro-6-(cyclopentylamino)-2-methylpyrimidin-5-yl]benzamide.

Aspect: white solid Mass obtained: 59 mg

Yield: 82%

M.P.: 226-228° C.

LC-MS: Tᵣ=2.68 min. (100%) (ES-MS: m/z 297.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.47 (m, 2H); 1.55-1.74 (m, 4H); 1.97-2.06 (m, 2H); 2.49 (s, 3H); 4.50 (quint, J=7.1 Hz, 1H); 7.49 (d, J=7.9 Hz, 2H); 7.80 (s, 1H); 7.97 (d, J=7.9 Hz, 2H).

¹³C-NMR (CD₃OD, 75 MHz) δ: 23.6; 24.6; 32.5; 52.4; 116.1; 128.5; 128.9; 133.4; 138.4; 152.1; 159.3; 166.6; 170.5.

### EXAMPLE 33

### N-cyclopentyl-2-methylpyrimidin-4-amine

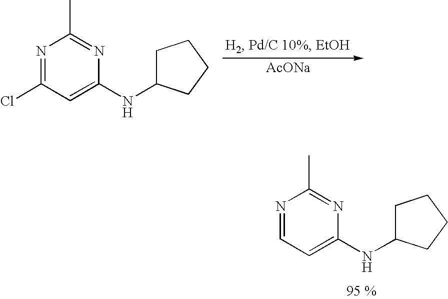

This compound was prepared according to the general procedure described for the example 12 starting from 3.81 g (18.0 mmoles, 1.0 eq.) of 6-chloro-N-cyclopentyl-2-methylpyrimidin-4-amine.

Aspect: colourless oil Mass obtained: 3.05 g

Yield: 95%

LC-MS: Tᵣ=1.95 min. (100%) (ES-MS: m/z 178.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.43-1.53 (m, 2H); 1.58-1.79 (m, 4H); 1.96-2.06 (m, 2H); 2.45 (s, 3H); 3.92 (m, 1H); 6.13 (d, J=6.3 Hz, 1H); 8.08 (d, J=6.3 Hz, 1H).

### 5-bromo-N-cyclopentyl-2-methylpyrimidin-4-amine

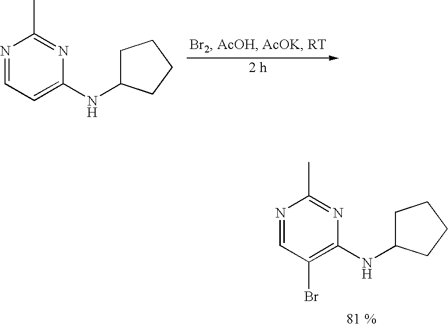

3.05 g (17.21 mmoles, 1.0 eq.) of N-cyclopentyl-2-methylpyrimidin-4-amine and 2.20 g (22.37 mmoles, 1.3 eq.) of AcOK were dissolved in 69.2 mL of AcOH. The solution was cooled to 0° C. and a solution of 1.06 mL (20.65 mmoles, 1.2 eq.) of bromine in 4.77 mL of AcOH was added slowly over 4 min. The mixture was allowed to reach RT and was stirred for 2 h. The crude mixture was poured into iced water. The aqueous phase was alcalinized with NaOH 2N and extracted four times with 100 mL of AcOEt. The combined organic layers were washed once with 100 mL of a saturated Na₂CO₃ solution and once with 100 mL of brine. The organic layer was dried over Na₂SO₄, filtered and evaporated to dryness. The crude compound was purified by flash chromatography on silica gel to give 3.58 g of a yellow solid.

Yield: 81%

M.P.: 78-83° C.

LC-MS: Tᵣ=2.63 min. (100%) (ES-MS: m/z 256.0 (M); 258.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.39-1.50 (m, 2H); 1.59-1.80 (m, 4H); 2.05-2.16 (m, 2H); 2.47 (s, 3H); 4.42 (sext, J=7.1 Hz, 1H); 5.18 (m, 1H); 8.12 (s, 1H).

### N-cyclopentyl-2-methyl-5-(4-nitrophenyl)pyrimidin-4-amine

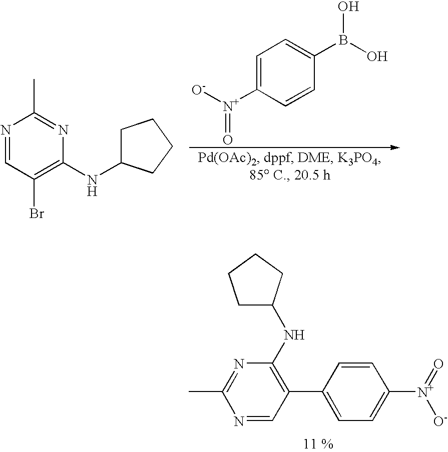

This compound was prepared according to the general procedure described for the example 12 starting from 120 mg (0.47 mmoles, 1.0 eq.) of 5-bromo-N-cyclopentyl-2-methylpyrimidin-4-amine.

Aspect: yellow solid Mass obtained: 15 mg

Yield: 11%

M.P.: 185-188° C.

LC-MS: Tᵣ=3.65 min. (100%) (ES-MS: m/z 299.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.22-1.39 (m, 2H); 1.58-1.71 (m, 4H); 2.03-2.14 (m, 2H); 2.56 (s, 3H); 4.47 (quint, J=7.1 Hz, 1H); 7.55 (d, J=7.9 Hz, 2H); 7.95 (s, 1H); 8.31 (d, J=7.9 Hz, 2H).

### EXAMPLE 34

### 5-(1,3-benzodioxol-5-yl)-N-cyclopentyl-2-methylpyrimidin-4-amine

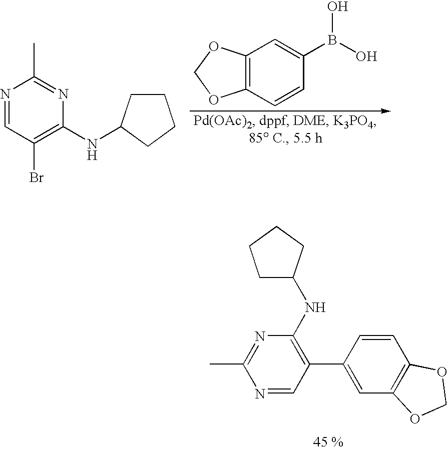

This compound was prepared according to the general procedure described for the example 12 starting from 120 mg (0.47 mmoles, 1.0 eq.) of 5-bromo-N-cyclopentyl-2-methylpyrimidin-4-amine.

Aspect: yellow oil Mass obtained: 63 mg

Yield: 45%

LC-MS: Tᵣ=3.65 min. (100%) (ES-MS: m/z 298.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 30 MHz) δ: 1.34-1.46 (m, 2H); 1.54-1.71 (m, 4H); 1.96-2.05 (m, 2H); 2.45 (s, 3H); 4.45 (quint, J=7.1 Hz, 1H); 5.99 (s, 2H); 6.78-6.80 (m, 2H); 6.89 (d, J=7.9 Hz, 1H); 7.72 (s, 1H).

¹³C-NMR (CD₃OD, 75 MHz) δ: 23.6; 24.3; 32.4; 52.4; 101.6; 108.8; 109.2; 117.0; 122.3; 127.5; 147.9; 148.8; 151.5; 159.7; 165.9.

### EXAMPLE 35

### N-cyclopentyl-2-methyl-5-(3-nitrophenyl)pyrimidin-4-amine

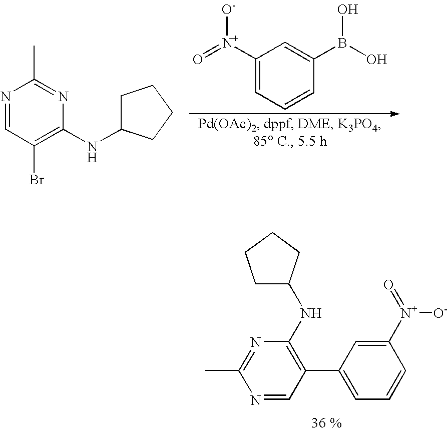

This compound was prepared according to the general procedure described for the example 12 starting from 120 mg (0.47 mmoles, 1.0 eq.) of 5-bromo-N-cyclopentyl-2-methylpyrimidin-4-amine.

Aspect: yellow oil Mass obtained: 51 mg

Yield: 36%

LC-MS: Tᵣ=3.64 min. (100%) (ES-MS: m/z 299.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.27-1.39 (m, 2H); 1.55-1.70 (m, 4H); 2.01-2.13 (m, 2H); 2.54 (s, 3H); 4.46 (sext, J=7.1 Hz, 1H); 4.74 (d, J=7.1 Hz, 1H); 7.62-7.71 (m, 2H); 7.92 (s, 1H); 8.20-8.24 (m, 2H).

### EXAMPLE 36

### 4-[4-(cyclopentylamino)-2-methylpyrimidin-5-yl]benzonitrile

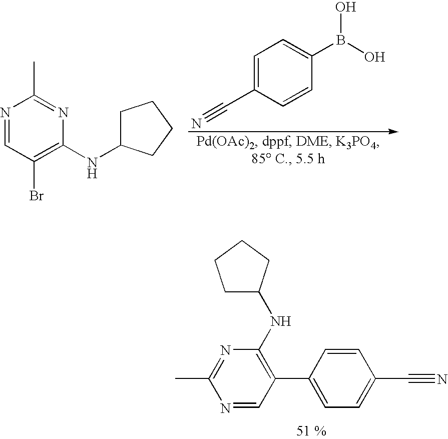

This compound was prepared according to the general procedure described for the example 12 starting from 120 mg (0.47 mmoles, 1.0 eq.) of 5-bromo-N-cyclopentyl-2-methylpyrimidin-4-amine.

Aspect: white solid Mass obtained: 66 mg

Yield: 51%

M.P.: 176-180° C.

LC-MS: Tᵣ=3.44 min. (100%) (ES-MS: m/z 279.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.39-1.50 (m, 2H); 1.55-1.74 (m, 4H); 1.97-2.08 (m, 2H); 2.47 (s, 3H); 4.51 (quint, J=7.1 Hz, 1H); 7.58 (d, J=7.9 Hz, 2H); 7.80-7.84 (m, 3H).

### EXAMPLE 37

### N-cyclopentyl-5-(3,4-difluorophenyl)-2-methylpyrimidin-4-amine

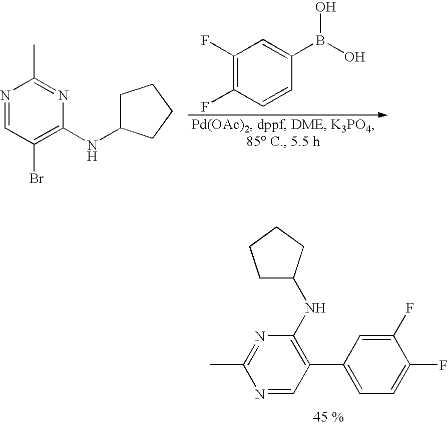

This compound was prepared according to the general procedure described for the example 12 starting from 120 mg (0.47 mmoles, 1.0 eq.) of 5-bromo-N-cyclopentyl-2-methylpyrimidin-4-amine.

Aspect: white solid Mass obtained: 61 mg

Yield: 45%

LC-MS: Tᵣ=3.77 min. (100%) (ES-MS: m/z 290.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.38-1.49 (m, 2H); 1.54-1.72 (m, 4H); 1.96-2.06 (m, 2H); 2.46 (s, 3H); 4.49 (quint, J=7.1 Hz, 1H); 7.13-7.18 (m, 1H); 7.26-7.39 (m, 2H); 7.76 (s, 1H).

¹³C-NMR (CD₃OD, 75 MHz) δ: 23.6; 24.6; 32.4; 52.8; 115.1; 118.0-118.4 (m); 125.6; 131.8; 148.8 (dd, J=29.7 Hz, J=9.9 Hz); 152.1-152.5 (m); 159.7; 166.6.

¹⁹F-NMR (CDCl₃, 282 MHz) δ: −137.6; −135.7.

### EXAMPLE 38

### General Procedure for the Suzuki Cross-Coupling Reaction Using Pd(PPh₃)₄

A solution of 108 mg (1.00 mmoles, 2.55 eq.) of sodium carbonate in 784 μL of water was added to a mixture of 100 mg (0.39 mmoles, 1.0 eq.) of 5-bromo-N-cyclopentyl-2-methylpyrimidin-4-amine, 0.43 mmoles (1.10 eq.) of the corresponding benzeneboronic acid and 16 mg (0.014 mmoles, 0.04 eq.) of Pd(PPh₃)₄ in 784 μL of toluene and 784 μL of EtOH. The mixture was heated at 110° C. for 3 h under argon. The mixture was allowed to cool to RT and 15 mL of AcOEt were added followed by 10 mL of water. The aqueous phase was extracted twice with 15 mL of AcOEt. The combined organic layers were washed once with 10 mL of brine, dried over Na₂SO₄, filtered and evaporated to dryness. The crude compound was purified by flash chromatography on silica gel to give the desired compound.

### N-cyclopentyl-2-methyl-5-phenylpyrimidin-4-amine

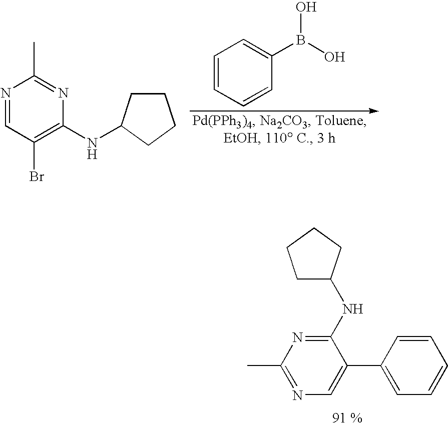

This compound was prepared according to the general procedure described above.

Aspect: yellow oil Mass obtained: 90 mg

Yield: 91%

LC-MS: Tᵣ=3.57 min. (100%) (ES-MS: m/z 254.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.33-1.45 (m, 2H); 1.54-1.71 (m, 4H); 1.95-2.05 (m, 2H); 2.46 (s, 3H); 4.46 (quint, J=7.1 Hz, 1H); 7.33-7.50 (m, 5H); 7.76 (m, 1H).

¹³C-NMR (CD₃OD, 75 MHz) δ: 23.6; 24.6; 32.4; 52.4; 117.0; 128.2; 128.5; 129.5; 134.4; 151.8; 159.7; 166.2.

### EXAMPLE 39

### N-cyclopentyl-5-(4-fluorophenyl)-2-methylpyrimidin-4-amine

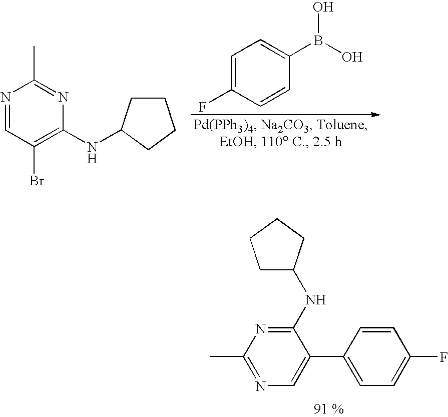

This compound was prepared according to the general procedure described for the example 38.

Aspect: yellow solid Mass obtained: 97 mg

Yield: 91%

M.P.: 83-86° C.

LC-MS: Tᵣ=3.68 min. (100%) (ES-MS: m/z 272.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.35-1.46 (m, 2H); 1.54-1.71 (m, 4H); 1.96-2.05 (m, 2H); 2.46 (s, 3H); 4.47 (quint, J=7.1 Hz, 1H); 7.18-7.24 (m, 2H); 7.34-7.39 (m, 2H); 7.75 (s, 1H).

¹³C-NMR (CD₃OD, 75 MHz) δ: 23.6; 24.3; 32.4; 52.4; 116.1 (d, J=24.7 Hz); 130.5; 130.8 (d, J=5 Hz); 152.1; 159.7; 161.3; 164.6; 166.2.

¹⁹F-NMR (CD₃OD, 282 MHz) δ: −115.7.

### EXAMPLE 40

### N-cyclopentyl-2-methyl-6-[4-(methylsulfonyl)phenyl]pyrimidin-4-amine

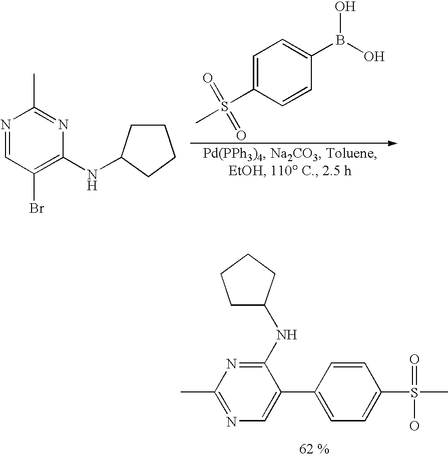

This compound was prepared according to the general procedure described for the example 38.

Aspect: white solid Mass obtained: 80 mg

Yield: 6270

M.P.: 160-164° C.

LC-MS: Tᵣ=3.14 min. (100%) (ES-MS: m/z 332.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.39-1.50 (m, 2H); 1.54-1.75 (m, 4H); 1.97-2.06 (m, 2H); 2.47 (s, 3H); 3.15 (s, 3H); 4.51 (quint, J=7.1 Hz, 1H); 7.66 (d, J=7.9 Hz, 2H); 7.83 (s, 1H); 8.03 (d, J=7.9 Hz, 2H).

¹³C-NMR (CD₃OD, 75 MHz) δ: 23.89; 24.6; 32.4; 43.3; 52.4; 115.4; 128.2; 129.8; 140.3; 140.6; 152.8; 159.3; 166.9.

### EXAMPLE 41

### 5-[2,4-bis(trifluoromethyl)phenyl]-N-cyclopentyl-2-methylpyrimidin-4-amine

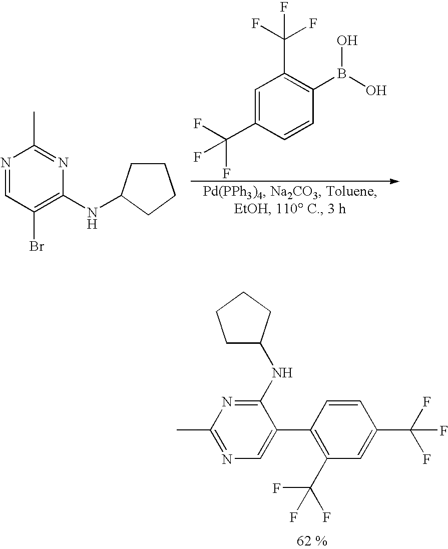

This compound was prepared according to the general procedure described for the example 38.

Aspect: white solid Mass obtained: 94 mg

Yield: 62%

M.P.: 128-132° C.

LC-MS: Tᵣ=4.45 min. (100%) (ES-MS: m/z 390.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.34-1.46 (m, 2H); 1.53-1.70 (m, 4H); 1.89-2.01 (m, 2H); 2.50 (s, 3H); 4.54 (quint, J=7.1 Hz, 1H); 7.56 (d, J=7.9 Hz, 1H); 7.67 (s, 1H); 8.01 (d, J=7.9 Hz, 1H); 8.09 (s, 1H).

¹³C-NMR (CD₃OD, 75 MHz) δ: 23.9; 24.6; 31.8; 52.4; 113.1; 123.4 (q, J=272.1 Hz); 123.6 (q, J=272.1 Hz); 123.6; 129.5; 130.5-132.1 (2 overlapping quadruplet); 134.4; 137.7; 152.4; 159.7; 167.2.

¹⁹F-NMR (CD₃OD, 282 MHz) δ: −62.0; −65.2.

### EXAMPLE 42

### 5-(4-chlorophenyl)-N-cyclopentyl-2-methylpyrimidin-4-amine

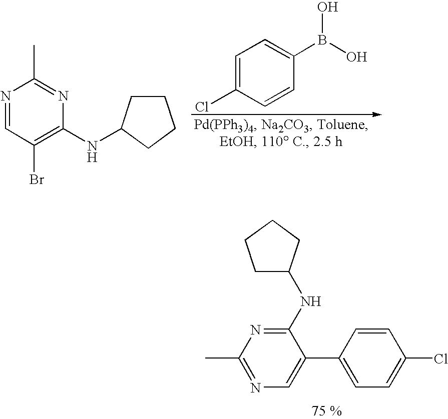

This compound was prepared according to the general procedure described for the example 38.

Aspect: white solid Mass obtained: 84 mg

Yield: 75%

M.P.: 103-106° C.

LC-MS: Tᵣ=4.00 min. (100%) (ES-MS: m/z 288.2 (M); 290.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.37-1.47 (m, 2H); 1.54-1.74 (m, 4H); 1.96-2.05 (m, 2H); 2.46 (s, 3H); 4.47 (quint, J=7.1 Hz, 1H); 7.34 (d, J=7.9 Hz, 2H); 7.47 (d, J=7.9 Hz, 2H); 7.76 (s, 1H).

¹³C-NMR (CD₃OD, 75.45 MHz) δ: 23.5; 24.5; 32.2; 52.2; 115.8; 129.3; 130.3; 133.2; 134.2; 151.9; 159.3; 166.4.

### EXAMPLE 43

### N-cyclopentyl-5-(2,4-dichlorophenyl)-2-methylpyrimidin-4-amine

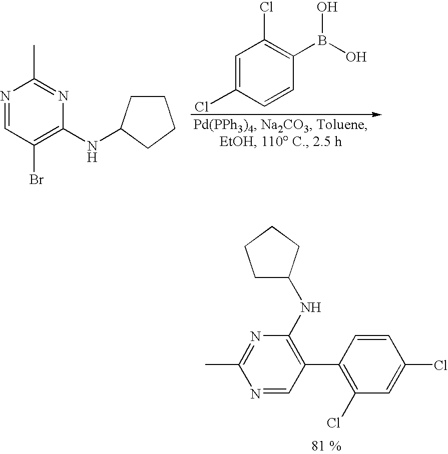

This compound was prepared according to the general procedure described for the example 38.

Aspect: white solid Mass obtained: 102 mg

Yield: 81%

M.P.: 111-116° C.

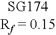

LC-MS: Tᵣ=4.19 min. (100%) (ES-MS: m/z 322.0 (M); 324.0 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.21-1.42 (m, 2H); 1.54-1.67 (m, 4H); 1.96-2.10 (m, 2H); 2.55 (s, 3H); 4.29 (d, J=7.1 Hz, 1H); 4.46 (sext, J=7.1 Hz, 1H); 7.18 (d, J=7.9 Hz, 1H); 7.33 (dd, J=7.9 Hz, J=1.6 Hz, 1H); 7.50 (d, J=1.6 Hz, 1H); 7.81 (s, 1H).

### EXAMPLE 44

### N-cyclopentyl-2-methyl-5-[2-(trifluoromethyl)phenyl]pyrimidin-4-amine

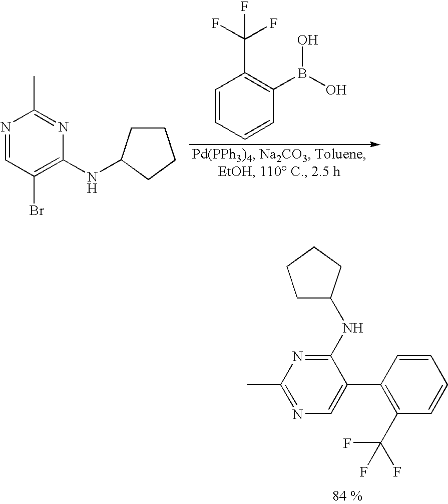

This compound was prepared according to the general procedure described for the example 38.

Aspect: slightly yellow solid Mass obtained: 106 mg

Yield: 84%

M.P.: 80-84° C.

LC-MS: Tᵣ=3.99 min. (100%) (ES-MS: m/z 322.0 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.29-1.43 (m, 2H); 1.50-1.67 (m, 4H); 1.89-2.01 (m, 2H); 2.50 (s, 3H); 4.50 (quint, J=7.1 Hz, 1H); 7.34 (d, J=7.9 Hz, 1H); 7.59-7.72 (m, 3H); 7.84 (d, J=7.9 Hz, 1H).

¹³C-NMR (CD₃OD, 75 MHz) δ: 23.6; 24.6; 32.1; 52.4; 114.1; 124.3 (q, J=272.1 Hz); 126.7; 129.2; 130.2 (q, J=34.6 Hz); 132.8; 133.1; 152.1; 160.0; 166.5.

### EXAMPLE 45

### 5-[2-chloro-4-(trifluoromethyl)phenyl]-N-cyclopentyl-2-methylpyrimidin-4-amine

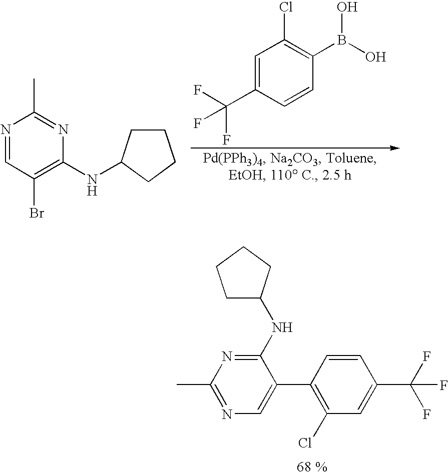

This compound was prepared according to the general procedure described for the example 38 starting from 250 mg (0.98 mmoles, 1.0 eq.) of 5-bromo-N-cyclopentyl-2-methylpyrimidin-4-amine.

Aspect: yellow solid Mass obtained: 238 mg

Yield: 68%

M.P.: 120-132° C.

LC-MS: Tᵣ=4.41 min. (100%) (ES-MS: m/z 356.2 (M); 358.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.38-1.59 (m, 6H); 1.91-2.05 (m, 2H); 2.50 (s, 3H); 4.56 (quint, J=7.1 Hz, 1H); 7.51 (d, J=7.9 Hz, 1H); 7.68-7.74 (m, 2H); 7.84 (s, 1H).

¹³C-NMR (CD₃OD, 75 MHz) δ: 23.9; 24.6; 32.1; 32.4; 52.4; 113.8; 123.6 (q, J=272.1 Hz); 124.6; 126.9; 132.1 (q, J=34.6 Hz); 133.1; 135.4; 137.7; 152.8; 159.3; 166.9.

¹⁹F-NMR (CD₃OD, 282 MHz) δ: −64.8.

### EXAMPLE 46

### N-{4-[4-(cyclopentylamino)-2-methylpyrimidin-5-yl]phenyl}acetamide

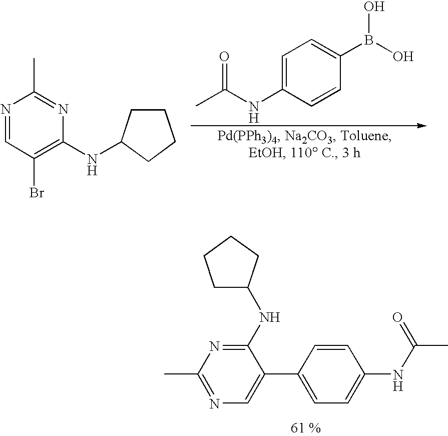

This compound was prepared according to the general procedure described for the example 38. After the flash chromatography, this derivative was recrystallized in 4.5 mL of AcOEt. The resulting solid was filtered off and washed with 1.5 mL of cold AcOEt to give 74 mg of a yellow solid.

Yield: 61%

M.P.: 203-210° C.

LC-MS: Tᵣ=3.08 min. (100%) (ES-MS: m/z 311.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.25-1.37 (m, 2H); 1.60-1.68 (m, 4H); 1.98-2.10 (m, 2H); 2.20 (s, 3H); 2.54 (s, 3H); 4.44 (sext., J=7.2 Hz, 1H); 4.88 (d, J=6.0 Hz, 1H); 7.27 (d, J=9.0 Hz, 2H); 7.62 (d, J=6 Hz, 2H); 7.85 (s, 1H); 8.18 (Is, 1H).

¹³C-NMR (CDCl₃, 300 MHz) δ: 24.1; 24.9; 26.4; 33.5; 52.6; 116.1; 120.9; 129.5; 130.4; 138.3; 153.1; 159.4; 166.7; 168.8.

### EXAMPLE 47

### Cyclopentyl-[2-methyl-5-(5-trifluoromethyl-pyridin-2-yl)-pyrimidin-4-yl]-amine

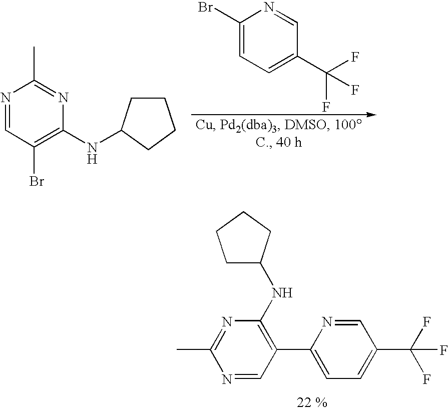

In a parallel synthesis flask, 150 mg (0.59 mmoles, 1.0 eq.) of 5-bromo-N-cyclopentyl-2-methylpyrimidin-4-amine and 132 mg (0.58 mmoles, 1.0 eq.) of 2-bromo-5-trifluoromethylpyridine were dissolved in 1.75 mL of DMSO. 16 mg (0.02 mmoles, 0.03 eq.) of Pd₂(dba)₃ was added followed by 186 mg (2.93 mmoles, 5.0 eq.) of Cu. The mixture was heated at 100° C. under vigourous stirring for 26 h. The mixture was allowed to cool to RT and 132 mg (0.58 mmoles, 1.0 eq.) of 2-bromo-5-trifluoromethylpyridine were added. The solution was heated at 100° C. for additional 14 h. The mixture was allowed to cool to RT and 25 mL of AcOEt were added. The resulting solution was filtered on a pad of Hyflo. The solids were washed five times with 10 mL of AcOEt. The combined filtrates were washed four times with 15 mL of water and once with 20 mL of brine. The organic phase was dried over Na₂SO₄, filtered and evaporated to dryness. The crude compound was purified first by flash chromatography on silica gel and then by preparative TLC to give 41 mg of a white solid.

Yield: 22%

M.P.: 130-133° C.

LC-MS: Tᵣ=4.31 min. (100%) (ES-MS: m/z 323.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.53-1.85 (m, 6H); 2.05-2.16 (m, 2H); 2.50 (s, 3H); 4.56 (quint, J=7.1 Hz, 1H); 8.13 (m, 2H); 8.64 (s, 1H); 8.89 (s, 1H).

¹⁹F-NMR (CD₃OD, 282 MHz) δ: −64.6.

### EXAMPLE 48

### 9-cyclopentyl-2-methyl-7-(trifluoromethyl)-9H-pyrimido[4,5-b]indole

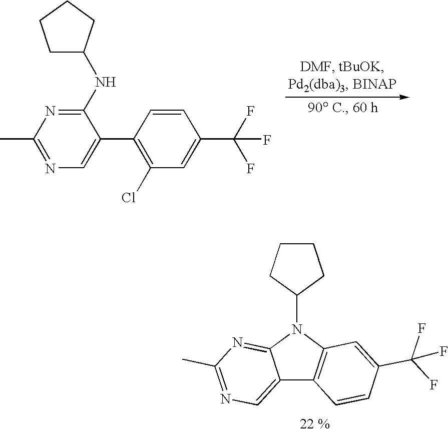

100 mg (0.28 mmoles, 1.0 eq.) of 5-[2-chloro-4-(trifluoromethyl)phenyl]-N-cyclopentyl-2-methylpyrimidin-4-amine, 39 mg (0.34 mmoles, 1.20 eq.) of tBuOK, 9 mg (0.014 mmoles, 0.05 eq.) of rac-BINAP, 13 mg (0.014 mmoles, 0.05 eq.) of Pd₂(dba)₃ were placed in a flask under argon and 1 mL of DMF was added. The reaction was heated at 90° C. for 60 h. The crude mixture was partitioned between 15 mL of water and 20 mL of AcOEt. The aqueous phase was removed and extracted six times with 20 mL of AcOEt. The combined organic layers were washed with 30 mL of brine, dried over Na₂SO₄, filtered and evaporated to dryness. The crude compound was purified by preparative TLC. The resulting crude compound was recrystallized in 0.5 mL of MeOH. The resulting solid was filtered off and washed with 2 mL of cold MeOH to give 20 mg of a white solid.

Yield: 22%

M.P.: 133-134° C.

LC-MS: Tᵣ=4.41 min. (100%) (ES-MS: m/z 320.2 (M+H); 322.2 (M+2+H)) [Column: Nucleosil C-18HD, 4×70 mm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.79-1.92 (m, 2H); 2.08-2.22 (m, 4H); 2.31-2.43 (m, 2H); 2.85 (m, 3H); 5.45-5.56 (quint, J=8.7 Hz, 1H); 7.56 (d, J=8.7 Hz, 1H); 7.78 (s, 1H); 8.16 (d, J=8.16 Hz, 1H); 9.20 (s, 1H).

¹⁹F-NMR (CDCl₃, 282 MHz) δ. −62.0.

### EXAMPLE 49

### 5-(2,1,3-benzoxadiazol-5-yl)-N-cyclopentyl-2-methylpyrimidin-4-amine

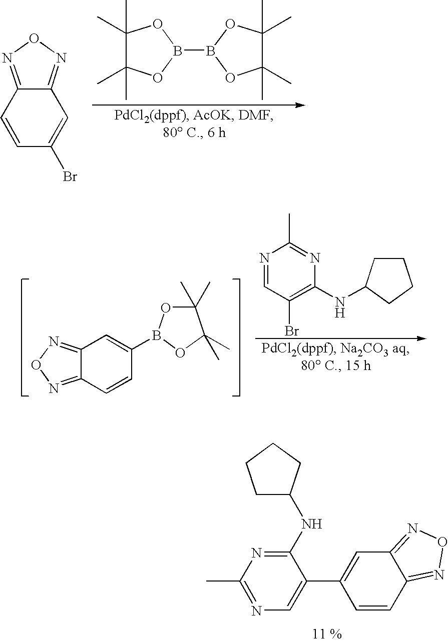

In a flask under argon were placed 200 mg (1.00 mmles, 1.0 eq.) of 5-bromo-2,1,3-benzoxadiazole, 326 mg (1.10 mmoles, 1.10 eq.) of bis(pinacolato)diboron, 986 mg (3.32 mmoles, 3.30 eq.) of AcOK, and 8 mg (0.01 mmoles, 0.01 eq.) of PdCl₂(dppf). 3.5 mL of anhydrous DMF were added and the mixture was stirred at 80° C. for 6 h. It was cooled to RT and 170 mg (0.66 mmoles, 0.66 eq.) of 5-bromo-N-cyclopentyl-2-methylpyrimidin-4-amine, 8 mg (0.01 mmoles, 0.01 eq.) of PdCl₂(dppf) were added followed by a solution of 352 mg (3.32 mmoles, 3.30 eq.) of Na₂CO₃ in 1.41 mL of water. The mixture was heated at 80° C. for 15 h. The experiment was allowed to cool to RT. The solution was partitioned between 35 mL of water and 15 mL of AcOEt. The aqueous phase was extracted two more times with 15 mL of AcOEt. The combined organic layers were washed once with 15 mL of brine, dried over Na₂SO₄, filtered and evaporated to dryness. The crude compound was purified by flash chromatography on silica gel. The crude compound was then recrystallized in 1 mL of hot MeOH. The solution was cooled to RT, evaporated to the half and left overnight in the fridge. The solid was filtered off, washed with 2 mL of Et₂O and dried under high vacuum to give 34 mg of a yellow solid.

Yield: 11%

M.P.: 165-167° C.

LC-MS: Tᵣ=3.61 min. (100%) (ES-MS: m/z 296.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.34-1.45 (m, 2H); 1.62-1.74 (m, 4H); 2.08-2.17 (m, 2H); 2.59 (s, 3H); 4.52 (quint, J=7.0 Hz, 1H); 4.80 (d, J=7.0 Hz, 1H); 7.42 (d, J=9.5 Hz, 1H); 7.84 (s, 1H); 7.96 (d, J=9.5 Hz, 1H); 8.04 (s, 1H).

¹³C-NMR (CDCl₃, 100 MHz) δ: 23.9; 26.1; 32.9; 52.2; 114.2; 115.5; 117.7; 132.9; 138.7; 148.4; 149.3; 153.9; 158.4; 168.1.

### EXAMPLE 50

### N-cyclopentyl-6-iodo-2-methyl-5-[4-(trifluoromethyl)phenyl]pyrimidin-4-amine

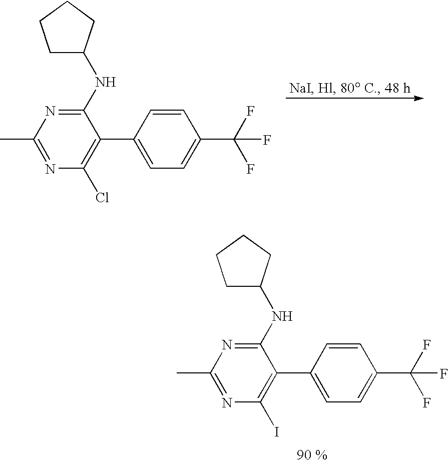

A solution of 150 mg (0.42 mmoles, 1.0 eq.) of 6-chloro-N-cyclopentyl-2-methyl-5-[4-(trifluoromethyl)phenyl]pyrimidin-4-amine in 4 mL of HI was treated with 632 mg (4.22 mmoles, 10 eq.) of NaI and stirred for 48 h at 80° C. The solution was allowed to cool to RT and it was alkalinized with a saturated solution of Na₂CO₃. The aqueous phase was transferred to a separatory funnel and extracted three times with 15 mL of AcOEt. The combined organic layers were washed once with 15 mL of brine, dried over Na₂SO₄, filtered and evaporated to dryness under reduced pressure. The crude compound was purified by flash chromatography on silica gel to give 170 mg of a white solid.

Yield: 90%

M.P.: 140-143° C.

LC-MS: Tᵣ=5.15 min. (100%) (ES-MS: m/z 448.0 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.21-1.30 (m, 2H); 1.54-1.66 (m, 4H); 1.97-2.07 (m, 2H); 2.53 (s, 3H); 4.25 (d, J=7.0 Hz, 1H); 4.37 (sext, J=7.0 Hz, 1H); 7.38 (d, J=8.0 Hz, 2H); 7.79 (d, J=8.0 Hz, 2H).

¹⁹F-NMR (CD₃OD, 282 MHz) δ: −64.8 (—CF₃)

### N-cyclopentyl-6-(trimethylsilylethynyl)-2-methyl-5-[4-(trifluoro-methyl)phenyl]-pyrimidin-4-amine

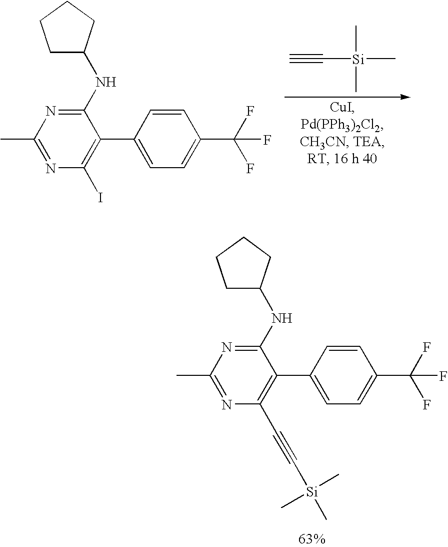

A solution of 200 mg (0.45 mmoles, 1.0 eq.) of N-cyclopentyl-6-iodo-2-methyl-5-[4-(trifluoromethyl)phenyl]pyrimidin-4-amine in 3.7 mL of CH₃CN and 2.77 mL of TEA under argon was treated at ambient temperature with 124 μL (0.89 mmoles, 2.0 eq.) of ethynyltrimethylsilane, 16 mg (0.022 mmoles, 0.05 eq.) of Pd(PPh₃)₂Cl₂ and 5 mg (0.027 mmoles, 0.06 eq.) of CuI. The mixture was stirred at RT for 16 h 40. Solvents were removed under reduced pressure and the crude compound was purified by flash chromatography on silica gel to give 117 mg of a brown solid.

Yield: 63%

M.P.: 151-154° C.

LC-MS: Tᵣ=5.34 min. (100%) (ES-MS: m/z 418.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 0.00 (s, 9H); 1.25-1.33 (m, 2H); 1.56-1.64 (m, 4H); 2.01-2.09 (m, 2H); 2.53 (s, 3H); 4.42 (sext., J=6.3 Hz, 1H); 4.55 (d, J=6.3 Hz, 1H); 7.49 (d, J=9.5 Hz, 2H); 7.74 (J=9.5 Hz, 2H).

¹³C-NMR (CDCl₃, 100 MHz) δ: 0.0; 24.6; 27.2; 34.1; 53.4; 101.6; 102.3; 118.4; 124.9 (q, J=277 Hz); 126.9; 131.8 (m); 139.0; 146.6; 160.1; 168.2.

¹⁹F-NMR (CDCl₃, 377 MHz) δ: −62.7.

### N-cyclopentyl-6-ethynyl-2-methyl-5-[4-(trifluoromethyl)phenyl]pyrimidin-4-amine

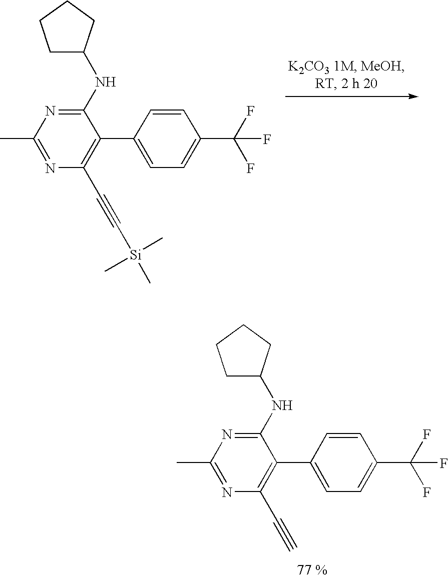

117 mg (0.28 mmoles, 1.0 eq.) of N-cyclopentyl-6-(trimethylsilylethynyl)-2-methyl-5-[4-(trifluoro-methyl)phenyl]-pyrimidin-4-amine were dissolved in 1 mL of MeOH. 1 mL of a 1M aqueous solution of K₂CO₃ was added and the mixture was stirred at RT for 2 h 20. Solvents were removed under reduced pressure and the resulting residue was partitioned between 10 mL of water and 10 mL of DCM. The aqueous phase was extracted two more times with 10 mL of DCM. The combined organic layers were washed once with 10 mL of brine, dried over Na₂SO₄, filtered and evaporated to dryness. The crude compound was purified by flash chromatography on silica gel to give 75 mg of a slightly brown solid.

Yield: 77%

M.P.: 159-162° C.

LC-MS: Tᵣ=4.40 min. (100%) (ES-MS: m/z 346.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.18-1.31 (m, 2H); 1.53-1.66 (m, 4H); 1.97-2.08 (m, 2H); 2.53 (s, 3H); 2.99 (s, 1H); 4.39 (sext., J=7.1 Hz, 1H); 4.51 (d, J=7.1 Hz, 1H); 7.46 (d, J=8.0 Hz, 2H); 7.71 (d, J=8.0 Hz, 2H).

¹³C-NMR (CDCl₃, 75 MHz) δ: 23.9; 26.5; 33.4; 52.8; 81.0; 82.0; 117.4; 124.3 (q, J=272 Hz); 126.2; 130.8 (m); 137.7; 144.9; 159.7; 167.5.

¹⁹F-NMR (CDCl₃, 282 MHz) δ: −63.5.

### EXAMPLE 51

### 6-chloro-N-cyclopentyl-2-methyl-5-(4-nitrophenyl)pyrimidin-4-amine

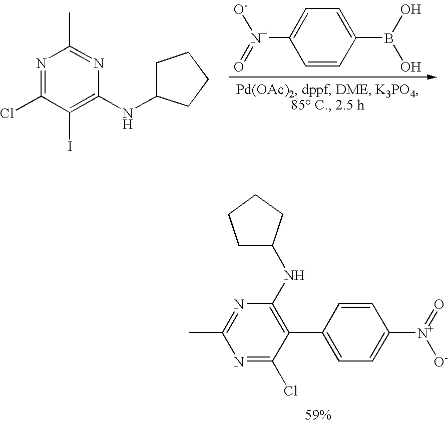

This compound was prepared according to the general procedure described for the example 12 starting from 1.5 g (4.44 mmoles) of 6-chloro-N-cyclopentyl-5-iodo-2-methylpyrimidin-4-amine. After the flash chromatography, a recrystallisation in 37.5 mL of MeOH was performed to give 872 mg of a slightly yellow solid.

Yield: 59%

M.P.: 188-192° C.

LC-MS: Tᵣ=5.74 min. (100%) (ES-MS: m/z 333.2 (M); 335.2 (M+2)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.24-1.34 (m, 2H); 1.58-1.68 (m, 4H); 2.01-2.12 (m, 2H); 2.56 (s, 3H); 4.39-4.49 (m, 2H); 7.53 (d, J=9.5 Hz, 2H); 8.37 (d, J=9.5 Hz, 2H).

¹³C-NMR (CDCl₃, 100 MHz) δ: 23.6; 25.9; 33.1; 53.1; 111.5; 124.9; 131.5; 140.0; 147.9; 156.4; 160.3; 167.9.

### 5-(4-aminophenyl)-N-cyclopentyl-2-methylpyrimidin-4-amine

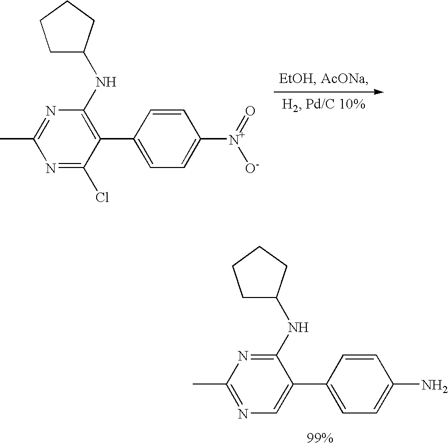

This compound was prepared according to the general procedure described for the example 12 starting from 800 mg (2.40 mmoles, 1.0 eq.) of 6-chloro-N-cyclopentyl-2-methyl-5-(4-nitrophenyl)pyrimidin-4-amine. After removal of the catalyst by filtration, the crude compound was dissolved in 100 mL of AcOEt. The organic phase was washed twice with 80 mL of a saturated solution of Na₂CO₃, once with 80 mL of brine, dried over Na₂SO₄, filtered and evaporated to dryness to give 638 mg of a yellow solid.

Yield: 99%

M.P.: 120-124° C.

LC-MS: Tᵣ=2.40 min. (100%) (ES-MS: m/z 269.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 400 MHz) δ: 1.21-1.34 (m, 2H); 1.51-1.66 (m, 4H); 1.95-2.05 (m, 2H); 2.53 (s, 3H); 4.00 (Is, 2H); 4.42 (sext.; J=7.0 Hz, 1H); 4.93 (d, J=7.0 Hz, 1H); 6.71 (d, J=8.4 Hz, 2H); 7.06 (d, J=8.4 Hz, 2H); 7.84 (s, 1H).

¹³C-NMR (CDCl₃, 100 MHz) δ: 23.6; 26.2; 33.1; 52.1; 115.7; 116.7; 123.9; 129.8; 146.9; 153.1; 159.7; 166.2.

### N-cyclopentyl-5-[4-(3,3-diethyltriaz-1-en-1-yl)phenyl]-2-methyl-pyrimidin-4-ylamine

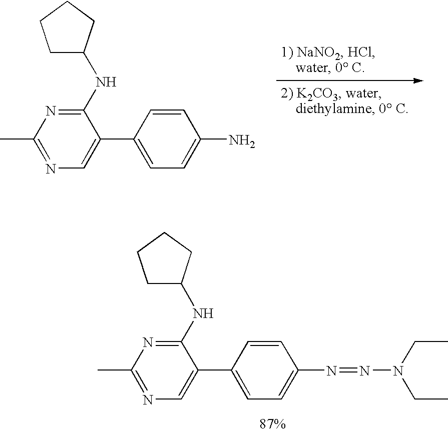

To a solution containing 100 mg (0.37 mmoles, 1.0 eq.) of 5-(4-aminophenyl)-N-cyclopentyl-2-methylpyrimidin-4-amine in a mixture of 300 μL of concentrated HCl and 300 μL of water was added dropwise a solution of 28 mg (0.40 mmoles, 1.08 eq.) of sodium nitrite in 100 μL of water at 0° C. The reaction mixture was stirred at 0° C. for an additional 30 min. and was then transferred to a solution containing 232 mg (1.68 mmoles, 4.50 eq.) of K₂CO₃ and 174 μL (1.68 mmoles, 4.50 eq) of diethylamine in 746 μL of water. It was stirred 1 h at 0° C. 10 mL of Et₂O were added and the solution was transferred to a separatory funnel. The organic phase was removed and the aqueous phase was extracted two more times with 10 mL of Et₂O. The aqueous phase was alkalinized with a saturated solution of Na₂CO₃ and it was extracted three more times with Et₂O. The combined organic layers were washed once with 10 mL of a saturated solution of Na₂CO₃, once with 10 mL of water and once with 10 mL of brine, dried over Na₂SO₄, filtered and evaporated to dryness. The crude compound was purified by flash chromatography on silica gel to give 114 mg of a colourless oil.

Yield: 87%

Mass Spectrum (triazene): ES-MS: m/z 353.2 (M+H)

¹H-NMR (CDCl₃, 300 MHz) δ: 1.23-1.35 (m, 8H); 1.55-1.64 (m, 4H); 1.98-2.07 (m, 2H); 2.52 (s, 3H); 3.75 (q, J=7.0 Hz, 4H); 4.42 (sext, J=7.0 Hz, 1H); 4.92 (d, J=6 Hz, 1H); 7.26 (d, J=9 Hz, 2H); 7.45-7.48 (d, J=9 Hz, 2H); 7.89 (s, 1H).

¹³C-NMR (CDCl₃, 75 MHz) δ: 24.0; 26.5; 33.5; 52.5; 116.5; 121.5; 129.4; 131.2; 151.2; 153.1; 159.5; 166.5.

### N-cyclopentyl-5-(4-iodophenyl)-2-methylpyrimidin-4-amine

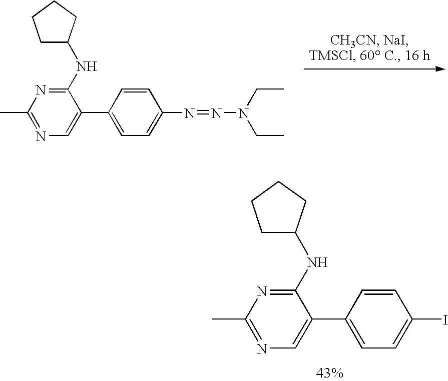

To a solution of 114 mg (0.32 mmoles, 1.0 eq.) of N-cyclopentyl-5-[4-(3,3-diethyltriaz-1-en-1-yl)phenyl]-2-methyl-pyrimidin-4-ylamine and 194 mg (1.29 mmoles, 4.0 eq.) of NaI in 3.23 mL of acetonitrile was added 102 μL (0.81 mmoles, 2.5 eq.) of TMSCl. This mixture was heated at 60° C. for 5 min. 194 mg (1.29 mmoles, 4.0 eq.) of NaI were added. The mixture was heated at 60° C. for additional 55 min. 102 μL (0.81 mmoles, 2.5 eq.) of TMSCl were added and the mixture was heated at 60° C. for additional 30 min. 194 mg (1.29 mmoles, 4.0 eq.) of NaI were added and the mixture was stirred at 60° C. for additional 1 h 30. 95 mg (0.647 mmoles, 2.0 eq.) of NaI were added and the mixture was stirred at 60° C. overnight. The solution was cooled to RT and 30 mL of a saturated solution of Na₂CO₃ were added. The aqueous layer was extracted with Et₂O. Combined organic layers were washed once with water, once with brine, dried over Na₂SO₄, filtered and evaporated to dryness. The crude compound was purified by preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 mL/min.) to give 53 mg of a white solid.

Yield: 43%

M.P.: 110-113° C.

LC-MS: Tᵣ=4.23 min. (100%) (ES-MS: m/z 380.0 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.24-1.38 (m, 2H); 1.56-1.70 (m, 4H); 2.00-2.12 (m, 2H); 2.54 (s, 3H); 4.45 (sext, J=7.1 Hz, 1H); 4.79 (d, J=7.1 Hz, 1H); 7.08 (d, J=7.9 Hz, 2H); 7.79 (d, J=7.9 Hz, 2H); 7.87 (s, 1H).

¹³C-NMR (CDCl₃, 75 MHz) δ: 23.9; 26.5; 33.4; 52.4; 94.1; 115.4; 131.1; 134.7; 139.0; 153.4; 159.0; 167.2.

### Diethyl {4-[4-(cyclopentylamino)-2-methylpyrimidin-5-yl]phenyl}-phosphonate

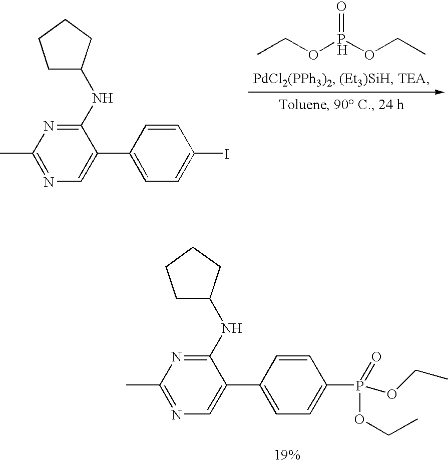

In a parallel synthesis flask under argon were placed 9 mg (0.013 mmoles, 0.05 eq.) of PdCl₂(PPh₃)₂. Then a solution of 2 μL (0.013 mmoles, 0.05 eq.) of TES in 150 μL of toluene was added. The mixture was stirred at 90° C. for 10 min to yield a black solution. 37 μL (0.29 mmoles, 1.10 eq.) of diethyl hydrogen phosphate, 100 mg (0.264 mmoles, 1.0 eq.) of N-cyclopentyl-5-(4-iodophenyl)-2-methylpyrimidin-4-amine, 44 μL (0.32 mmoles, 1.2 eq.) of TEA, and 150 μL of toluene were added. The mixture was heated at 90° C. for 24 h. The mixture was cooled to RT and solvents were removed under reduced pressure. The resulting oil was purified by preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 mL/min.) to give 19 mg of a yellow oil.

Yield: 19%

LC-MS: Tᵣ=3.61 min. (100%) (ES-MS: m/z 390.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.39 (t, J=8.0 Hz, 6H); 1.47-1.54 (m, 2H); 1.63-1.76 (m, 4H); 2.03-2.09 (m, 2H); 2.54 (s, 3H); 4.15-4.24 (m, 4H); 4.59 (quint, J=8.0 Hz, 1H); 7.63 (dd, J¹=8.0 Hz, J²=4.0 Hz, 2H); 7.89 (s, 1H); 7.94 (dd, J¹=14 Hz, J²=10 Hz, 2H).

³¹P-NMR (CD₃OD, 400 MHz) δ: 18.6.

### {4-[4-(cyclopentylamino)-2-methylpyrimidin-5-yl]phenyl}-phosphonic acid

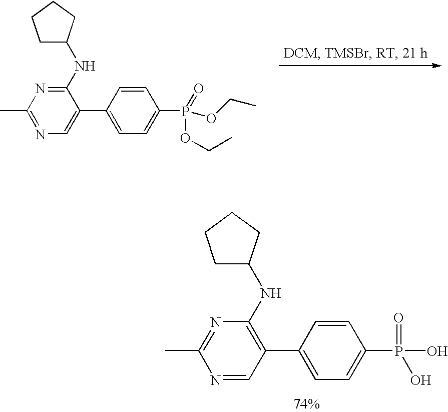

19 mg (0.049 mmoles, 1.0 eq.) of Diethyl {4-[4-(cyclopentylamino)-2-methylpyrimidin-5-yl]phenyl}-phosphonate were dissolved in 1 mL of DCM. 32 μL (0.24 mmoles, 5.0 eq.) of TMSBr were added and the mixture was stirred at RT for 21 h. Volatiles were removed under reduced pressure and the crude compound was purified by preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 ml/min.) to give 12 mg of a white solid.

Yield: 74%

M.P.: 254-259° C.

LC-MS: Tᵣ=1.99 min. (100%) (ES-MS: m/z 334.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 400 MHz) δ: 1.53-1.79 (m, 6H); 2.03-2.09 (m, 2H); 2.67 (s, 3H); 4.72 (quint, J=8.0 Hz, 1H); 7.44 (dd, J₁=8.0 Hz, J₂=4.0 Hz, 2H); 7.54 (s, 1H); 7.97 (dd, J₁=12.0 Hz, J₂=8.0 Hz, 2H).

### EXAMPLE 52

### 6-chloro-2-methylpyrimidin-4-ol

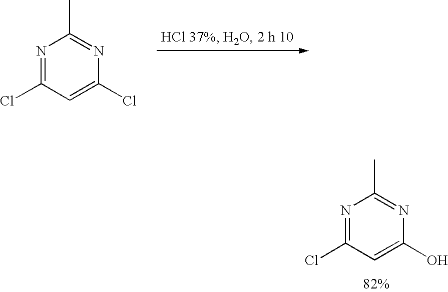

10 g (61.35 mmoles, 1.0 eq.) of 4,6-dichloro-2-methylpyrimidine were suspended in 106 mL of water. 43 mL of concentrated HCl were added and the solution was refluxed for 2 h 10. Solvents were removed under reduced pressure and the crude compound was recrystallized in 200 mL of water. The solution was left one night in the fridge. The resulting solid was filtered and washed with water. It was dried under high vacuum at 50° C. Mother liquors were evaporated and a second recrystallisation was performed in 45 mL of water. The solution was left overnight in the fridge. The solid was filtered off and was dried under high vacuum at 50° C. The two batches were mixed to give 7.29 g of white needles.

Yield: 82%

M.P.: 230-232° C.

LC-MS: Tᵣ=2.57 min. (100%) (ES-MS: m/z 145.0 (M+H); 147.0 (M+2+H)) [Column: Nucleosil C-18HD, 4×70 mm, 3 μm, gradient CH₃CN/H₂O/TFA 0.05%: 5-100% CH₃CN (6 min.), 100% CH₃CN (1.5 min.), flow: 1 mL/min].

¹H-NMR (DMSO-D₆, 400 MHz) δ: 2.31 (s, 3H); 6.35 (s, 1H).

¹³C-NMR (DMSO-D₆, 100 MHz) δ: 21.4; 111.0; 158.0; 161.3; 162.2.

### 6-chloro-5-iodo-2-methylpyrimidin-4-ol

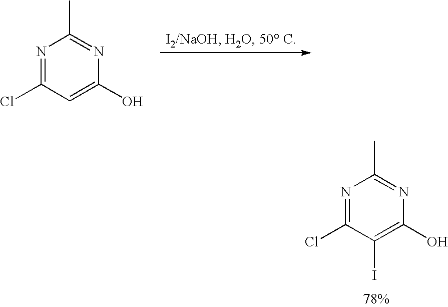

6 g (41.50 mmoles, 1.0 eq.) of 6-chloro-2-methylpyrimidin-4-ol and 1.99 g (99.61 mmoles, 2.40 eq.) of NaOH were dissolved in 39.5 mL of water. Then 12.43 g (90.48 mmoles, 2.18 eq.) of iodine were added and the solution was heated at 50° C. for 3 h 20. 5.27 g (20.76 mmoles, 0.5 eq.) of iodine and 1 g (49.81 mmoles, 0.6 eq.) of NaOH were added and the mixture was heated at 50° C. for additional 24 h 40. 5.27 g (20.76 mmoles, 0.5 eq.) of iodine and 1 g (49.81 mmoles, 0.6 eq.) of NaOH were added and the mixture was heated for additional 17 h. The solution was cooled to RT and was acidified with AcOH. The solid was filtered off and washed with water. It was then recrystallized in 170 mL of EtOH. The solution was left overnight in the fridge. The resulting solid was filtered off, washed with EtOH and dried under high vacuum at 40° C. to give 8.74 g of a brown solid.

Yield: 78%

M.P.: decomposition at 263° C.

LC-MS: Tᵣ=3.62 min. (100%) (ES-MS: m/z 271.0 (M+H); 272.8 (M+2+H)) [Column: Nucleosil C-18HD, 4×70 mm, gradient CH₃CN/H₂O/TFA 0.05%: 5-100% CH₃CN (6 min.), 100% CH₃CN (1.5 min.), flow: 1 mL/min].

¹H-NMR (DMSO-D₆, 300 MHz) δ: 2.24 (s, 3H).

### 3-benzyl-6-chloro-5-iodo-2-methylpyrimidin-4(3H)-one

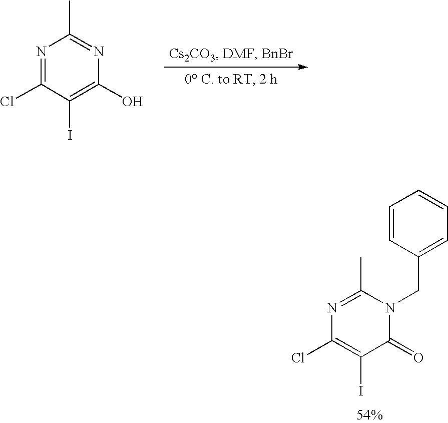

To a cooled solution of 1 g (3.70 mmoles, 1.0 eq.) of 6-chloro-5-iodo-2-methylpyrimidin-4-ol in 11 mL of DMF, 1.33 g (4.07 mmoles, 1.10 eq.) of Cs₂CO₃ were added. The reaction mixture was stirred at 0° C. for 30 min. Then, 483 μL (4.07 mmoles, 1.10 eq.) of BnBr were added dropwise at 0° C. and the solution was allowed to reach RT and was stirred for 2 h. The crude mixture was partitioned between 110 mL of water and 50 mL of AcOEt. The aqueous phase was decanted and extracted three more times with 50 mL of AcOEt. The combined organic layers were washed once with 50 mL of brine, dried over Na₂SO₄, filtered and evaporated to dryness. The crude compound was purified by flash chromatography on silica gel to give 716 mg of a yellow solid.

Yield: 54%

M.P.: 135-148° C.

LC-MS: Tᵣ=4.98 min. (100%) (ES-MS: m/z 383.0 (M+Na); 385.0 (M+2+Na)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 ml/min].

¹H-NMR (CDCl₃, 300 MHz) δ. 2.48 (s, 3H); 5.31 (s, 2H); 7.17-7.36 (m, 5H).

¹³C-NMR (CDCl₃, 75 MHz) δ: 22.9; 49.5; 87.5; 127.1; 128.5; 129.3; 134.2; 159.2; 160.7; 161.0.

### 3-benzyl-6-chloro-2-methyl-5-[4-(trifluoromethyl)phenyl]pyrimidin-4(3H)-one

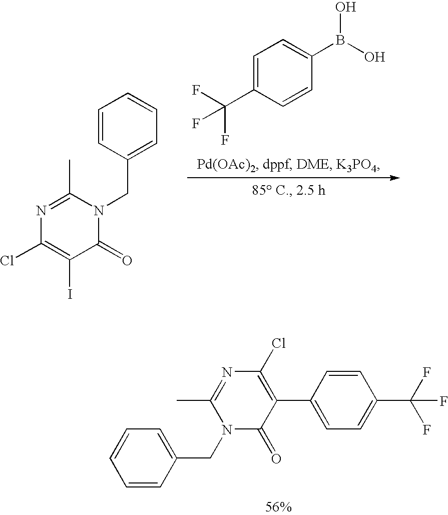

This compound was prepared according to the general procedure described for the example 12 starting from 600 mg (4.44 mmoles, 1.0 eq.) of 3-benzyl-6-chloro-5-iodo-2-methylpyrimidin-4(3H)-one. After the extraction, a preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 mL/min.) was performed to give 352 mg of a white solid.

Yield: 56%

M.P.: 114-117° C.

LC-MS: Tᵣ=5.91 min. (100%) (ES-MS: m/z 379.0 (M+H); 401.0 (M+2+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 2.50 (s, 3H); 5.26 (s, 2H); 7.20-7.35 (m, 5H); 7.58 (d, J=9.0 Hz, 2H); 7.69 (d, J=6.0 Hz, 2H).

¹³C-NMR (CDCl₃, 75 MHz) δ: 23.4; 48.4; 121.7; 124.3 (q, J=271.6 Hz); 125.3; 127.1; 128.4; 129.3; 130.4 (q, J=25.6 Hz); 131.0; 134.6; 136.4; 154.5; 159.4; 161.4.

¹⁹F-NMR (CDCl₃, 282 MHz) δ: −63.3.

### 3-benzyl-6-chloro-2-methyl-5-[4-(trifluoromethyl)phenyl]pyrimidin-4(3H)-one

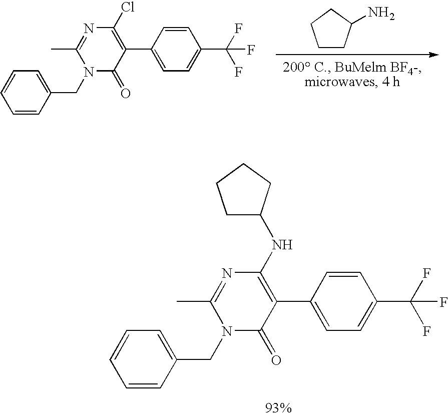

100 mg (0.26 mmoles, 1.0 eq.) of 3-benzyl-6-chloro-2-methyl-5-[4-(trifluoromethyl)phenyl]pyrimidin-4(3H)-one were dissolved in 2 mL of cyclopentyl amine. Two drops of BuMelm BF₄⁻ were added and the mixture was heated at 200° C. under microwaves irradiation for 4 h. Solvents were removed under reduced pressure and the crude compound was purified by flash chromatography on silica gel to give 105 mg of a white foam.

Yield: 93%

LC-MS: Tᵣ=6.78 min. (100%) (ES-MS: m/z 428.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.29-1.42 (m, 2H); 1.52-1.69 (m, 4H); 1.92-2.00 (m, 2H); 2.41 (s, 3H); 4.47 (sext, J=7.2 Hz, 1H); 5.30-5.36 (m, 3H); 7.19-7.35 (m, 5H); 7.55 (d, J=9.0 Hz, 2H); 7.73 (d, J=6.0 Hz, 2H).

¹³C-NMR (CD₃OD, 75 MHz) δ: 22.5; 23.6; 32.9; 46.6; 53.2; 96.7; 124.6 (q, J=262.1 Hz); 125.6; 125.7; 126.3; 127.3; 128.7; 129.0; 131.6; 136.7; 138.2; 158.2; 159.3; 161.8.

¹⁹F-NMR (CD₃OD, 282 MHz) δ: −64.6.

### 6-(cyclopentylamino)-2-methyl-5-[4-(trifluoromethyl)phenyl]-pyrimidin-4-ol hydrochloride

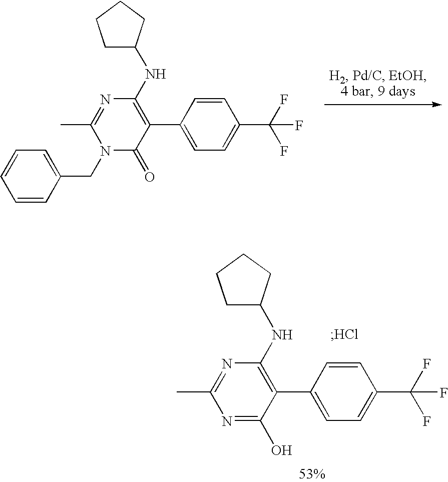

50 mg (0.12 mmoles, 1.0 eq.) of 3-benzyl-6-chloro-2-methyl-5-[4-(trifluoromethyl)phenyl]pyrimidin-4(3H)-one were dissolved in 5 mL of EtOH. 15 mg of Pd/C 10% were added and the solution was hydrogenated at 4 bar and at RT for 9 days. The catalyst was removed by filtration and solvents were removed by evaporation under reduced pressure. The crude compound was purified by preparative TLC first and then it was dissolved in 5 mL of AcOEt and 74 μL of a solution of HCl 2M in Et₂O were added. The solution was then evaporated to approximately 1 mL. The resulting solid was filtered off and washed with 2 mL of Et₂O to give 23 mg of a white solid.

Yield: 53%

LC-MS: Tᵣ=5.18 min. (100%) (ES-MS: m/z 338.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.28-1.39 (m, 2H); 1.61-1.69 (m, 4H); 1.98-2.04 (m, 2H); 2.62 (s, 3H); 4.41 (m, 1H); 5.53 (m, 1H); 7.43 (d, J=6.0 Hz, 2H); 7.75 (d, J=9.0 Hz, 2H).

¹⁹F-NMR (CDCl₃, 282 MHz) δ: −63.6.

### EXAMPLE 53

### Methyl 2-chloro-5-(trifluoromethyl)benzoate

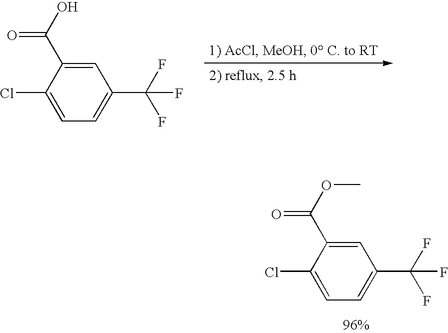

1.81 mL (25.38 mmoles, 2.85 eq.) of acetyl chloride were added dropwise to 20 mL of MeOH at 0° C. The solution was stirred at 0° C. for 10 min. Then 2 g (8.91 mmoles, 1.0 eq.) of 2-chloro-5-trifluoromethylbenzoic acid were added. The mixture was allowed to reach RT and was then refluxed for 2.5 h. The mixture was allowed to cool to RT and solvents were removed under reduced pressure. The crude compound was dissolved in 100 mL of AcOEt and the organic phase was washed twice with 50 mL of a saturated aqueous solution of Na₂CO₃, once with 50 mL of brine, dried over Na₂SO₄, filtered and evaporated to dryness to give 2.05 g of a colourless liquid.

Yield: 96%

LC-MS: Tᵣ=5.44 min. (100%) (no ionization) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 3.96 (s, 3H); 7.57 (d, J=9.0 Hz, 1H); 7.65 (dd, J¹=9.0 Hz, J²=3.0 Hz, 1H); 8.09 (d, J=3.0 Hz, 1H).

### Methyl 5-trifluoromethyl-2-(4,4,5,5-tetramethyl-1,3,2-dioxaborolan-2-yl-benzoate

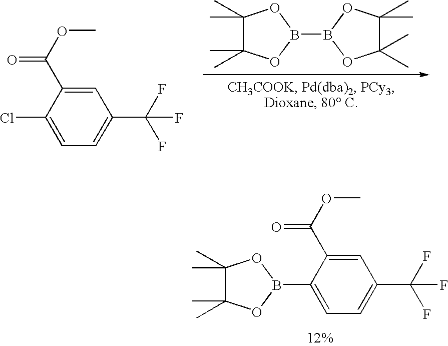

A flask under argon was charged with 145 mg (0.25 mmoles, 0.06 eq.) of Pd(dba)₂, 170 mg (0.59 mmoles, 0.14 eq.) of P(Cy)₃. 25 mL of dioxane were added and the mixture was stirred 30 min. at RT. 1.19 g (4.61 mmoles, 1.10 eq.) of bis(pinacolato)diboran, 617 mg (6.29 mmoles, 1.50 eq.) of AcOK and 1 g (4.19 mmoles, 1.0 eq.) of Methyl 2-chloro-5-(trifluoromethyl)benzoate were added successively. The mixture was then heated at 80° C. for 20 h. 25 mL of water were added followed by 15 mL of AcOEt. The mixture was transferred to a separatory funnel and the aqueous phase was extracted three more times with 15 mL of AcOEt. The combined organic layers were washed with 15 mL of brine, dried over Na₂SO₄, filtered and evaporated to dryness. The crude compound was purified by flash chromatography on silica gel to give 161 mg of a yellow solid.

Yield: 12%

M.P.: 54-59° C.

LC-MS: Tᵣ=6.18 min. (100%) (ES-MS: m/z 231.0) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.42 (s, 12H); 3.96 (s, 3H); 7.68 (d, J=9.0 Hz, 1H); 7.87 (d, J=6.0 Hz, 1H); 8.17 (s, 1H).

### 5-cyclopentyl-3-methyl-8-(trifluoromethyl)pyrimido[4,5-c]-isoquinolin-6(5H)-one

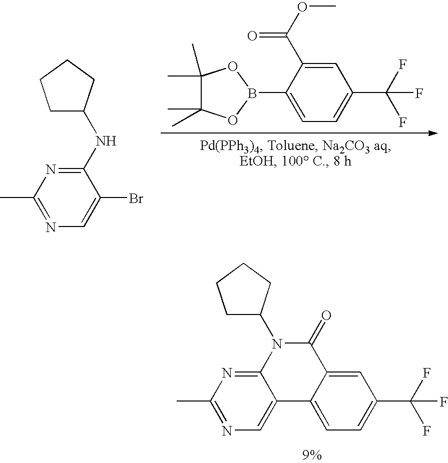

This compound was prepared according to the general procedure described for the example 38 starting from 118 mg (0.46 mmoles, 1.0 eq.) of 5-bromo-N-cyclopentyl-2-methylpyrimidin-4-amine.

Aspect: white solid Mass obtained: 14 mg

Yield: 9%

M.P.: 137-141° C.

LC-MS: Tᵣ=6.36 min. (100%) (ES-MS: m/z 280.0 (M-cyclopentyl); 348.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.69-1.80 (m, 2H); 1.91-2.02 (m, 2H); 2.10-2.19 (m, 2H); 2.21-2.39 (m, 2H); 2.82 (s, 3H); 6.13 (quint, J=6.7 Hz, 1H); 7.98 (dd, J¹=9.0 Hz, J²=3.0 Hz, 1H); 8.33 (d, J=9.0 Hz, 1H); 8.78 (s, 1H); 9.39 (s, 1H).

¹⁹F-NMR (CDCl₃, 282 MHz) δ: −63.5.

### EXAMPLE 54

### 5-bromo-2-(trifluoromethyl)pyrimidine

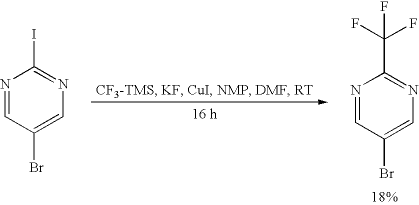

A mixture of 1.77 g (30.35 mmoles, 1.33 eq.) of KF and 5.79 g (30.35 mmoles, 1.33 eq.) of CuI were stirred and heated together using a heat gun under vacuum (1 mm) for 20 min. After cooling, 20 mL of DMF and 20 ml of NMP were added followed by 4.1 mL (27.38 mmoles, 1.20 eq.) of CF₃-TMS and 6.5 g (22.82 mmoles, 1.0 eq.) of 5-bromo-2-iodopyrimidine. The mixture was stirred at RT for 16 h. The crude mixture was poured onto 200 mL of NH₄OH 6N and the aqueous phase was extracted six times with 50 mL of AcOEt. The combined organic layers were washed three times with 50 mL of a saturated solution of Na₂CO₃, once with 50 mL of brine, dried over Na₂SO₄, filtered and evaporated to dryness. The crude compound was purified by flash chromatography on silica gel to give 940 mg of a white solid.

Yield: 18%

M.P.: 33-39° C.

LC-MS: Tᵣ=4.32 min. (100%) (no ionization)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 8.93 (s, 2H).

¹⁹F-NMR (CDCl₃, 282 MHz) δ: −70.8.

### N-cyclopentyl-2-methyl-2′-(trifluoromethyl)-5,5′-bipyrimidin-4-amine hydrochloride

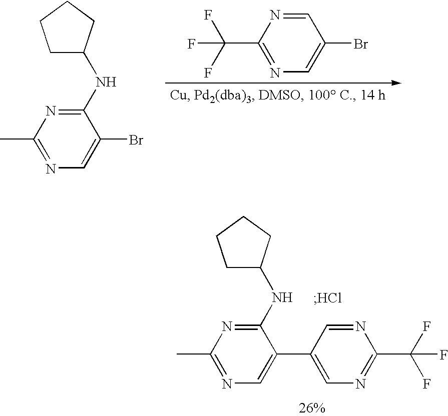

In a parallel synthesis flask under argon, 100 mg (0.39 mmoles, 1.0 eq.) of 5-bromo-N-cyclopentyl-2-methylpyrimidin-4-amine and 177 mg (0.78 mmoles, 2.0 eq.) of 5-bromo-2-(trifluoromethyl)pyrimidine were dissolved in 1.75 mL of DMSO. 11 mg (0.012 mmoles, 0.03 eq.) of Pd₂(dba)₃ were added followed by 186 mg (1.95 mmoles, 5.0 eq.) of Cu. The mixture was heated at 100° C. under vigorous stirring for 14 h. The mixture was allowed to cool to RT and was poured into 20 mL of NH₄OH 27%. This aqueous solution was extracted four times with 10 mL of AcOEt. The combined organic layers were washed once with 10 mL of brine, dried over Na₂SO₄, filtered and evaporated to dryness. The crude mixture was purified by flash chromatography on silica gel. The resulting compound was dissolved in 5 ml of Et₂O and 140 μL of a solution of HCl 2M in Et₂O were added. The solid was filtered off and washed with 1 mL of Et₂O to give 36 mg of a white solid.

Yield: 26%

M.P.: 249-254° C.

LC-MS: Tᵣ=3.55 min. (100%) (ES-MS: m/z 324.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.50-1.80 (m, 6H); 2.02-2.13 (m, 2H); 2.68 (s, 3H); 4.73 (quint, J=7.5 Hz, 1H); 8.16 (s, 1H); 9.07 (s, 2H).

¹⁹F-NMR (CD₃OD, 282 MHz) δ: −72.5.

### EXAMPLE 55

### N-cyclopentyl-2-methyl-5-{[4-(trifluoromethyl)phenyl]ethynyl}-pyrimidin-4-amine

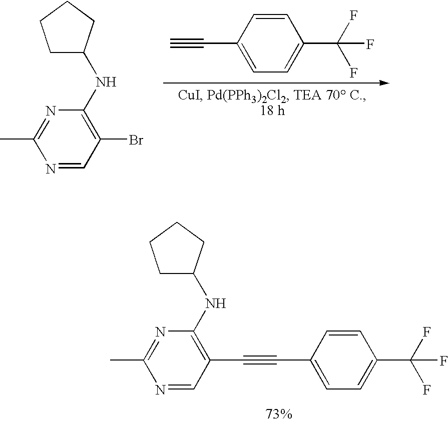

A solution of 100 mg (0.39 mmoles, 1.0 eq.) of 5-bromo-N-cyclopentyl-2-methylpyrimidin-4-amine in 1 mL of TEA was treated at RT with 127 μL (0.78 mmoles, 2.0 eq.) of 4′-trifluoromethylphenyl acetylene, 4 mg (0.023 mmoles, 0.06 eq.) of CuI and 14 mg (0.02 mmoles, 0.05 eq.) of Pd(PPh₃)₂Cl₂. This solution was stirred at 70° C. for 17 h50. The mixture was cooled to RT and was dissolved in 50 mL of AcOEt. The organic phase was washed twice with 25 mL of water, once with 25 mL of brine, dried over Na₂SO₄, filtered and evaporated to dryness. The crude compound was purified by flash chromatography on silica gel to give 99 mg of a brown solid.

Yield: 73%

M.P.: 90-93° C.

LC-MS: Tᵣ=4.67 min. (100%) (ES-MS: m/z 346.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.41-1.52 (m, 2H); 1.62-1.79 (m, 4H); 2.07-2.18 (m, 2H); 2.53 (s, 3H); 4.48 (sext, J=6.6 Hz, 1H); 5.36 (d, J=6.0 Hz, 1H); 7.56 (d, J=9.0 Hz, 2H); 7.60 (d, J=9.0 Hz, 2H); 8.23 (s, 1H).

¹³C-NMR (CDCl₃, 75 MHz) δ: 24.1; 26.9; 33.7; 52.6; 84.6; 96.5; 98.5; 124.3 (q, J=325.2 Hz); 125.6; 130.5 (q, J=33.2 Hz); 131.8; 157.3; 160.8; 167.5.

¹⁹F-NMR (CDCl₃, 282 MHz) δ: −63.6.

### EXAMPLE 56

### N-cyclopentyl-2-methyl-5-{(Z)-2-[4-(trifluoromethyl)phenyl]vinyl}-pyrimidin-4-amine

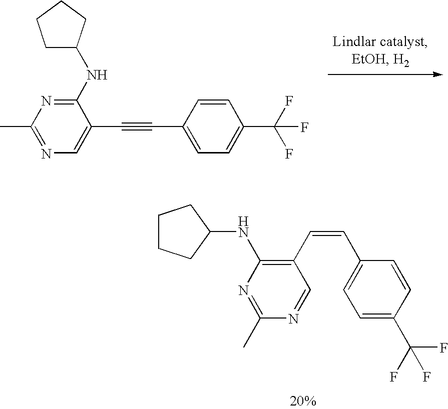

25 mg (0.072 mmoles, 1.0 eq.) of N-cyclopentyl-2-methyl-5-{[4-(trifluoromethyl)phenyl]ethynyl}-pyrimidin-4-amine were dissolved in 2 mL of EtOH. 2 mg of Lindlar catalyst were added and the solution was hydrogenated at atmospheric pressure and at RT for 1 h. Catalyst was removed by filtration and solvents were evaporated under reduced pressure. The resulting crude compound was purified by preparative HPLC (Column: Waters C18-ODB, 19×50 mm, 5 μm, gradient CH₃CN/H₂O/HCOOH 0.05%: 5-100% CH₃CN (10 min.), 100% CH₃CN (2.5 min.), flow: 20 mL/min.) to give 5 mg of a colourless oil.

LC-MS: Tᵣ=4.36 min. (100%) (ES-MS: m/z 348.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CD₃OD, 300 MHz) δ: 1.30-1.40 (m, 2H); 1.54-1.65 (m, 4H); 1.90-1.98 (m, 2H); 2.42 (s, 3H); 4.43 (quint, J=7.5 Hz, 1H); 6.51 (d, J=12.0 Hz, 1H); 6.86 (d, J=12.0 Hz, 1H); 7.36 (d, J=9.0 Hz, 2H); 7.54 (d, J=9.0 Hz, 2H); 7.66 (s, 1H).

### EXAMPLE 57

### N-cyclopentyl-2-methyl-5-{2-[4-(trifluoromethyl)phenyl]ethyl}-pyrimidin-4-amine

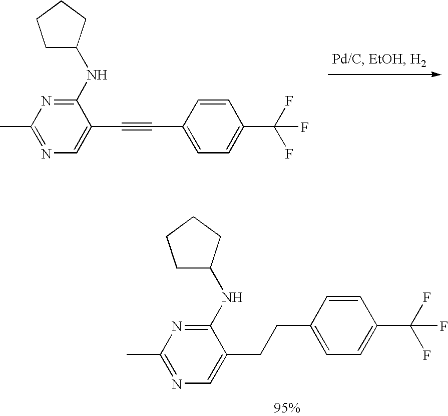

5 mg (0.072 mmoles, 1.0 eq.) of N-cyclopentyl-2-methyl-5-{[4-(trifluoromethyl)phenyl]ethynyl}-pyrimidin-4-amine were dissolved in 2 mL of EtOH. 2 mg of Pd/C 10% were added and the solution was hydrogenated at atmospheric pressure and at RT for 1 h. Catalyst was removed by filtration and solvents were evaporated under reduced pressure. The resulting crude compound was purified by flash chromatography on silica gel to give 24 mg of a yellow oil.

Yield: 95%

LC-MS: Tᵣ=4.56 min. (100%) (ES-MS: m/z 350.2 (M+H)) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

¹H-NMR (CDCl₃, 300 MHz) δ: 1.20-1.33 (m, 2H); 1.58-72 (m, 4H); 2.01-2.11 (m, 2H); 2.49 (s, 3H); 2.61 (t, J=7.5 Hz, 2H); 2.94 (t, J=7.5 Hz, 2H); 4.29 (d, J=6.0 Hz, 1H); 4.39 (sext, J=7.2 Hz, 1H); 7.24 (d, J=9.0 Hz, 2H); 7.53 (d, J=9.0 Hz, 2H); 7.79 (s, 1H).

¹⁹F-NMR (CDCl₃, 282 MHz) δ: −63.1.

### EXAMPLE 58

### Cyclohexyl-[2-methyl-5-(4-trifluoromethyl-phenyl)-pyrimidin-4-yl]-amine

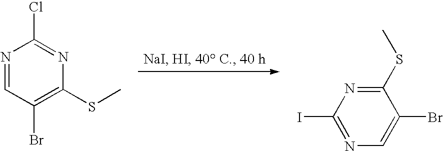

To a suspension of 100 mg of 5-bromo-2-chloro-4-methylsulfanyl-pyrimidine in 1 ml of HI (47%) 125 mg of sodium iodide were added and the mixture was heated for 40 h. During this time two additional portions of sodium iodide were added. After completion of the reaction the mixture was poured into 20 ml of a saturated solution of Na₂CO₃, the organic phase was extracted with ethyl acetate and the combined organic layers were washed with brine and dried over Na₂SO₄. Evaporation of the solvent gave a crude product which was purified by flash chromatography on silica gel with hexane/ethyl acetate 97.5:2.5, yielding 83 mg of 5-bromo-2-iodo-4-methylsulfanyl-pyrimidine as a white powder, m. p.=72-76° C.

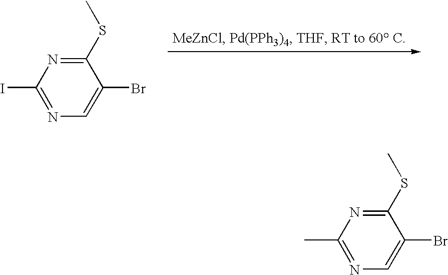

Into a parallel synthesis flask under argon charged with 50 mg of 5-bromo-2-iodo-4-methylsulfanyl-pyrimidine and 9 mg of tetrakistriphenylphosphinepalladium 1.51 ml of dry tetrahydrofuran were added. After stirring at rt for 10 min 76 [symbol] l of a solution of methylzinc chloride (2 M in tetrahydrofuran) were added and the mixture was heated at 60° C. for 5 h 30 min during which time a second portion of 20 [symbol] l of methylzinc chloride solution was added. After cooling to rt the mixture was poured into 10 ml of a saturated ammonium chloride solution, the aqueous phase extracted with ethyl acetate and the combined organic phases were washed with brine and dried over Na₂SO₄. Evaporation of the solvent gave a crude product which was purified by flash chromatography on silica gel yielding 15.1 mg of 5-bromo-2-methyl-4-methylsulfanyl-pyrimidine as a colourless oil.

LC-MS: Tᵣ=4.60 min (95.4%) (ES-MS: m/z 219.0 (M); 221 (M+2) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

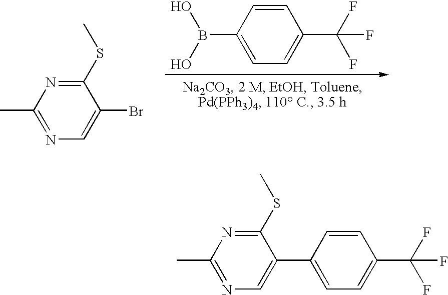

A solution of 1.94 g of sodium carbonate in 14.5 ml of water was added to a mixture of 1.57 g of 5-bromo-2-methyl-4-methylsulfanyl-pyrimidine, 1.43 g of 4-trifluorobenzeneboronic acid and 331 mg of tetrakistriphenylphospinpalladium in 14.5 ml of toluene and 14.5 ml of ethanol. After heating at 110° C. for 3.5 h the mixture was cooled to it and partitioned between 100 ml of ethyl acetate and 150 ml of water. The organic phase was extracted carefully with ethyl acetate, the combined organic layers were washed with brine, dried over Na₂SO₄ and evaporated to dryness. The crude product was purified by flash chromatography on silica gel with hexane/ethyl acetate 75:25 yielding 1.85 g of 2-methyl-4-methylsulfanyl-5-(4-trifluoromethyl-phenyl)-pyrimidine as a white solid, m.p.=109-112° C.

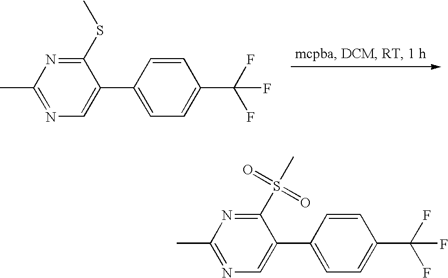

To a solution of 50 mg of 2-methyl-4-methylsulfanyl-5-(4-trifluoromethyl-phenyl)-pyrimidine in 3.5 ml of dichloromethane a solution of 87 mg of 3-chloroperbenzoic acid in 3.5 ml of dichloromethane was slowly added. After stirring for 1 h at it 15 ml of a solution of sodium bisulfite (5% in water) was added and the biphasic solution was transferred to a separatory funnel. The layers were shaken and separated. The aqueous phase was extracted two more times with dichloromethane, the combined organic phases were washed with a saturated solution of sodium carbonate and with brine, dried over Na₂SO₄ and evaporated to dryness. The residue was purified by flash chromatography on silica gel yielding 38.5 mg of 4-methanesulfonyl-2-methyl-5-(4-trifluoromethyl-phenyl)-pyrimidine as a white solid.

LC-MS: Tᵣ=4.69 min (100%) (ES-MS: m/z 317.0 (M+H) [Column: Nucleosil C-18HD, 4×70 mm, 3 μm, gradient CH₃CN/H₂O/TFA 0.05%: 20-100% CH₃CN (6 min.), 100% CH₃CN (1.5 min.), flow: 1 mL/min].

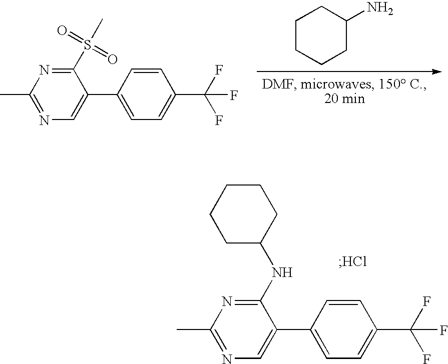

A mixture of 50 mg of 4-methanesulfonyl-2-methyl-5-(4-trifluoromethyl-phenyl)-pyrimidine, 2 ml of dimethyl formamide and 72 [symbol] l of cyclohexylamine was heated at 150° C. for 20 min in a microwave reactor (Biotage®). The crude mixture was evaporated to dryness and purified by flash chromatography on silica gel with hexane/ethyl acetate 1:1. The obtained product was dissolved in ethyl acetate and treated with a solution of 2 M HCl in ether to give 16 mg of cyclohexyl-[2-methyl-5-(4-trifluoromethyl-phenyl)-pyrimidin-4-yl]-amine as its hydrogen chloride salt: white solid, m.p.=220-225° C.

The following compounds were prepared in a similar way to Example 58, starting from 4-methanesulfonyl-2-methyl-5-(4-trifluoromethyl-phenyl)-pyrimidine and using the appropriate amine:

### EXAMPLE 59

### (1R,2R,4S)-Bicyclo[2.2.1]hept-2-yl-[2-methyl-5-(4-trifluoromethyl-phenyl)-pyrimidin-4-yl]amine

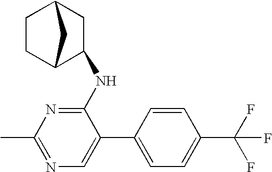

m.p.=92-96° C.

### EXAMPLE 60

### Adamantan-2-yl-[2-methyl-5-(4-trifluoromethyl-phenyl)-pyrimidin-4-yl]-amine

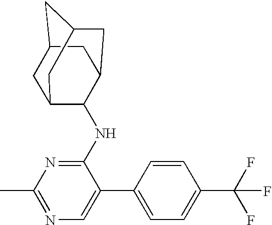

m.p.=149-154° C.

### EXAMPLE 61

### Cycloheptyl-[2-methyl-5-(4-trifluoromethyl-phenyl)-pyrimidin-4-yl]-amine

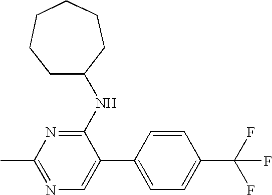

colourless oil

LC-MS: Tᵣ=4.65 min (100%) (ES-MS: m/z 350.2 (M+H) [Column: Nucleosil C-18HD, 4×70 mm, 3 μm, gradient CH₃CN/H₂O/TFA 0.1%: 20-100% CH₃CN (6 min.), 100% CH₃CN (1.5 min.), flow: 1 mL/min].

### EXAMPLE 62

### 3-[2-Methyl-5-(4-trifluoromethyl-phenyl)-pyrimidin-4-ylamino]-azepan-2-one

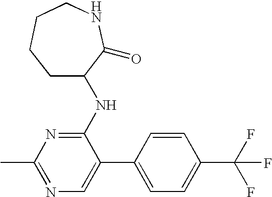

m.p.=191-196° C.

### EXAMPLE 63

### (1R,2S,4S)-Bicyclo[2.2.1]hept-2-yl-[2-methyl-5-(4-trifluoromethyl-phenyl)-pyrimidin-4-yl]-amine

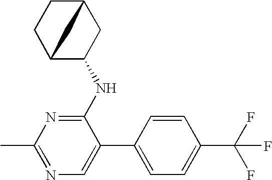

m.p.=131-135° C.

### EXAMPLE 64

### (6-Chloro-2-ethyl-5-phenyl-pyrimidin-4-yl)-cyclopentyl-amine

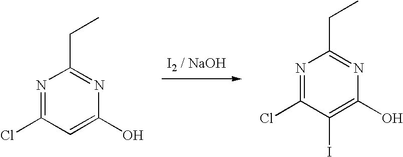

A mixture of 500 mg of 6-chloro-2-ethyl-pyrimidin-4-ol, 151 mg of sodium hydroxide and 3 ml of water was treated with 944 mg of iodine and heated to 50° C. After 4 h again 3 ml of water were added and stirring was continued for 1 h. After cooling to it the suspension was acidified with acetic acid and the precipitate filtered and crystallized from ethanol, yielding 547 mg of 6-chloro-2-ethyl-5-iodo-pyrimidin-4-ol as white needles, m.p.>230° C.

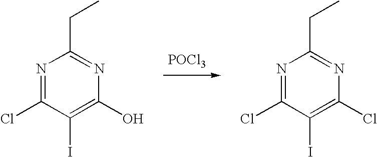

A mixture of 500 mg of 6-chloro-2-ethyl-5-iodo-pyrimidin-4-ol and 880 [symbol] l of phosphorus oxychloride was heated at reflux for 45 min. After cooling to it the mixture was poured on ice and basified with sodium carbonate to pH 9. The aqueous phase was extracted with dichloromethane, the combined organic layers were washed with saturated ammonium chloride solution and brine, dried over Na₂SO₄ and evaporated yielding 460 mg of 4,6-dichloro-2-ethyl-5-iodo-pyrimidine as a colourless oil.

¹H-NMR (CDCl₃, 360 MHz) δ: 2.85 (q, 2H); 1.25 (t, 3H)

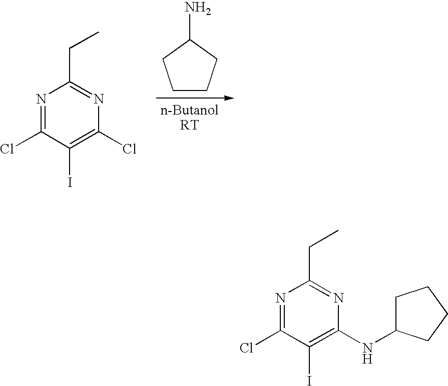

A mixture of 8.80 g of 4,6-dichloro-2-ethyl-5-iodo-pyrimidine, 5.55 ml of cyclopentylamine and 50 ml of n-butanol was stirred at rt for 24 h. The solvent was distilled off at the rotavap and the residue distributed between ethyl acetate and water. The aqueous phase was extracted with ethyl acetate, the combined organic layers were washed with brine, dried over Na₂SO₄ and evaporated yielding 9.20 g of (6-chloro-2-ethyl-5-iodo-pyrimidin-4-yl)-cyclopentyl-amine as a yellow oil.

MS (EI): m/z 351/353 (3:1) (M⁺)

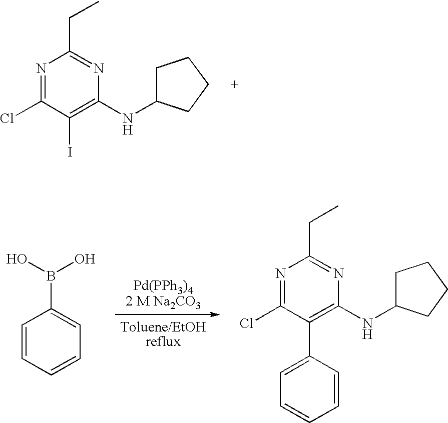

A mixture consisting of 2.5 g of (6-chloro-2-ethyl-5-iodo-pyrimidin-4-yl)-cyclopentyl-amine, 0.246 g of tetrakistriphenylphosphinpalladium, 0.954 g of phenylboronic acid, 8 ml of 2 M sodium carbonate solution, 4 ml of ethanol and 15 ml of toluene was heated at reflux for 24 h. After cooling to rt the aqueous phase was separated and extracted with ethyl acetate. The combined organic layers were washed with water and with brine, dried over Na₂SO₄ and evaporated. The residue was purified by flash chromatography on silica gel with cyclohexane/ethyl acetate 9:1 and the obtained product was treated with a solution of hydrogen chloride in ether yielding 2.41 g of (6-chloro-2-ethyl-5-phenyl-pyrimidin-4-yl)-cyclopentyl-amine hydrochloride salt, as a white powder, m.p.=112° C. (decomp.).

### EXAMPLE 65

### N,N′-Dicyclopentyl-2-ethyl-5-phenyl-pyrimidine-4,6-diamine

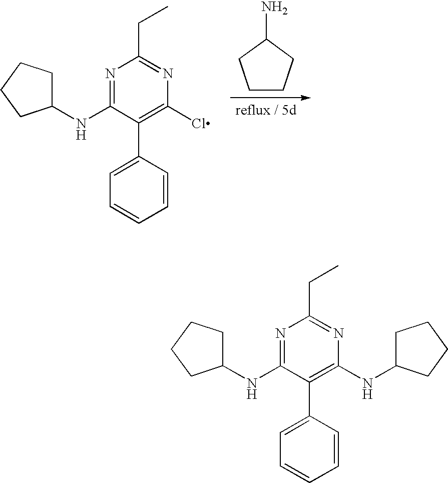

A mixture of 341 mg of (6-chloro-2-ethyl-5-phenyl-pyrimidin-4-yl)-cyclopentyl-amine and 3 ml of cyclopentylamine was heated at reflux for 8 days. After evaporation of the cyclopentylamine the residue was partitioned between water and diethyl ether. Ammonium hydroxide solution was added (pH=10) and the organic layer was separated, washed with water and brine, dried over Na₂SO₄ and evaporated to give a dark residue. Purification by flash chromatography on silica gel with cyclohexane/ethyl acetate 9:1 and crystallization of the obtained product from isopropanol/water yielded 162 mg of N,N′-dicyclopentyl-2-ethyl-5-phenyl-pyrimidine-4,6-diamine as a white powder, m.p. 70-71° C.

The biological assays on GABA_{B} receptors were performed following the procedure below:

GTPγ[³⁵S] binding. The assay mixtures contained 10 μg f membranes from a human GABA-B1b/rat GABA-B2 expressing CHO-K1 cell line in 50 mM Tris-HCl buffer, pH 7.7; 10 mM MgCl₂; 1.8 mM CaCl₂; 100 mM NaCl 30 μM guanosine 5′-diphosphate (30 μM; Sigma), 0.2 nM [³⁵S]GTP(γ)S, and test compounds (Urwyler et al, 2001). 96-well Packard Pico-plates (300 μl volume) were used. Non-specific binding was measured in the presence of unlabelled GTP(γ)S (10 μM, Sigma). The reagents were incubated for 40 min at room temperature and subsequently filtered (Packard unifilter-GF/C). After two washes with assay buffer as above the plates were dried for one hour at 50° C., 50 μl of scintillation solution (Microscint) was added and the radioactivity counted. For data analysis, non-specific binding was subtracted from all the other values; the compound effects were expressed relative to basal activity. Prism 3.0 software (Graph Pad software, San Diego, Calif.) was used for all data calculations. (Urwyler S, Mosbacher J, Lingenhoehl K, Heid J, Hofstetter K, Froestl W, Bettler B, Kaupmann K. Mol. Pharmacol. 2001, 60:963-71).

Compounds of the invention typically have a biological activity (BA) summarized in the table below:

| | 20 μM of GABA | | 1 μM of GABA | |
|---|---|---|---|---|
| | 2.5 μM of Cpd | 25 μM of Cpd | 2.5 μM of Cpd | 25 μM of Cpd |
| Biological | 90 < BA < 165 or | 130 < BA < 220 or | 40 < BA < 80 or | 65 < BA < 140 or |
| activity (%) | more | more | more | more |
| Biological | 159 | 192 | 77 | 129 |
| activity (%) of | | | | |
| Example 62 | | | | |

We claim:

1. A compound of the formula 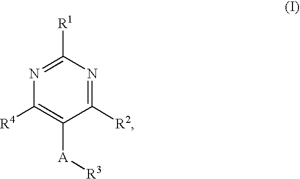
in free base form or in acid addition salt form, wherein
R¹ represents alkyl, halogenalkyl, alkoxy, halogenalkoxy, alkylthio, halogenalkylthio, alkylamino or halogenalkylamino;
R² represents halogen, hydroxy or substituted amino, the substituent(s) being selected from the group consisting of hydrogen, unsubstituted or substituted alkyl, unsubstituted or substituted cycloalkyl, unsubstituted or substituted bicycloalkyl, unsubstituted or substituted adamantyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl;
R³ represents halogen, halogenalkyl, nitro, unsubstituted or substituted aryl or unsubstituted or substituted heteroaryl;
R⁴ represents hydrogen, halogen, hydroxy, alkynyl, trialkylsilylalkynyl or substituted amino, the substituent(s) being selected from the group consisting of hydrogen, unsubstituted or substituted alkyl, unsubstituted or substituted cycloalkyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl; and
A represents a bond, alkandiyl, alkendiyl or alkyndiyl; and
wherein additionally the amino nitrogen atom of a substituted amino group R² can be connected via a direct bond or via a carbonyl group with a ring carbon atom of an unsubstituted or substituted aryl or an unsubstituted or substituted heteroaryl group R³.

2. The compound of formula (I-A) according to claim 1

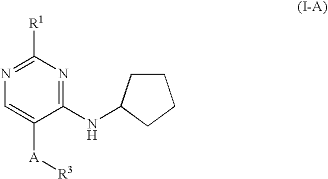

3. The compound of formula (I-B) according to claim 1 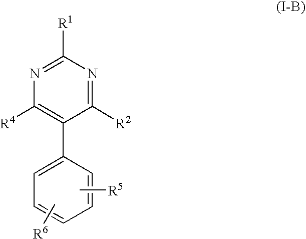
wherein
R⁵ and R⁶ independently represent fluoro, chloro, bromo, jodo, cyano, nitro, amino, PO₃H₂, H₂NC(O), methyl, ethyl, n- or iso-propyl, n-, iso-, sec- or tert-butyl, fluormethyl, difluormethyl trifluormethyl, chlormethyl, dichlormethyl, methoxy, ethoxy, n- or iso-propoxy, n-, iso-, sec- or tert-butoxy, fluormethoxy, difluormethoxy, trifluormethoxy, chlormethoxy, dichiormethoxy, methoxycarbonyl, ethoxycarbonyl, trifluormethoxycarbonyl, C₁₋₄ methylthio, methylsulfinyl, methylsulfonyl, trifluormethylthio.

4. A process for the preparation of a compound of formula (I) 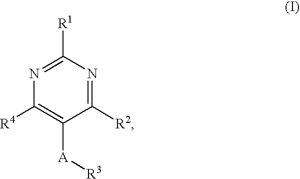
in free base form or in acid addition salt form, wherein
R¹ represents alkyl, halogenalkyl, alkoxy, halogenalkoxy, alkylthio, halogenalkylthio, alkylamino or halogenalkylamino;
R² represents halogen, hydroxy or substituted amino, the substituent(s) being selected from the group consisting of hydrogen, unsubstituted or substituted alkyl, unsubstituted or substituted cycloalkyl unsubstituted or substituted bicycloalkyl, unsubstituted or substituted adamantyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl;
R³ represents halogen, halogenalkyl, nitro, unsubstituted or substituted aryl or unsubstituted or substituted heteroaryl;
R⁴ represents hydrogen, halogen, hydroxy, alkynyl, trialkylsilylalkynyl or substituted amino, the substituent(s) being selected from the group consisting of hydrogen, unsubstituted or substituted alkyl, unsubstituted or substituted cycloalkyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl; and
A represents a bond, alkandiyl, alkendiyl or alkyndiyl;
wherein additionally the amino nitrogen atom of a substituted amino group R² can be connected via a direct bond or via a carbonyl group with a ring carbon atom of an unsubstituted or substituted aryl or an unsubstituted or substituted heteroaryl group R³, comprising:
a: —in case A represents a single bond—the step of reacting a compound of formula (II) 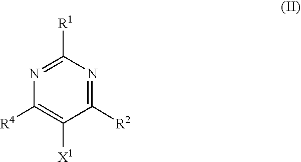
wherein R¹, R² and R⁴ are as defined above, and X¹ represents Br or I, with a compound of formula (III) 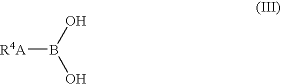
wherein R³ is as defined above and A represents a single bond, in a Suzuki type coupling reaction and recovering the resulting compound of formula (I) in free, base or acid addition salt form; or
b: —in case A represents alkandiyl, alkendiyl or alkyndiyl—the step of reacting a compound of formula (II) 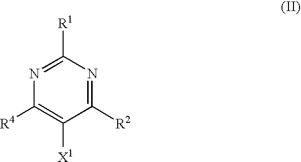
wherein R¹, R² and R⁴ are as defined above, and XI represents Br or I with a compound of formula (IV) 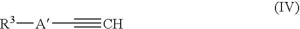
wherein R³ is as defined above and A represents a single bond (in case A represents C₂) or an alkandiyl which is two C atoms shorter than A in the compound of formula (I), in a Sonogashira type coupling reaction, and recovering the resulting compound of formula (I) in free base or acid addition salt form, and which in each case may optionally be followed by reduction, oxidation or functionalisation of the resulting compound and/or by cleavage of protecting groups optionally present, and of recovering the so obtainable compound of the formula I in free base form or in acid addition salt form.

5. (canceled)

6. (canceled)

7. A pharmaceutical composition, comprising:
a compound of claim 1 in free base or pharmaceutically acceptable acid addition salt form, in association with a pharmaceutical carrier or diluent.

8. (canceled)

9. (canceled)

10. (canceled)

11. A method of treating disorders associated with irregularities of the glutamatergic signal transmission, and nervous system disorders mediated full or in part by GABA B, comprising:
administering to a subject in need of such treatment a therapeutically effective amount of the compound of claim 1 in free base or pharmaceutically acceptable acid addition salt form.

12. A compound of formula (II-A) 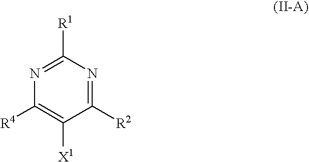
wherein
R¹ represents alkyl, halogenalkyl, alkoxy, halogenalkoxy, alkylthio, halogenalkylthio, alkylamino or halogenalkylamino;
R⁴ represents hydrogen, halogen, hydroxy, alkynyl, trialkylsilylalkynyl or substituted amino, the substituent(s) being selected from the group consisting of hydrogen, unsubstituted or substituted alkyl, unsubstituted or substituted cycloalkyl, unsubstituted or substituted alkyl(CO), unsubstituted or substituted cycloalkyl(CO), unsubstituted or substituted aryl, unsubstituted or substituted heteroaryl, unsubstituted or substituted heterocyclyl, unsubstituted or substituted aralkyl, unsubstituted or substituted heteroarylalkyl and unsubstituted or substituted heterocyclylalkyl;
R² represents halogen, hydroxy or substituted amino, the substitutents being selected from the group consisting of hydrogen, alkyl, cycloalkyl;
X¹ represents I or Br.

13. The method of claim 11, wherein the disorder is schizophrenia or depression.
